(19) United States
(12) Patent Application Publication
(10) Pub. No.: US 2023/0286999 A1
(43) Pub. Date: Sep. 14, 2023

(54) ORGANIC COMPOUND, LIGHT-RECEIVING DEVICE, LIGHT-EMITTING AND LIGHT-RECEIVING APPARATUS, AND ELECTRONIC DEVICE

(71) Applicant: Semiconductor Energy Laboratory Co., Ltd., Atsugi-shi (JP)

(72) Inventors: Anna TADA, Isehara (JP); Sachiko KAWAKAMI, Atsugi (JP); Daisuke KUBOTA, Atsugi (JP); Taisuke KAMADA, Niiza (JP); Kazuki KAJIYAMA, Atsugi (JP)

(21) Appl. No.: 18/113,672

(22) Filed: Feb. 24, 2023

(30) Foreign Application Priority Data
Mar. 11, 2022 (JP) 2022-037934

(51) Int. Cl. C07D 495/04; C07D 519/00; H10K 85/60; H10K 50/16; H10K 50/11

(52) U.S. Cl. CPC C07D 495/04; C07D 519/00; H10K 85/6576; H10K 50/16; H10K 50/11; H10K 2102/301

(57) ABSTRACT

A novel organic compound that is highly convenient, useful, or reliable is provided. An organic compound represented by General Formula (G1) is provided. In General Formula (G1), D¹ represents a thiophene-diyl group, a furan-diyl group, a thiophene-containing heteroarylene group, or a furan-containing heteroarylene group; Ar¹ and Ar² each independently represent a heteroarylene group or an arylene group; A¹ and A² each independently represent hydrogen, deuterium, a nitro group, an alkyl group, a halogen, an alkyl halide group, a cyano group, an alkoxy group, a vinyl group, or a formyl group; n₁ represents an integer of 1 or more; and m₁ and k₁ each independently represent an integer of 0 to 3.

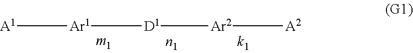

### BACKGROUND OF THE INVENTION

### 1. Field of the Invention

One embodiment of the present invention relates to an organic compound, a light-receiving device, a light-emitting and light-receiving apparatus, and an electronic device.

Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specific examples of the technical field of one embodiment of the present invention disclosed in this specification include a semiconductor device, a display device, a light-emitting apparatus, a power storage device, a memory device, a method for driving any of them, and a method for manufacturing any of them.

### 2. Description of the Related Art

Light-receiving devices using organic compounds have increasingly been put into practical use. In the basic structure of such light-receiving devices, an organic compound layer containing a photoelectric conversion material (an active layer) is located between a pair of electrodes. This device absorbs light energy to generate carriers, whereby electrons from the photoelectric conversion material can be obtained.

For example, a functional panel in which a pixel provided in a display region includes a light-emitting element (light-emitting device) and a photoelectric conversion element (light-receiving device) is known (Patent Document 1). For example, the functional panel includes a first driver circuit, a second driver circuit, and a region. The first driver circuit supplies a first selection signal, the second driver circuit supplies a second selection signal and a third selection signal, and the region includes a pixel. The pixel includes a first pixel circuit, a light-emitting element, a second pixel circuit, and a photoelectric conversion element. The first pixel circuit is supplied with the first selection signal, the first pixel circuit obtains an image signal on the basis of the first selection signal, the light-emitting element is electrically connected to the first pixel circuit, and the light-emitting element emits light on the basis of the image signal. The second pixel circuit is supplied with the second selection signal and the third selection signal in a period during which the first selection signal is not supplied, the second pixel circuit obtains an imaging signal on the basis of the second selection signal and supplies the imaging signal on the basis of the third selection signal, and the photoelectric conversion element is electrically connected to the second pixel circuit and generates the imaging signal.

### REFERENCE

[Patent Document 1] PCT International Publication No. WO2020/152556

### SUMMARY OF THE INVENTION

An object of one embodiment of the present invention is to provide a novel light-receiving device that is highly convenient, useful, or reliable. Another object is to provide a novel light-emitting and light-receiving apparatus that is highly convenient, useful, or reliable. Another object is to provide a novel electronic device that is highly convenient, useful, or reliable. Another object is to provide a novel light-receiving device, a novel light-emitting and light-receiving apparatus, or a novel electronic device.

Note that the description of these objects does not preclude the existence of other objects. In one embodiment of the present invention, there is no need to achieve all these objects. Other objects will be apparent from and can be derived from the description of the specification, the drawings, the claims, and the like.

One embodiment of the present invention is an organic compound represented by General Formula (G1).

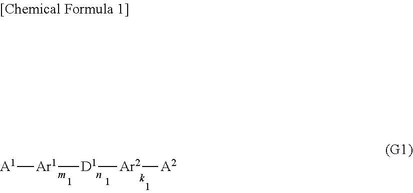

In General Formula (G1), D¹ represents a substituted or unsubstituted thiophene-diyl group, a substituted or unsubstituted furan-diyl group, a substituted or unsubstituted thiophene-containing heteroarylene group having 4 to 30 carbon atoms, or a substituted or unsubstituted furan-containing heteroarylene group having 4 to 30 carbon atoms. Ar¹ and Ar² each independently represent a heteroarylene group having 2 to 30 carbon atoms and having 1 or more substituents or an arylene group having 6 to 30 carbon atoms and having 1 or more substituents. A¹ and A² each independently represent hydrogen, deuterium, a nitro group, a substituted or unsubstituted alkyl group having 1 to 6 carbon atoms, a halogen, a substituted or unsubstituted alkyl halide group having 1 to 6 carbon atoms, a cyano group, a substituted or unsubstituted alkoxy group having 1 to 6 carbon atoms, a vinyl group having 1 to 3 substituents, or a formyl group. Furthermore, n₁ represents an integer of 1 or more, and m₁ and k₁ each independently represent an integer of 0 to 3. At least one of the substituents that D¹, Ar¹, and Ar² have has a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms. In the case where m₁ and k₁ are 0, at least one of the substituents that D¹ has a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms.

In General Formula (G1) above, D¹ is represented by any one of General Formulas (g1-1-1) to (g1-1-5).

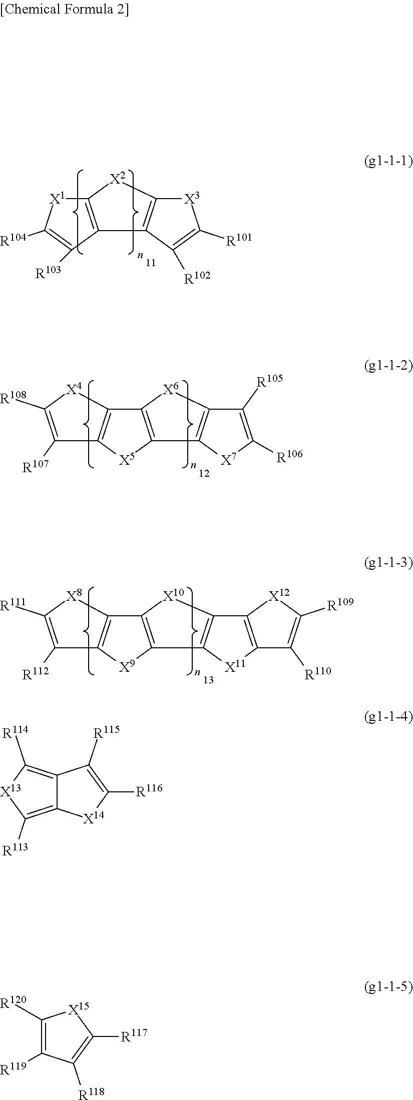

In General Formulas (g1-1-1) to (g1-1-5), n₁₁ represents an integer of 0 to 10, and n₁₂ and n₁₃ each independently represent an integer of 0 to 4. X¹ to X¹⁵ each independently represent oxygen or sulfur. One of R¹⁰ and R¹⁰², one of R¹⁰⁵ and R¹⁰⁶, or one of R¹⁰⁹ and R¹¹⁰ is bonded to one of Ar¹ or A¹ and Ar² or A². One of R¹⁰³ and R¹⁰⁴, one of R¹⁰⁷ and R¹⁰⁸, or one of R¹¹¹ and R¹¹² is bonded to other of Ar¹ or A¹ and Ar² or A². Any one of R¹¹³ to R¹¹⁶ is bonded to Ar¹ or A¹. Another one of R¹¹³ to R¹¹⁶ is bonded to Ar² or A². Any one of R¹¹⁷ to R¹²⁰ is bonded to Ar¹ or A¹. Another one of R¹¹⁷ to R¹²⁰ is bonded to Ar² or A². Groups bonded to none of Ar¹, A¹, Ar², and A² among R¹⁰¹ to R¹²⁰ each independently represent hydrogen, deuterium, a branched alkyl group having 3 to 20 carbon atoms, a straight-chain alkyl group having 7 or more carbon atoms, a cycloalkyl group having 3 to 10 carbon atoms, a branched alkoxy group having 3 to 20 carbon atoms, a straight-chain alkoxy group having 7 or more carbon atoms, a substituted or unsubstituted aryl group having 6 to 30 carbon atoms, a substituted or unsubstituted heteroaryl group having 2 to 30 carbon atoms, a branched alkyl halide group having 3 to 20 carbon atoms, a straight-chain alkyl halide group having 7 or more carbon atoms, or a halogen.

In General Formula (G1) above, each of A¹ and A² is represented by General Formula (g1-2).

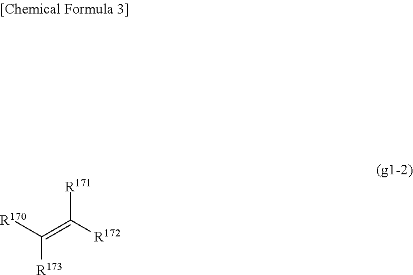

In General Formula (g1-2), R¹⁷⁰ to R¹⁷² each independently represent hydrogen, deuterium, a cyano group, fluorine, chlorine, a nitro group, a substituted or unsubstituted alkyl halide group having 1 to 6 carbon atoms, or a substituted or unsubstituted alkoxy group having 1 to 6 carbon atoms, and R¹⁷³ is bonded to one of Ar¹ and Ar² or D¹.

In General Formula (G1) above, Ar¹ and Ar² each independently represent a substituted or unsubstituted thiophene-diyl group, a substituted or unsubstituted furan-diyl group, a substituted or unsubstituted phenylene group, or a substituted or unsubstituted naphthalene-diyl group.

One embodiment of the present invention is an organic compound represented by any one of General Formulas (G1-1) to (G1-3).

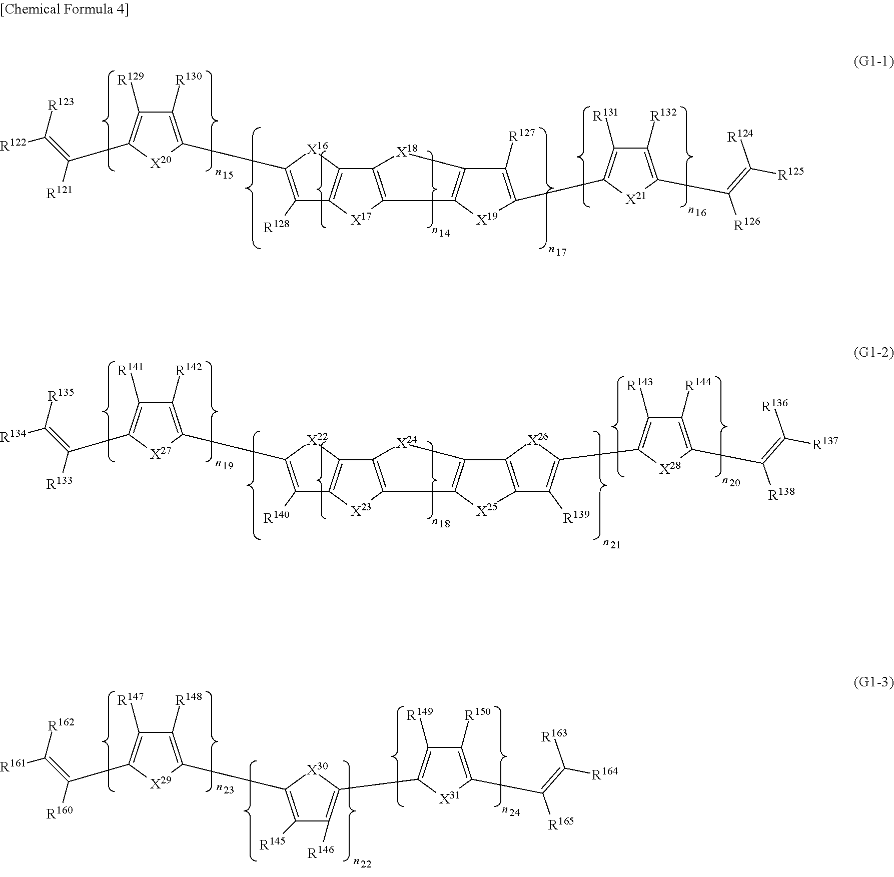

In General Formulas (G1-1) to (G1-3), X¹⁶ to X³¹ each independently represent oxygen or sulfur; n₁₄, n₁₈, and n₂₂ each independently represent an integer of 0 to 4; n₁₅, n₁₆, n₁₉, n₂₀, n₂₃, and n₂₄ each independently represent an integer of 0 to 3; and n₁₇, n₂₁, and n₂₂ represent an integer of 1 to 3. R¹²⁷ to R¹³², R¹³⁹ to R¹⁴⁴, and R¹⁴⁵ to R¹⁵⁰ each independently represent hydrogen, deuterium, a branched alkyl group having 3 to 20 carbon atoms, a straight-chain alkyl group having 7 or more carbon atoms, a cycloalkyl group having 3 to 10 carbon atoms, a branched alkoxy group having 3 to 20 carbon atoms, a straight-chain alkoxy group having 7 or more carbon atoms, a substituted or unsubstituted aryl group having 6 to 30 carbon atoms, a substituted or unsubstituted heteroaryl group having 2 to 30 carbon atoms, a branched alkyl halide group having 3 to 20 carbon atoms, a straight-chain alkyl halide group having 7 or more carbon atoms, or a halogen. At least one of R¹²⁷ to R¹³², at least one of R¹³⁹ to R¹⁴⁴, and at least one of R¹⁴⁵ to R¹⁵⁰ each independently represent a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms. R¹²¹ to R¹²⁶, R¹³³ to R¹³⁸, and R¹⁶⁰ to R¹⁶⁵ each independently represent hydrogen, deuterium, a cyano group, fluorine, chlorine, a substituted or unsubstituted alkyl halide group having 1 to 6 carbon atoms, or a substituted or unsubstituted alkoxy group having 1 to 6 carbon atoms. At least one of R¹²¹ to R¹²⁶ at least one of R¹³³ to R¹³⁸, and at least one of R¹⁶⁰ to R¹⁶⁵ represent a cyano group, fluorine, chlorine, a nitro group, a substituted or unsubstituted alkyl halide group having 1 to 6 carbon atoms, or a substituted or unsubstituted alkoxy group having 1 to 6 carbon atoms.

One embodiment of the present invention is an organic compound represented by Structural Formula (100) or Structural Formula (200).

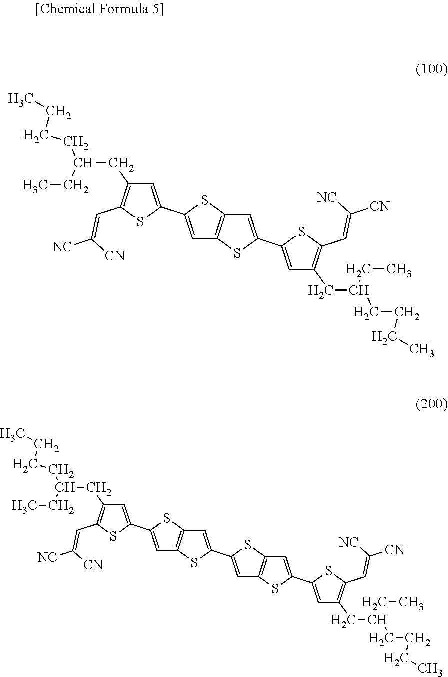

One embodiment of the present invention is a light-receiving device including a first electrode, a second electrode, and an organic compound layer. The organic compound layer is positioned between the first electrode and the second electrode, the organic compound layer contains an organic compound, and the organic compound is represented by General Formula (G1).

In the above-described light-receiving device, the organic compound represented by General Formula (G1) above can be used for an active layer.

In the above-described light-receiving device, the organic compound represented by General Formula (G1) above can be used for an electron-transport layer.

The above-described light-receiving device includes a light-emitting layer.

One embodiment of the present invention is a light-emitting and light-receiving apparatus including the above-described light-receiving device and a light-emitting device.

One embodiment of the present invention is an electronic device including the above-described light-emitting and light-receiving apparatus; and a sensor unit, an input unit, or a communication unit.

One embodiment of the present invention is an electronic device including the above-described light-emitting and light-receiving apparatus and at least one of a microphone, a camera, an operation button, a connection terminal, and a speaker.

Although the block diagram in drawings attached to this specification shows components classified based on their functions in independent blocks, it is difficult to classify actual components based on their functions completely, and one component can have a plurality of functions.

With one embodiment of the present invention, a novel organic compound that is highly convenient, useful, or reliable can be provided. With one embodiment of the present invention, a novel light-receiving device that is highly convenient, useful, or reliable can be provided. A novel light-emitting and light-receiving apparatus that is highly convenient, useful, or reliable can be provided. A novel electronic device that is highly convenient, useful, or reliable can be provided. A novel light-receiving device, a novel light-emitting and light-receiving apparatus, or a novel electronic device can be provided.

Note that the description of these effects does not preclude the existence of other effects. One embodiment of the present invention does not necessarily have all these effects. Other effects will be apparent from and can be derived from the description of the specification, the drawings, the claims, and the like.

### BRIEF DESCRIPTION OF THE DRAWINGS

In the accompanying drawings:

FIGS. 1A to 1C illustrate a light-receiving device of one embodiment of the present invention;

FIGS. 2A to 2C illustrate a light-emitting and light-receiving apparatus of one embodiment of the present invention;

FIGS. 3A and 3B each illustrate a light-emitting and light-receiving apparatus of one embodiment of the present invention;

FIGS. 4A to 4E each illustrate a structure of a light-emitting device of an embodiment;

FIGS. 5A to 5D each illustrate a light-emitting and light-receiving apparatus of an embodiment;

FIGS. 6A to 6C illustrate a method for manufacturing a light-emitting and light-receiving apparatus of an embodiment;

FIGS. 7A to 7C illustrate the method for manufacturing the light-emitting and light-receiving apparatus of the embodiment;

FIGS. 8A to 8C illustrate the method for manufacturing the light-emitting and light-receiving apparatus of the embodiment;

FIGS. 9A to 9D illustrate the method for manufacturing the light-emitting and light-receiving apparatus of the embodiment;

FIGS. 10A to 10E illustrate the method for manufacturing the light-emitting and light-receiving apparatus of the embodiment;

FIGS. 11A to 11F illustrate a light-emitting and light-receiving apparatus of an embodiment and pixel arrangements;

FIGS. 12A to 12C illustrate pixel circuits of an embodiment;

FIG. 13 illustrates a light-emitting and light-receiving apparatus of an embodiment;

FIGS. 14A to 14E illustrate electronic devices of an embodiment;

FIGS. 15A to 15E illustrate electronic devices of an embodiment;

FIGS. 16A and 16B illustrate electronic devices of an embodiment;

FIG. 17 shows a ¹H NMR spectrum of an organic compound formed in Example 1; and

FIG. 18 shows a ¹H NMR spectrum of an organic compound formed in Example 2.

### DETAILED DESCRIPTION OF THE INVENTION

Embodiments will be described in detail with reference to the drawings. Note that the embodiments of the present invention are not limited to the following description, and it will be readily appreciated by those skilled in the art that modes and details of the present invention can be modified in various ways without departing from the spirit and scope of the present invention. Therefore, the present invention should not be construed as being limited to the description in the following embodiments. Note that in structures of the invention described below, the same portions or portions having similar functions are denoted by the same reference numerals in different drawings, and the description thereof is not repeated.

### Embodiment 1

In this embodiment, an organic compound of one embodiment of the present invention is described.

### Example 1 of Organic Compound

The organic compound described in this embodiment is an organic compound represented by General Formula (G1) below.

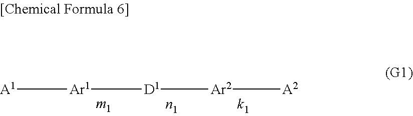

In General Formula (G1) above, D¹ represents a substituted or unsubstituted thiophene-diyl group, a substituted or unsubstituted furan-diyl group, a substituted or unsubstituted thiophene-containing heteroarylene group having 4 to 30 carbon atoms, or a substituted or unsubstituted furan-containing heteroarylene group having 4 to 30 carbon atoms. In addition, Ar¹ and Ar² each independently represent a heteroarylene group having 2 to 30 carbon atoms and having 1 or more substituents or an arylene group having 6 to 30 carbon atoms and having 1 or more substituents. Furthermore, A¹ and A² each independently represent hydrogen, deuterium, a nitro group, a substituted or unsubstituted alkyl group having 1 to 6 carbon atoms, a halogen, a substituted or unsubstituted alkyl halide group having 1 to 6 carbon atoms, a cyano group, a substituted or unsubstituted alkoxy group having 1 to 6 carbon atoms, a vinyl group having 1 to 3 substituents, or a formyl group.

Moreover, n₁ represents an integer of 1 or more, and m₁ and k₁ each independently represent an integer of 0 to 3. Furthermore, at least one of the substituents that D¹, Ar¹, and Ar² have has a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms.

Furthermore, in the case where m₁ and k₁ are 0, at least one of the substituents that D¹ has a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms.

Here, when the volume of the substituent bonded to D¹, Ar¹, or Ar² in General Formula (G1) above is increased, the stacking interaction due to the dispersion force between aromatic rings can be suppressed. The inhibition of aggregation of molecules or crystallization caused by the stacking interaction can improve the solubility of the organic compound in a solvent. In contrast, when the molecular weight of the organic compound itself becomes too high, the solubility tends to be decreased.

Therefore, when at least one of the substituents that D¹, Ar¹, and Ar² have has a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms, the stacking interaction between molecules can be suppressed, and the solubility in a solvent can be improved.

In General Formula (G1) above, D¹ is preferably represented by any one of General Formulas (g1-1-1) to (g1-1-5).

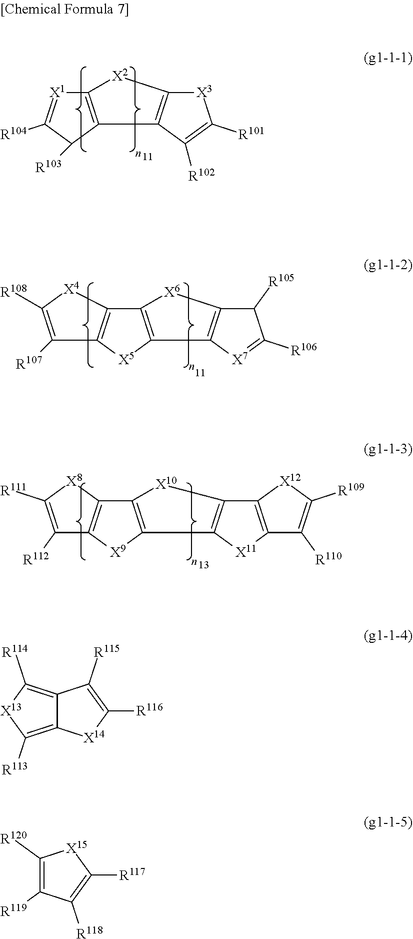

In General Formulas (g1-1-1) to (g1-1-5), n₁₁ represents an integer of 0 to 10, n₁₂ and n₁₃ each independently represent an integer of 0 to 4, X¹ to X¹⁵ each independently represent oxygen or sulfur, one of R¹⁰¹ and R¹⁰², one of R¹⁰⁵ and R¹⁰⁶, or one of R¹⁰⁹ and R¹¹⁰ is bonded to one of Ar¹ or A¹ and Ar² or A², one of R¹⁰³ and R¹⁰⁴, one of R¹⁰⁷ and R⁰, or one of R¹¹¹ and R¹¹² is bonded to the other of Ar¹ or A¹ and Ar² or A², any one of R¹¹³ to R¹¹⁶ is bonded to Ar¹ or A¹, another one of R¹¹³ to R¹¹⁶ is bonded to Ar² or A², any one of R¹¹⁷ to R¹²⁰ is bonded to Ar¹ or A¹, and another one of R¹¹⁷ to R¹²⁰ is bonded to Ar² or A². Groups bonded to none of Ar¹, A¹, Ar², and A² among R¹⁰¹ to R¹²⁰ each independently represent hydrogen, deuterium, a branched alkyl group having 3 to 20 carbon atoms, a straight-chain alkyl group having 7 or more carbon atoms, a cycloalkyl group having 3 to 10 carbon atoms, a branched alkoxy group having 3 to 20 carbon atoms, a straight-chain alkoxy group having 7 or more carbon atoms, a substituted or unsubstituted aryl group having 6 to 30 carbon atoms, a substituted or unsubstituted heteroaryl group having 2 to 30 carbon atoms, a branched alkyl halide group having 3 to 20 carbon atoms, a straight-chain alkyl halide group having 7 or more carbon atoms, or a halogen.

Note that the substituents represented by General Formulas (g1-1-1) to (g1-1-5) above are merely examples and D¹ that can be used in General Formula (G1) above is not limited thereto.

In General Formula (G1) above, Ar¹ and Ar² each independently represent a substituted or unsubstituted thiophene-diyl group, a substituted or unsubstituted furan-diyl group, a substituted or unsubstituted phenylene group, or a substituted or unsubstituted naphthalene-diyl group.

That is, in the case where m₁ and k₁ are both 0 in General Formula (G1) above, at least one of the substituents that D¹ has a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms.

Moreover, in General Formula (G1) above, each of A¹ and A² is preferably represented by General Formula (g1-2).

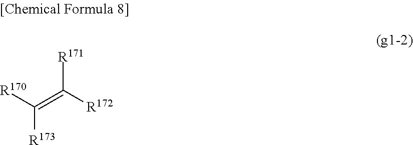

In General Formula (g1-2), R¹⁷⁰ to R¹⁷² each independently represent hydrogen, deuterium, a cyano group, fluorine, chlorine, a nitro group, a substituted or unsubstituted alkyl halide group having 1 to 6 carbon atoms, or a substituted or unsubstituted alkoxy group having 1 to 6 carbon atoms, and R¹⁷³ is bonded to one of Ar¹ and Ar² or D¹.

Note that the substituent represented by General Formula (g1-2) above is merely an example and A¹ and A² that can be used in General Formula (G1) above are not limited thereto.

The organic compound of one embodiment of the present invention can absorb light in a wide wavelength range including the visible light region. Thus, for example, the organic compound can be suitably used for an active layer of a light-receiving device. For example, the organic compound can be suitably used for a layer in contact with an active layer of a light-receiving device.

The organic compound of one embodiment of the present invention can be highly purified owing to its high solubility, and accordingly a highly reliable light-receiving device can be provided. The organic compound has a relatively low sublimation temperature and thus can be easily purified by sublimation. In particular, in the case where the organic compound is used in a device, the organic compound can be deposited at a low temperature in a manufacturing step with heating, such as a vacuum evaporation step, for example. Thus, deterioration of other materials used in the device, particularly deterioration of an organic compound, can be suppressed. As a result, manufacturing costs can be reduced without impairing the characteristics of the novel organic compound that is highly convenient, useful, or reliable.

With the organic compound, a device that can receive light in a wide wavelength range including the visible light region can be provided. A light-receiving device capable of operation at low voltage can be provided. Furthermore, a high-efficiency photoelectric conversion device can be provided. Moreover, the organic compound of one embodiment of the present invention can be synthesized by a variety of methods, so that the molecular design can be flexible.

### Example 2 of Organic Compound

The organic compounds described in this embodiment are organic compounds represented by General Formulas (G1-1) to (G1-3) below.

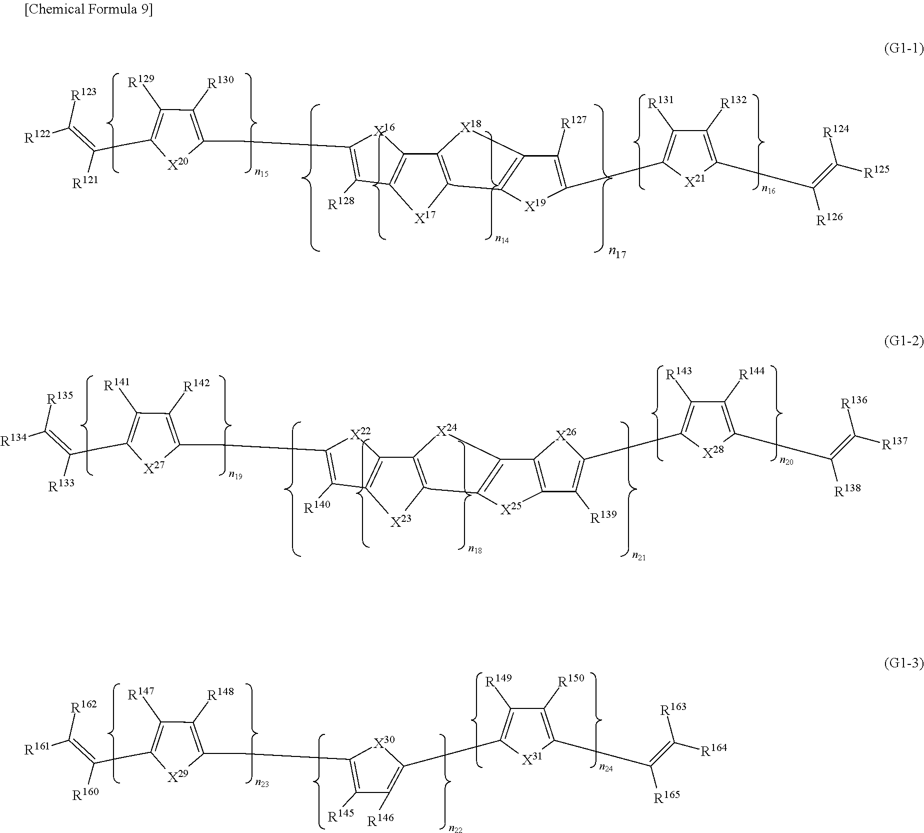

In General Formulas (G1-1) to (G1-3), X¹⁶ to X³¹ each independently represent oxygen or sulfur; n₁₄, n₁₈, and n₂₂ each independently represent an integer of 0 to 4; n₁₈, n₁₆, n₁₉, n₂₀, n₂₃, and n₂₄ each independently represent an integer of 0 to 3; and n₁₇, n₂₁, and n₂₂ represent an integer of 1 to 3. In addition, R¹²⁷ to R¹³², R¹³⁹ to R¹⁴⁴, and R¹⁴⁵ to R¹⁵⁰ each independently represent hydrogen, deuterium, a branched alkyl group having 3 to 20 carbon atoms, a straight-chain alkyl group having 7 or more carbon atoms, a cycloalkyl group having 3 to 10 carbon atoms, a branched alkoxy group having 3 to 20 carbon atoms, a straight-chain alkoxy group having 7 or more carbon atoms, a substituted or unsubstituted aryl group having 6 to 30 carbon atoms, a substituted or unsubstituted heteroaryl group having 2 to 30 carbon atoms, a branched alkyl halide group having 3 to 20 carbon atoms, a straight-chain alkyl halide group having 7 or more carbon atoms, or a halogen. Furthermore, at least one of R¹²⁷ to R¹³², at least one of R¹³⁹ to R¹⁴⁴, and at least one of R¹⁴⁵ to R¹⁵⁰ each independently represent a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms. Moreover, R¹²¹ to R¹²⁶, R¹³³ to R¹³⁸, and R¹⁶⁰ to R¹⁶⁵ each independently represent hydrogen, deuterium, a cyano group, fluorine, chlorine, a substituted or unsubstituted alkyl halide group having 1 to 6 carbon atoms, or a substituted or unsubstituted alkoxy group having 1 to 6 carbon atoms. Furthermore, at least one of R¹²¹ to R¹²⁶, at least one of R¹³³ to R¹³⁸, and at least one of R¹⁶⁰ to R¹⁶⁵ represent a cyano group, fluorine, chlorine, a nitro group, a substituted or unsubstituted alkyl halide group having 1 to 6 carbon atoms, or a substituted or unsubstituted alkoxy group having 1 to 6 carbon atoms.

The organic compound of one embodiment of the present invention can absorb light in a wide wavelength range including the visible light region. Thus, for example, the organic compound can be suitably used for an active layer of a light-receiving device. For example, the organic compound can be suitably used for a layer in contact with an active layer of a light-receiving device.

The organic compound of one embodiment of the present invention can be highly purified owing to its high solubility, and accordingly a highly reliable light-receiving device can be provided. The organic compound has a relatively low sublimation temperature and thus can be easily purified by sublimation. In particular, in the case where the organic compound is used in a device, the compound can be deposited at a low temperature in a manufacturing step with heating, such as a vacuum evaporation step, for example. Thus, deterioration of other materials used in the device, particularly deterioration of an organic compound, can be suppressed. As a result, manufacturing costs can be reduced without impairing the characteristics of the novel organic compound that is highly convenient, useful, or reliable.

### Specific Examples of Organic Compound

Specific structural formulas of the above-described organic compound of one embodiment of the present invention are shown below.

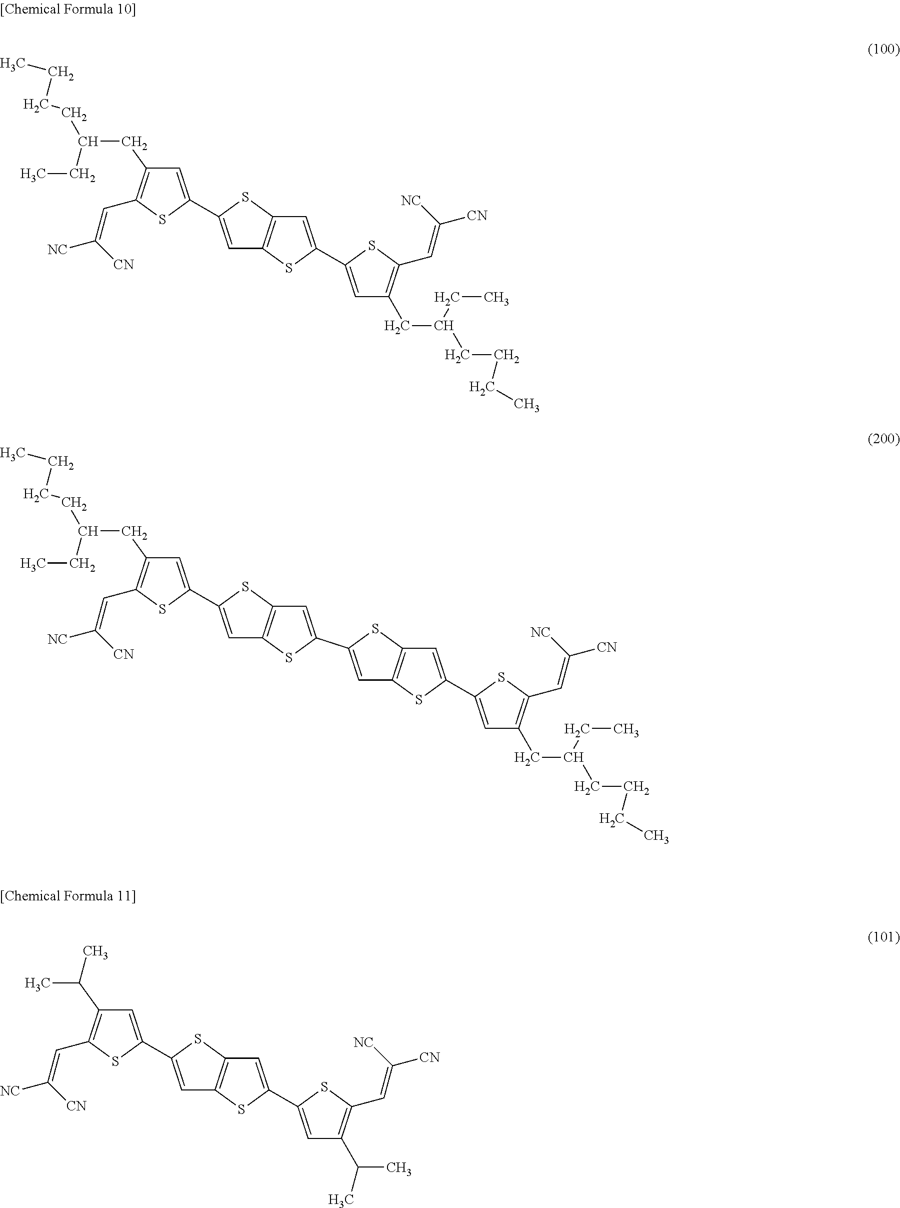

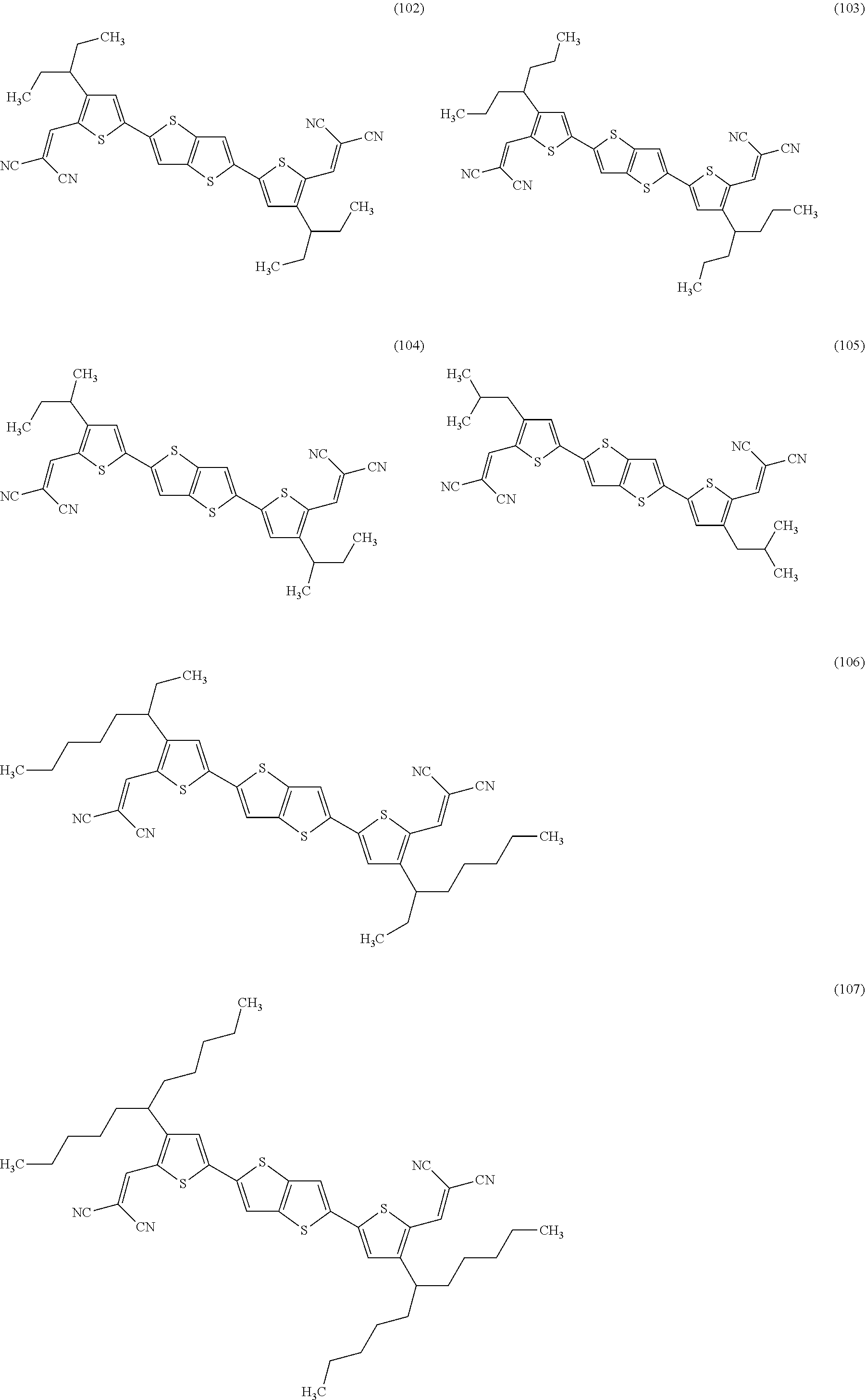

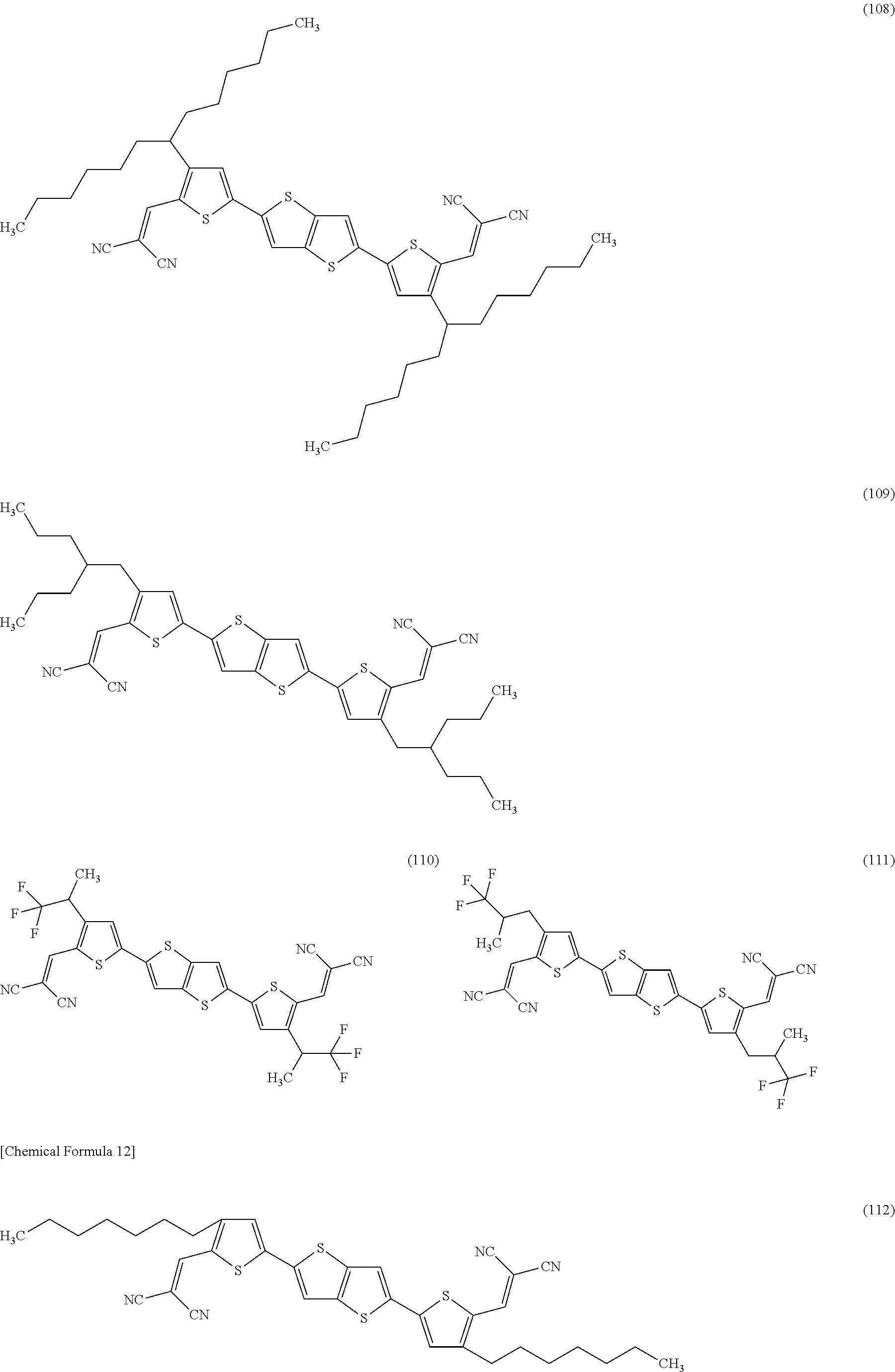

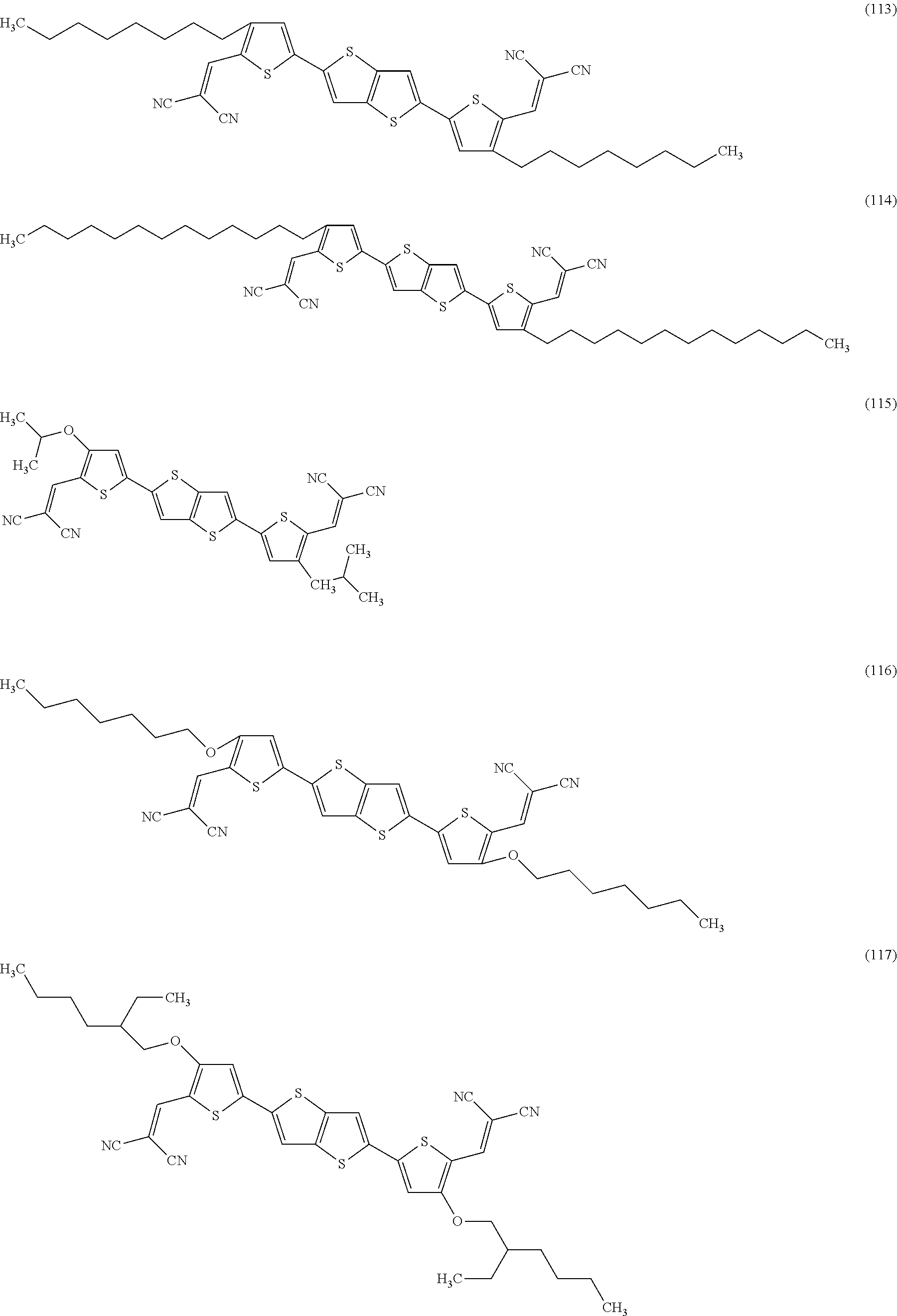

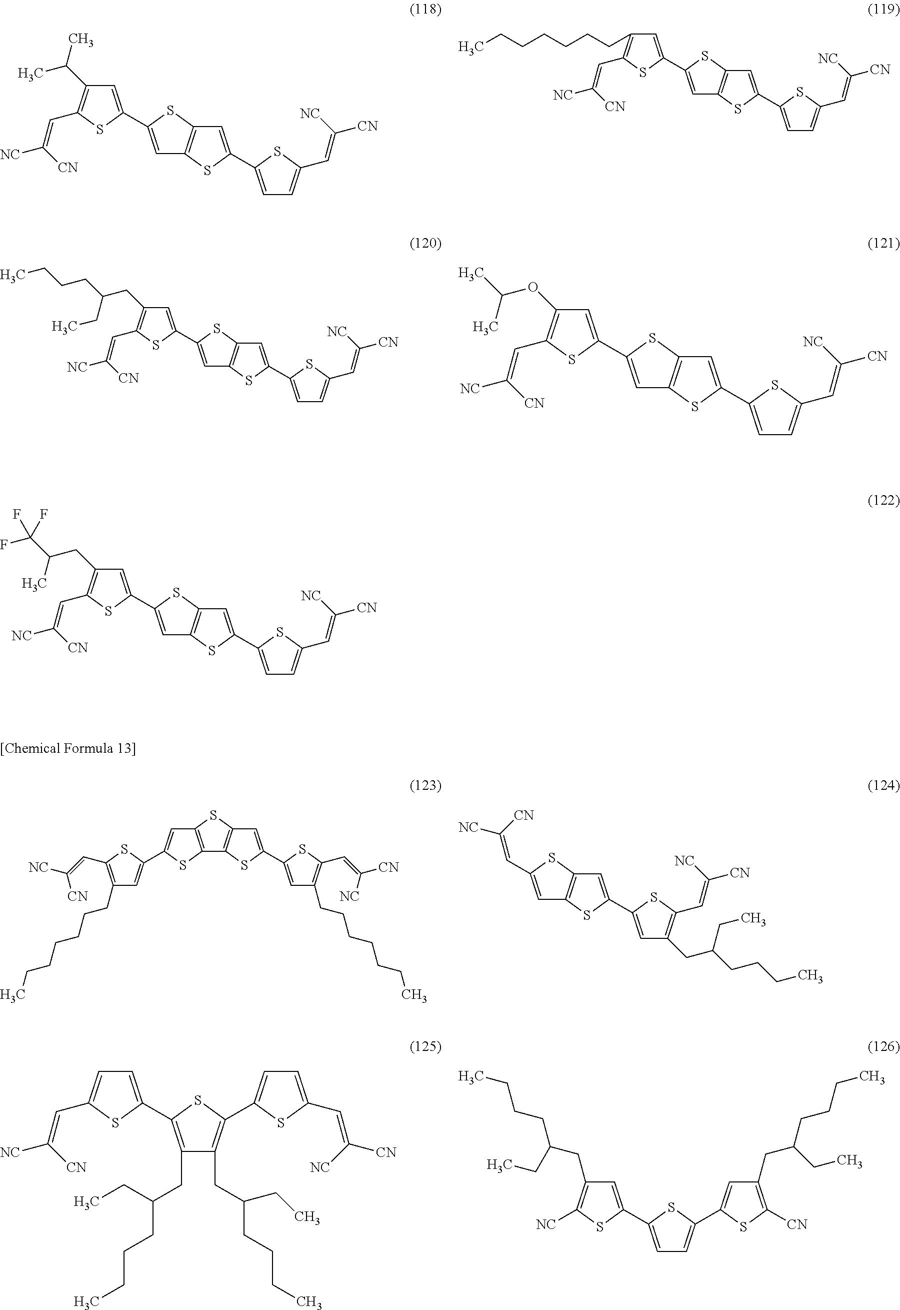

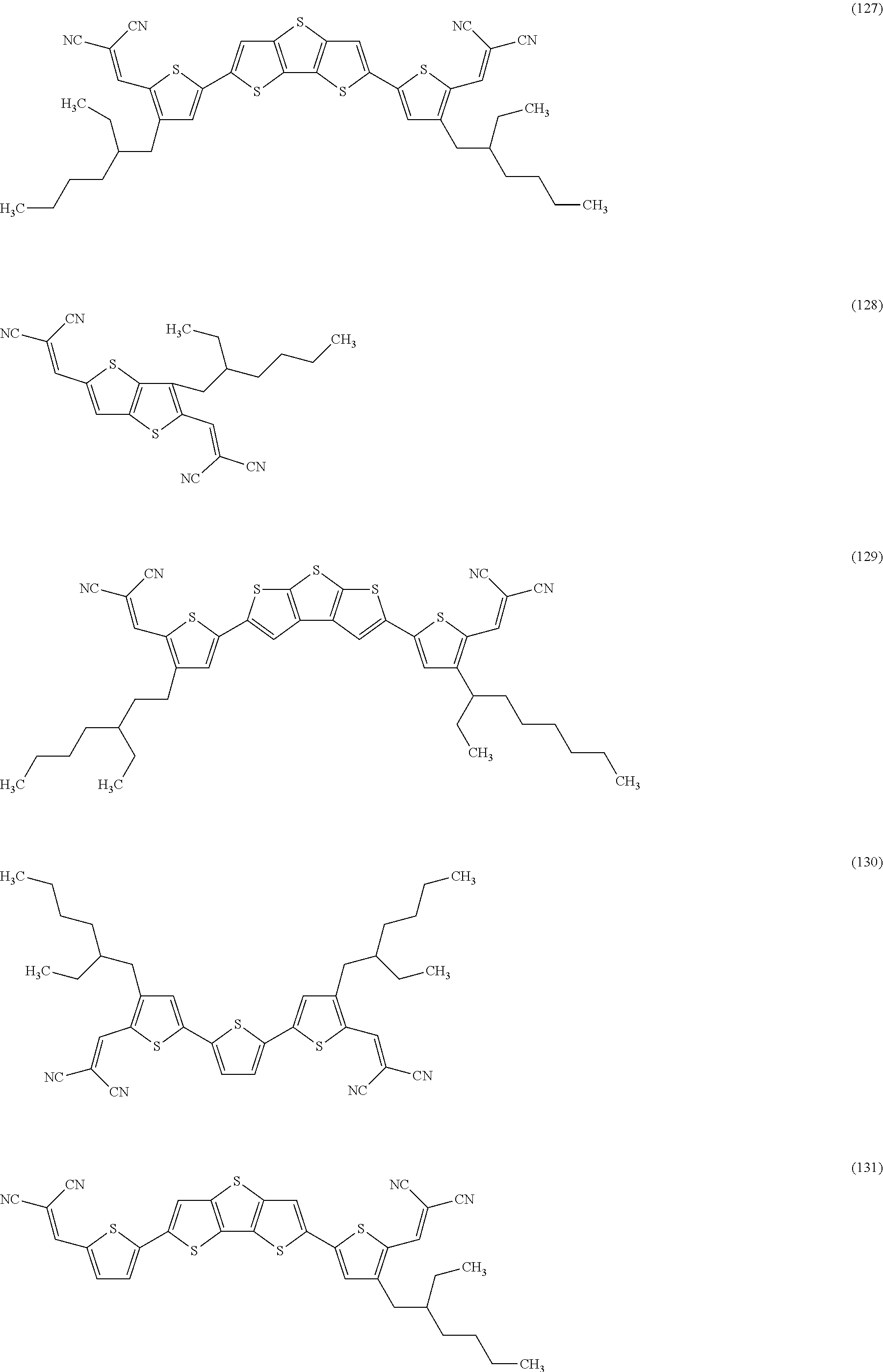

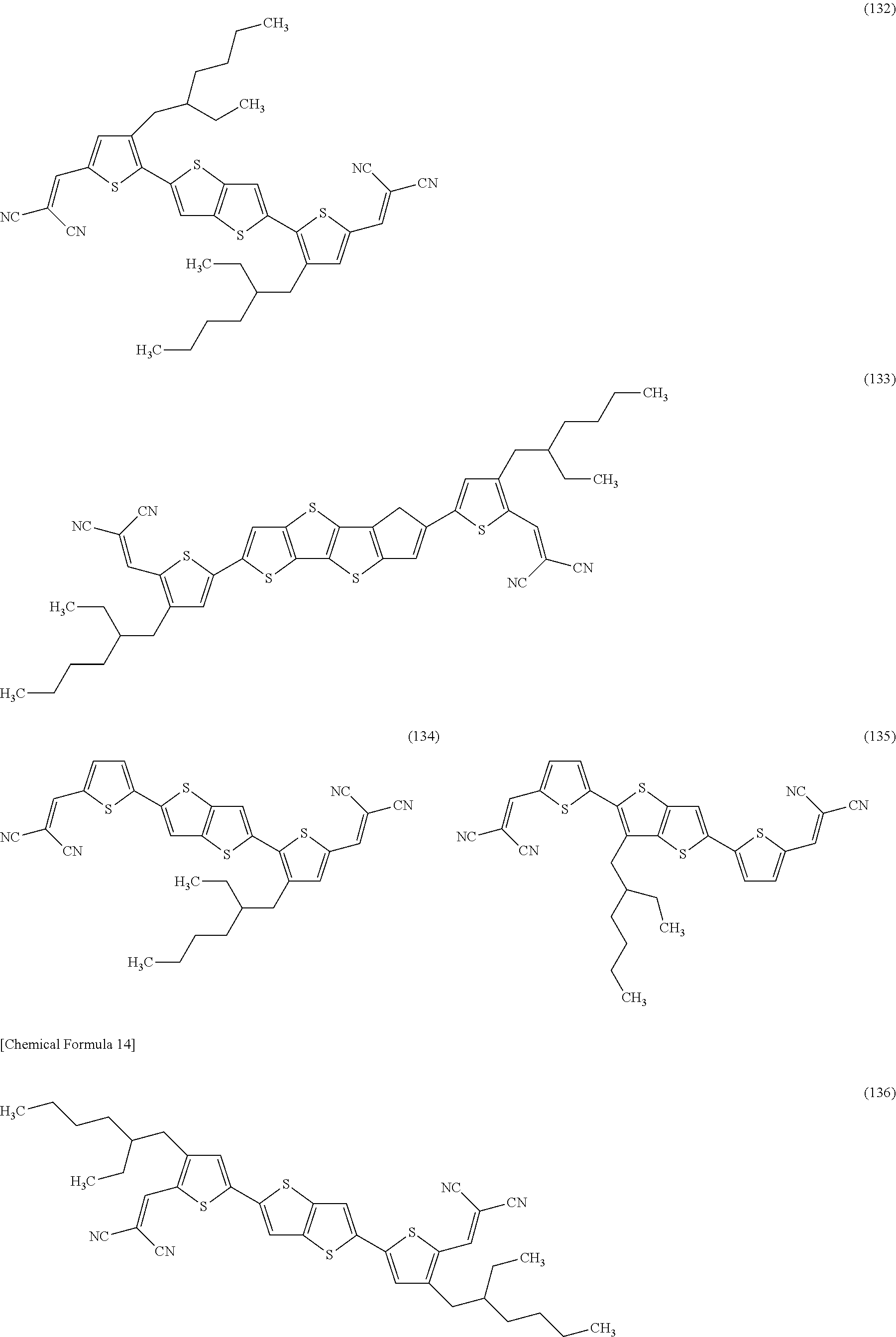

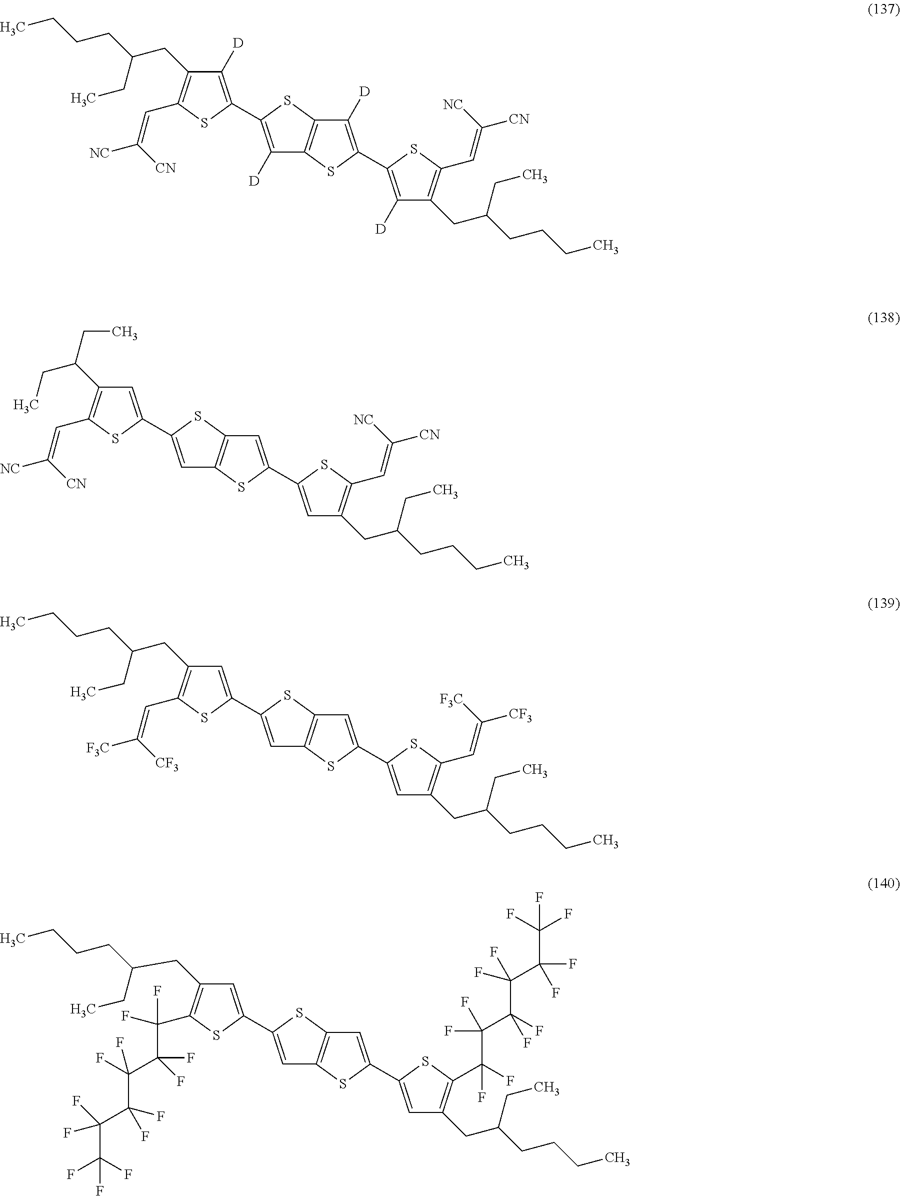

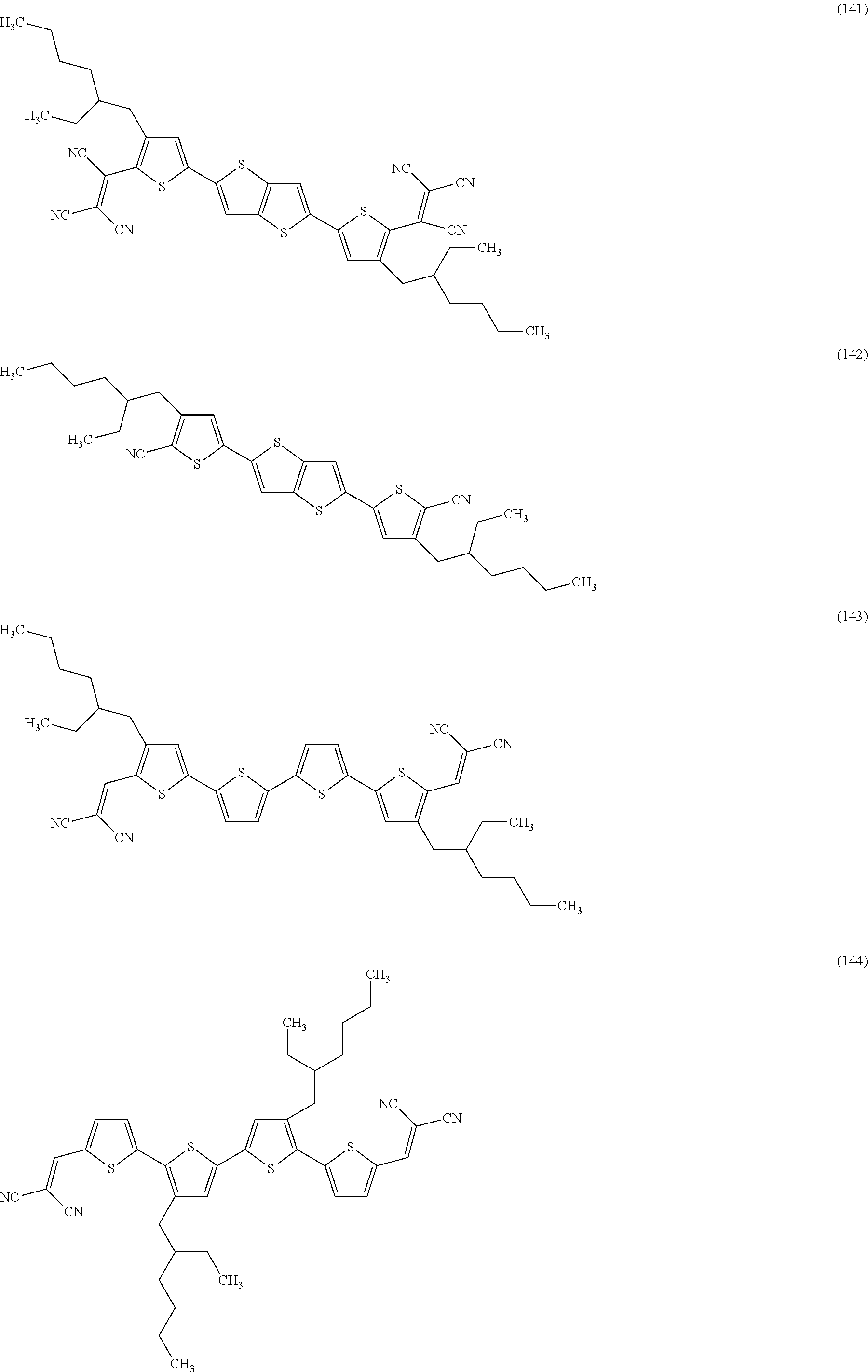

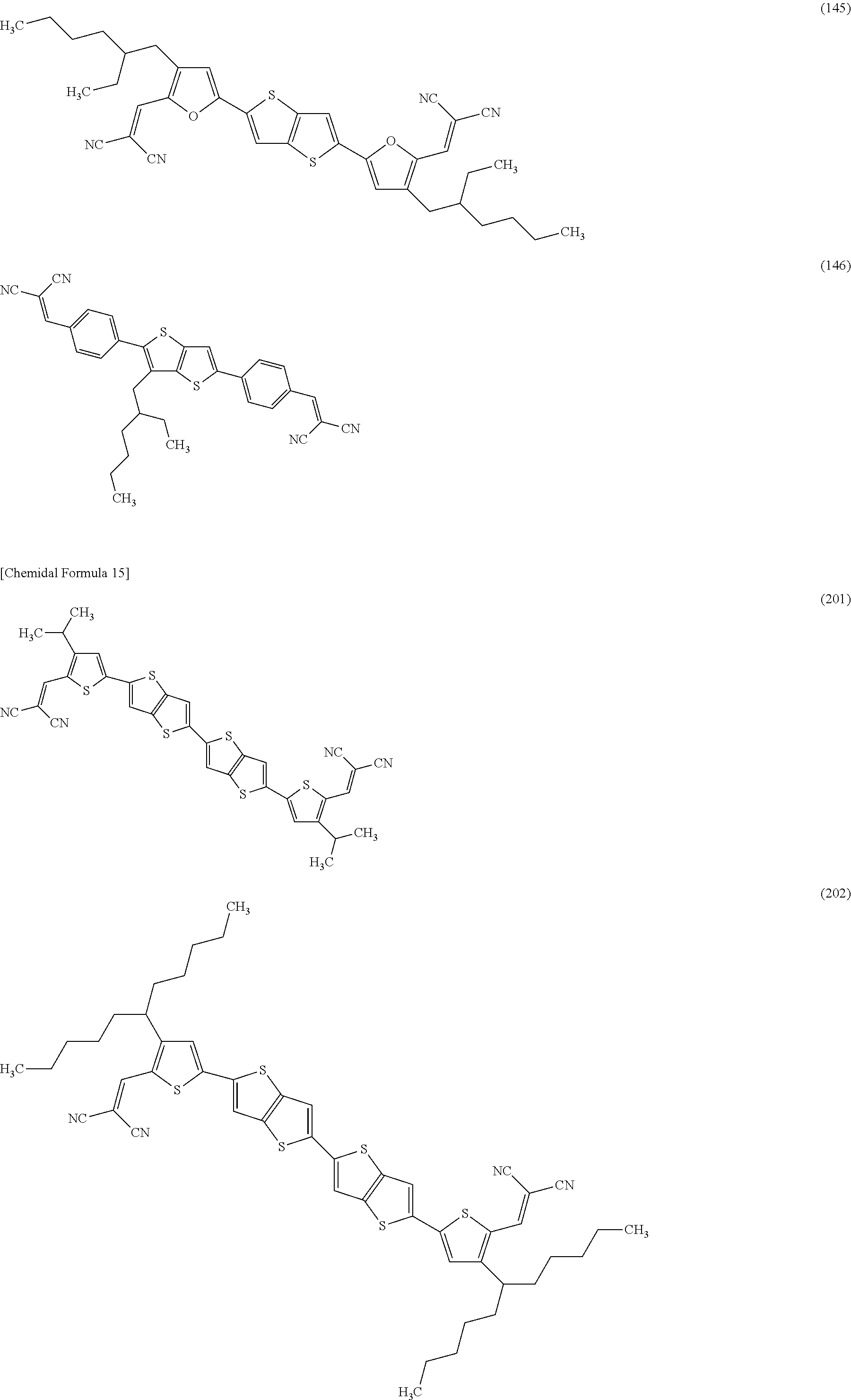

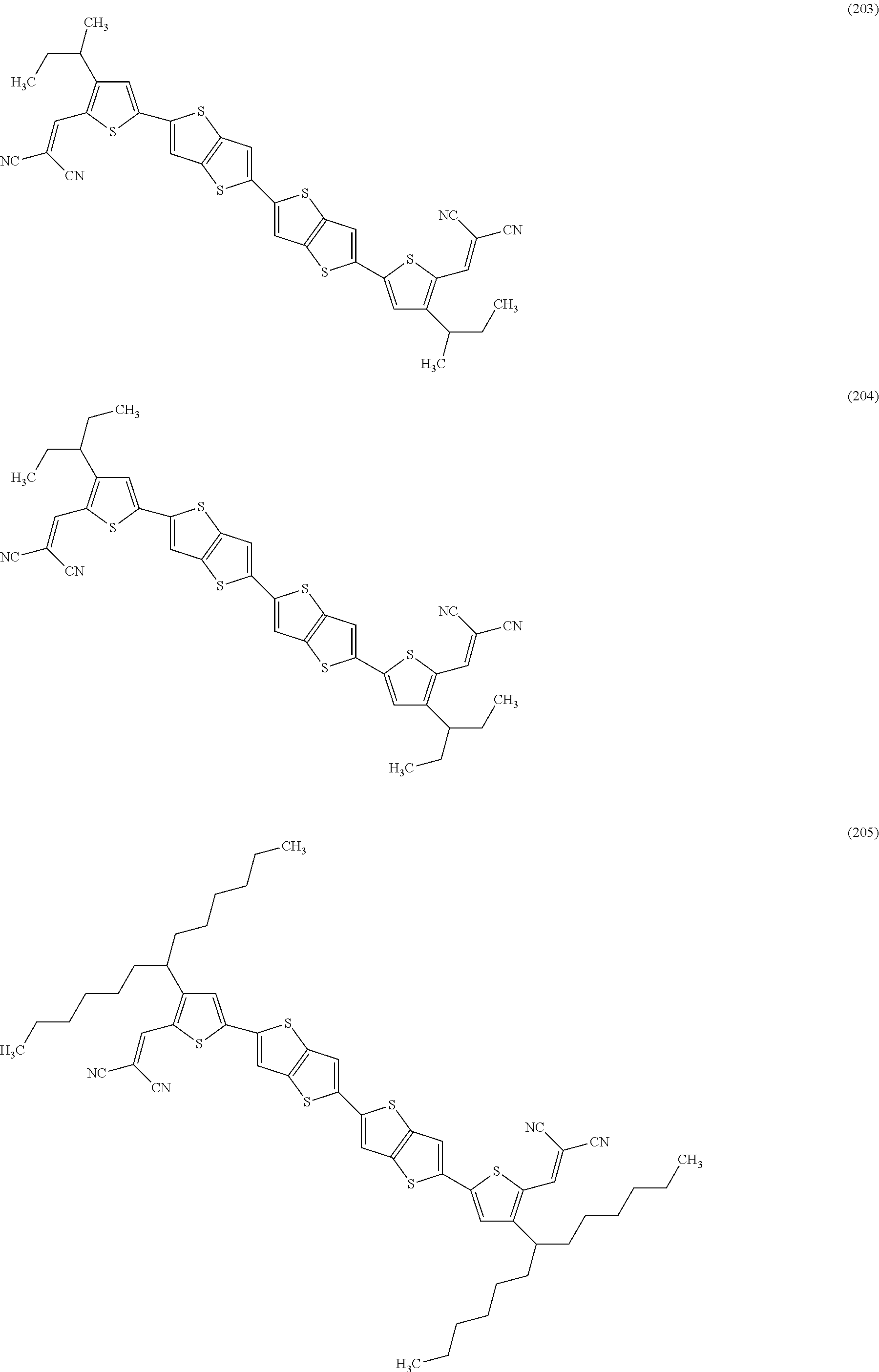 [SMILES: CCC(C)c1cc(-c2cc3sc(-c4cc5sc(-c6cc(C(C)CC)c(C=C(C#N)C#N)s6)cc5s4)cc3s2)sc1C=C(C#N)C#N ; CCC(CC)c1cc(-c2cc3sc(-c4cc5sc(-c6cc(C(CC)CC)c(C=C(C#N)C#N)s6)cc5s4)cc3s2)sc1C=C(C#N)C#N ; CCCCCCC(CCCCCC)c1cc(-c2cc3sc(-c4cc5sc(-c6cc(C(CCCCCC)CCCCCC)c(C=C(C#N)C#N)s6)cc5s4)cc3s2)sc1C=C(C#N)C#N]

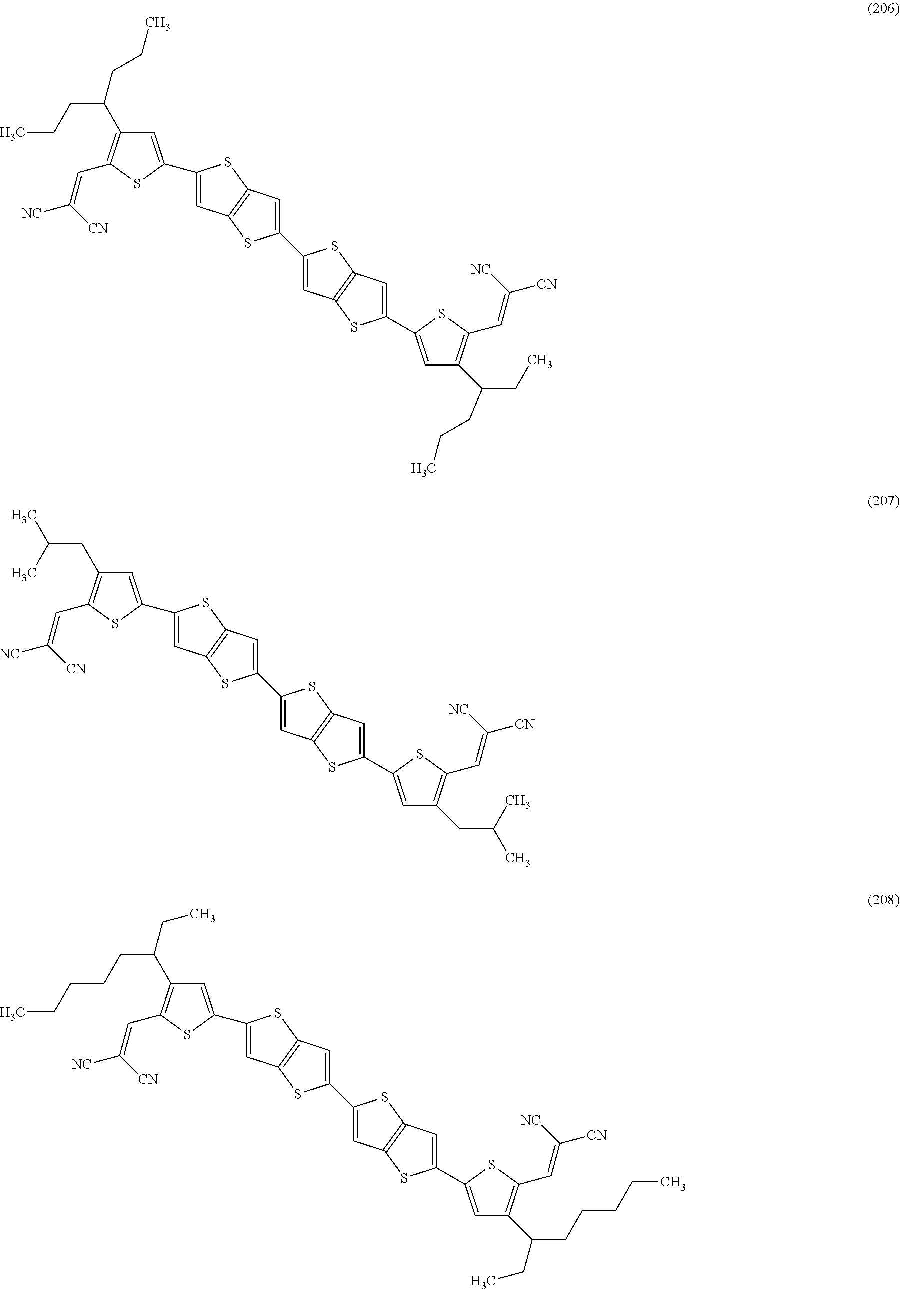 [SMILES: CCCC(CC)c1cc(-c2cc3sc(-c4cc5sc(-c6cc(C(CCC)CCC)c(C=C(C#N)C#N)s6)cc5s4)cc3s2)sc1C=C(C#N)C#N ; CC(C)Cc1cc(-c2cc3sc(-c4cc5sc(-c6cc(CC(C)C)c(C=C(C#N)C#N)s6)cc5s4)cc3s2)sc1C=C(C#N)C#N ; CCCCCC(CC)c1cc(-c2cc3sc(-c4cc5sc(-c6cc(C(CC)CCCCC)c(C=C(C#N)C#N)s6)cc5s4)cc3s2)sc1C=C(C#N)C#N]

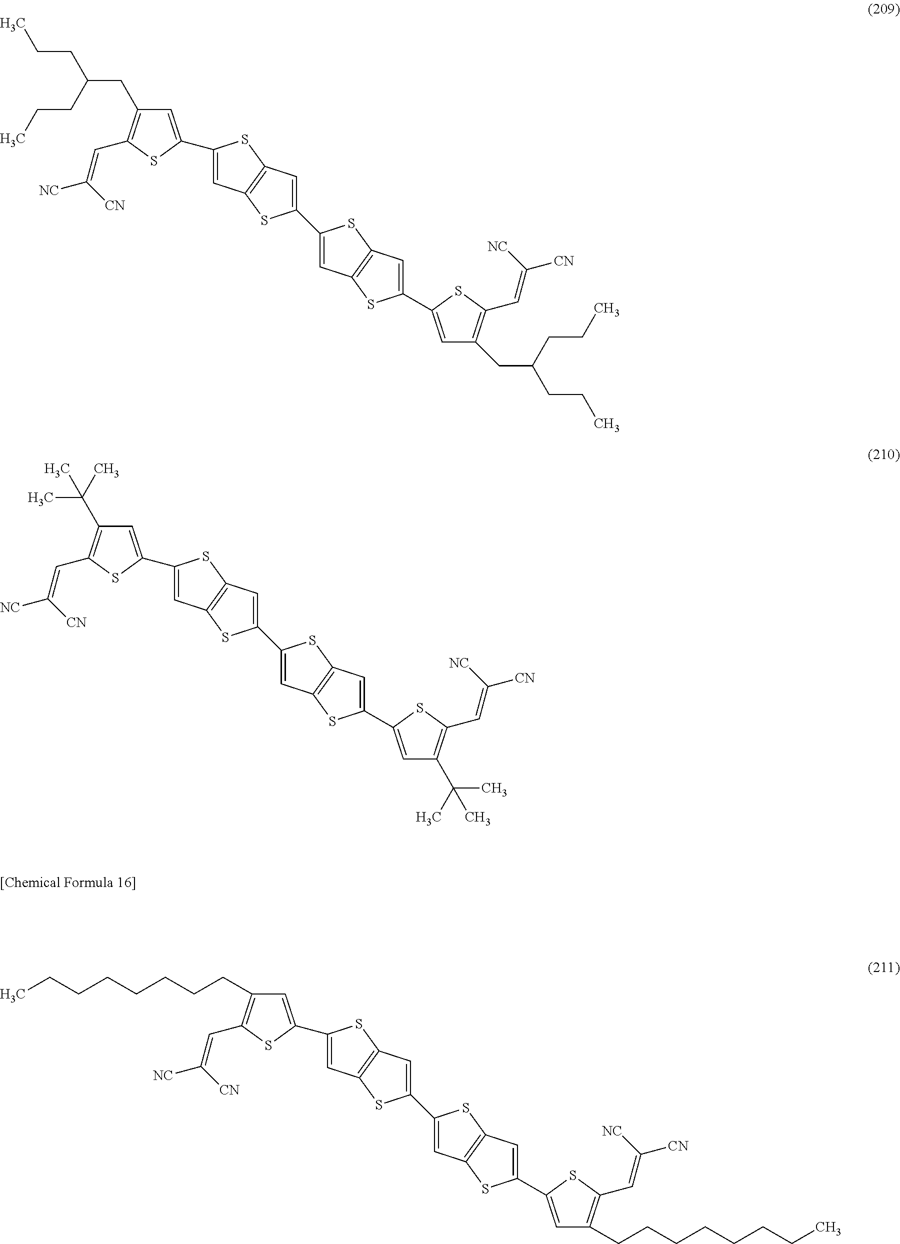

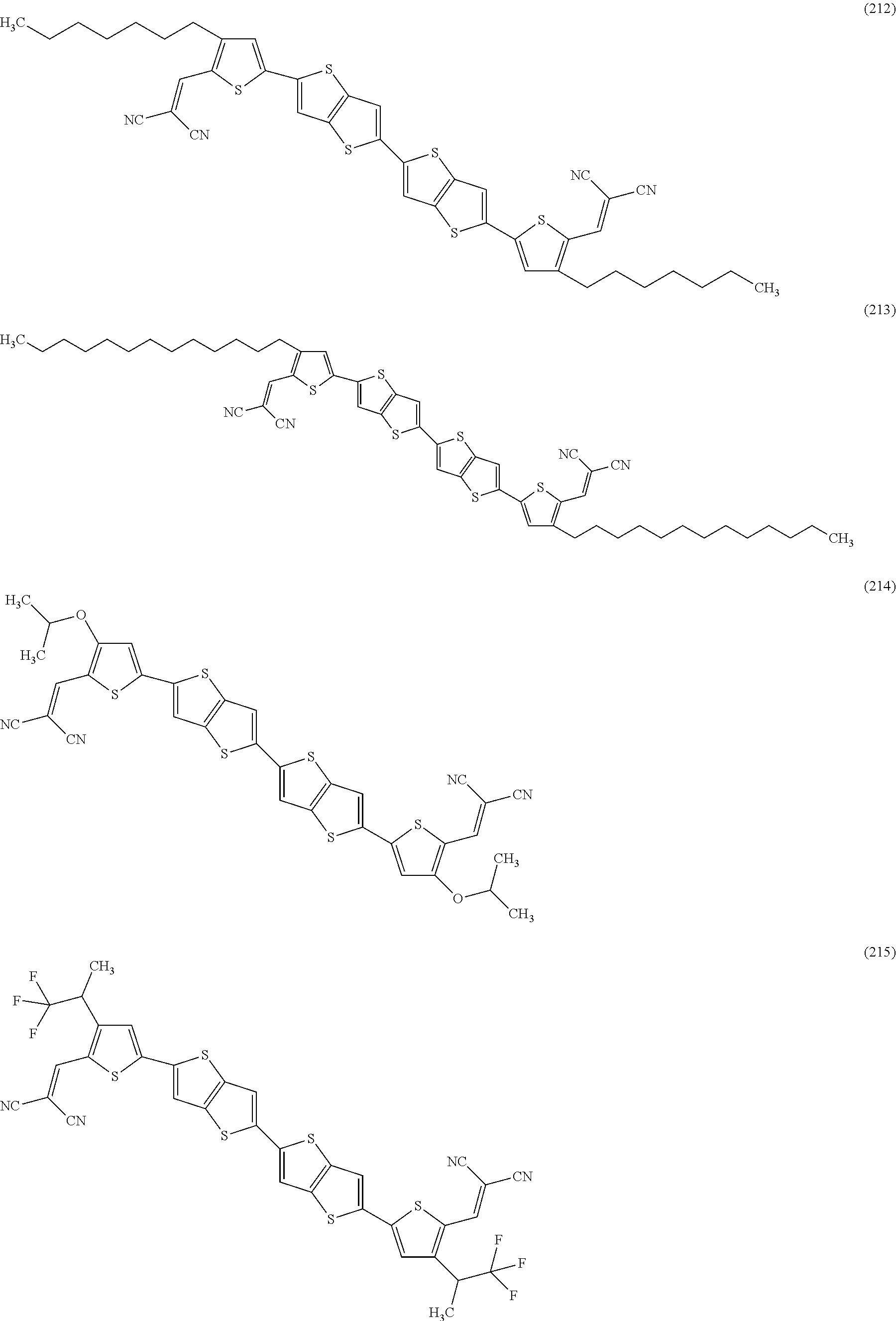

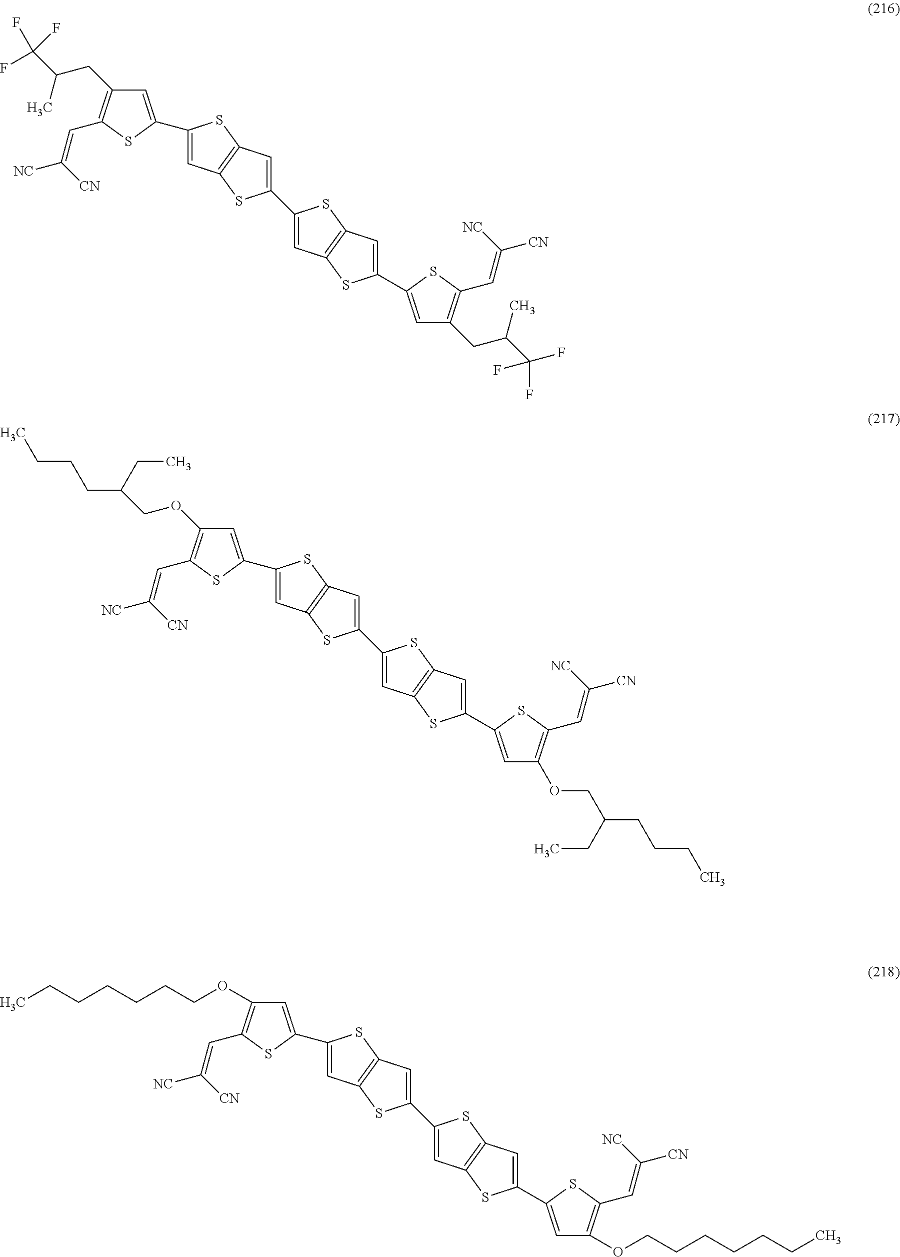

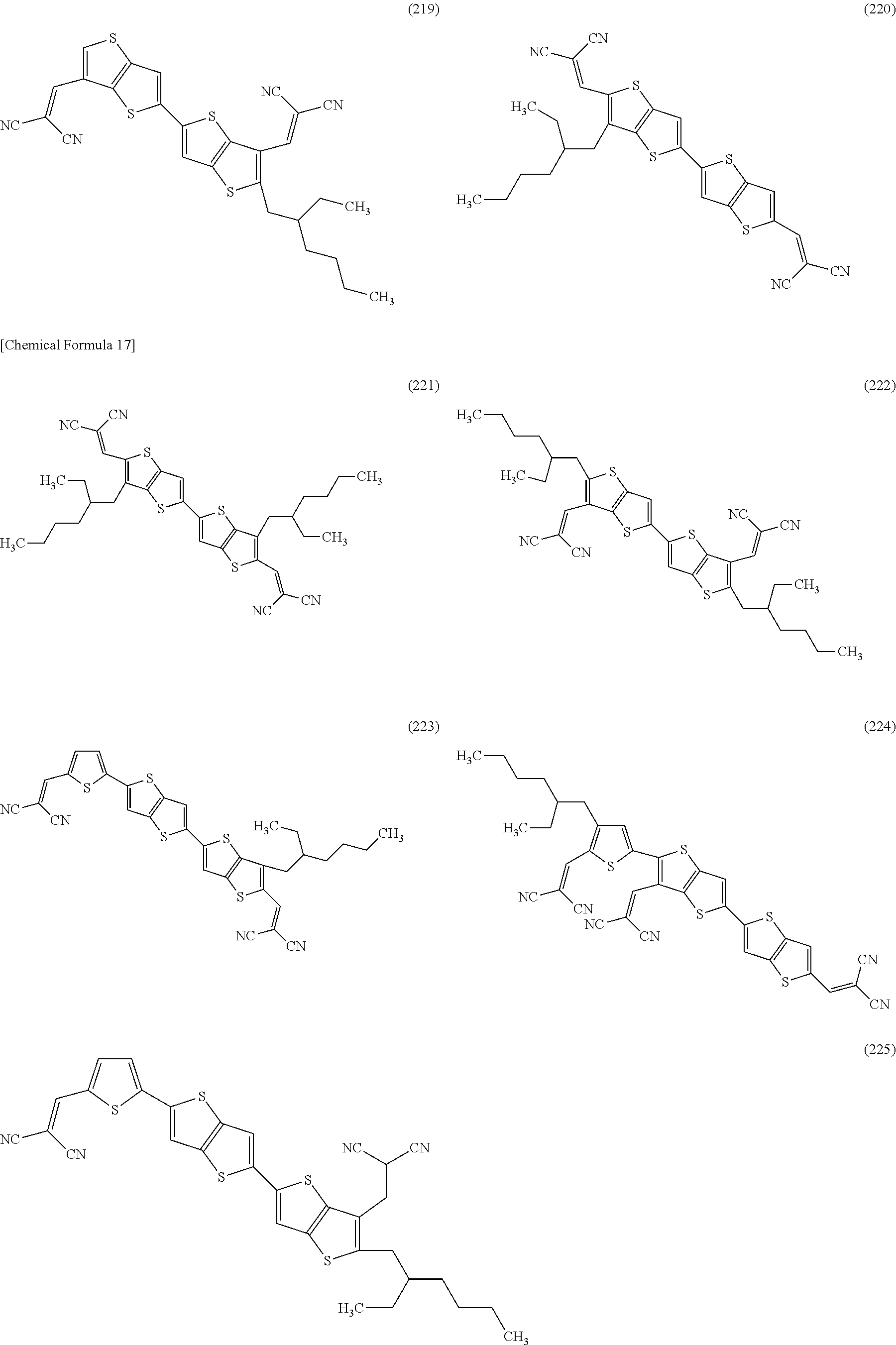

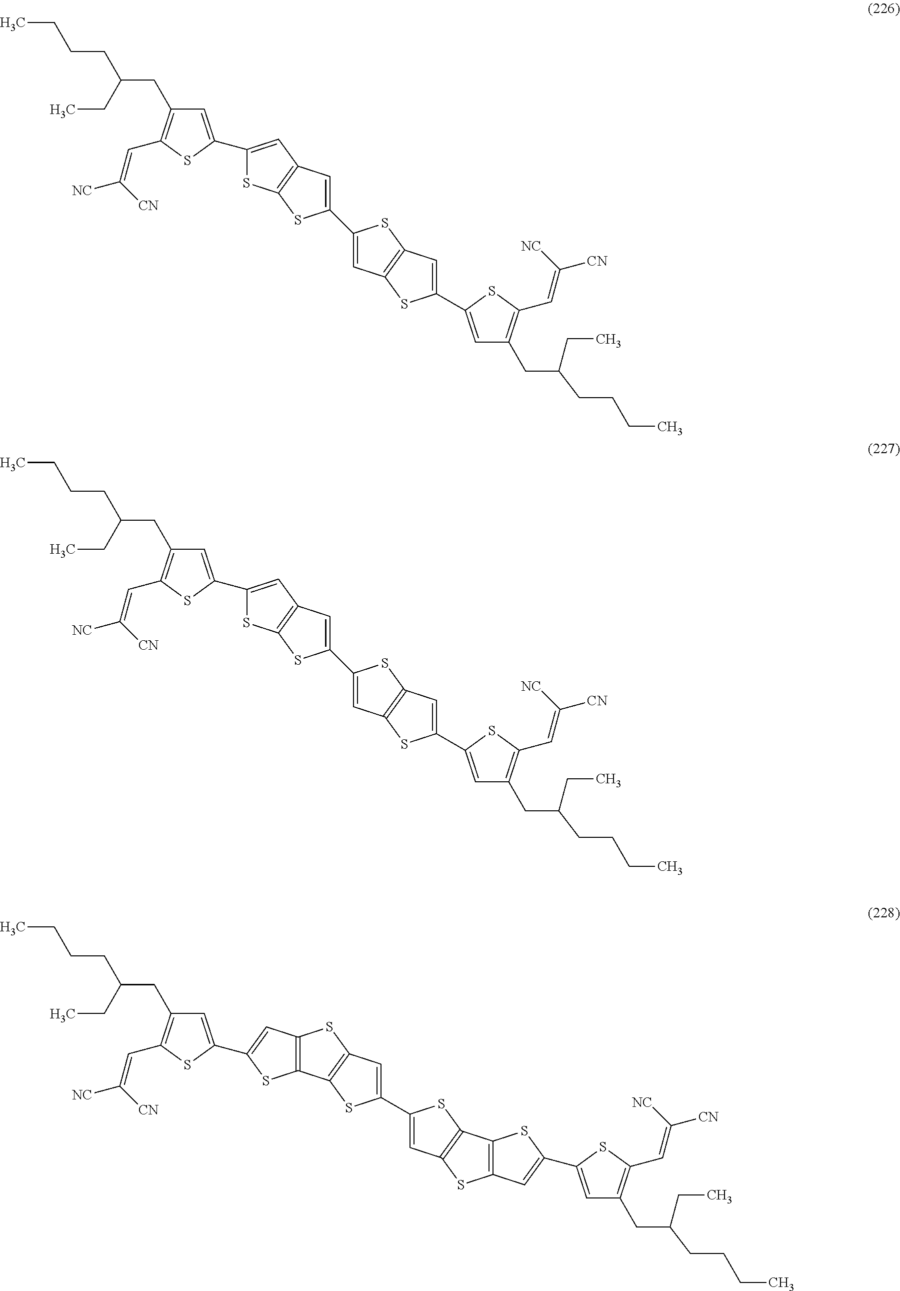

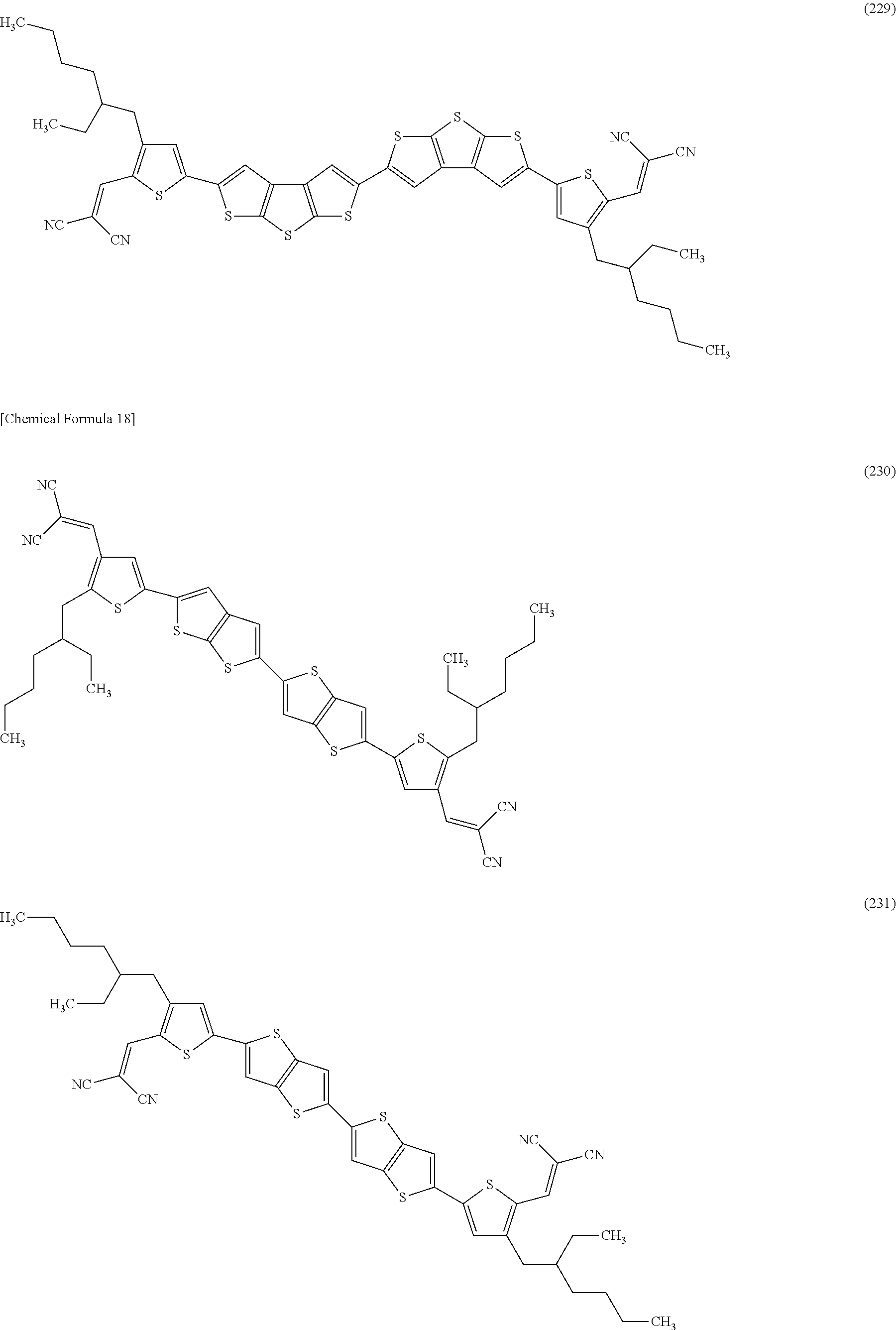

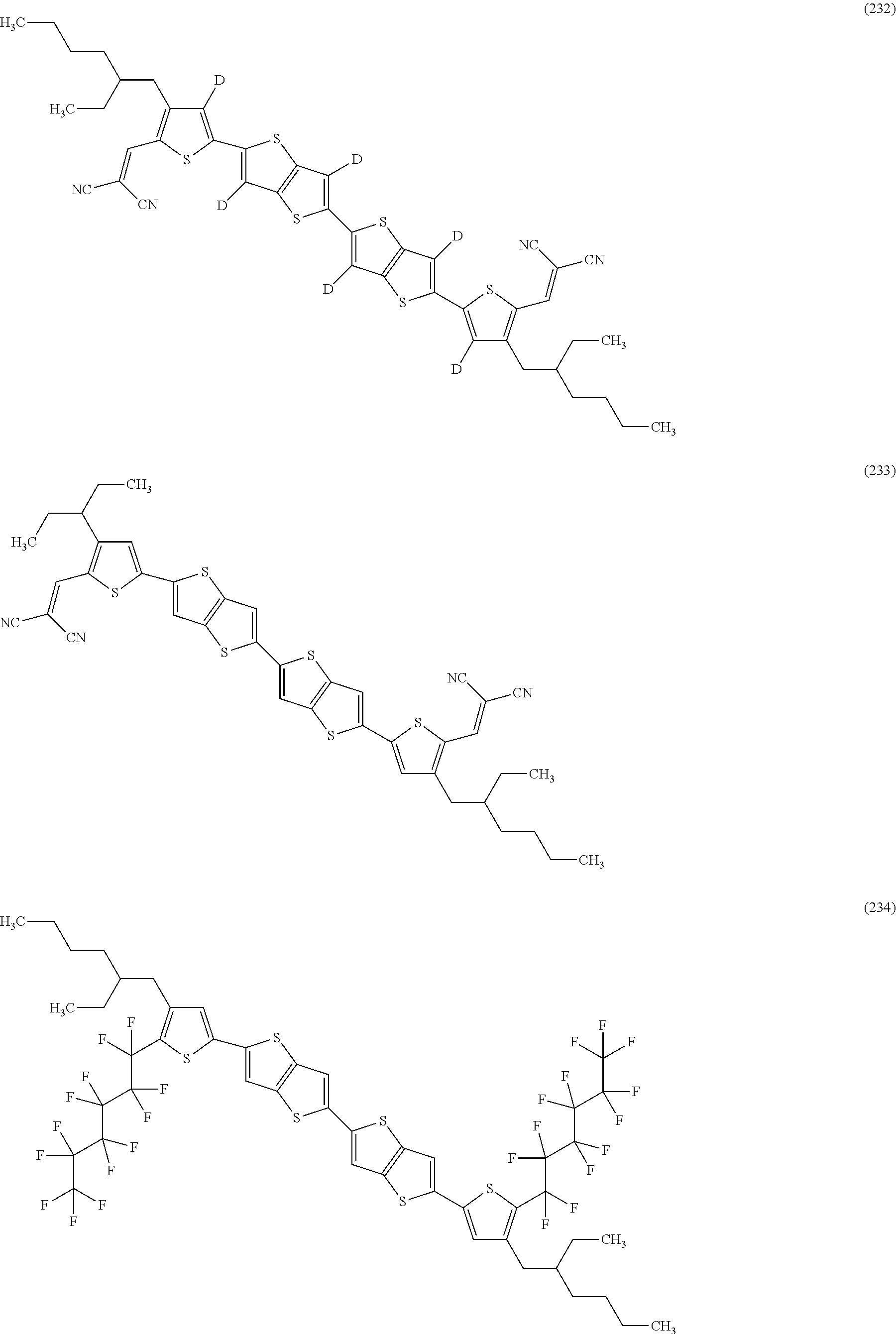

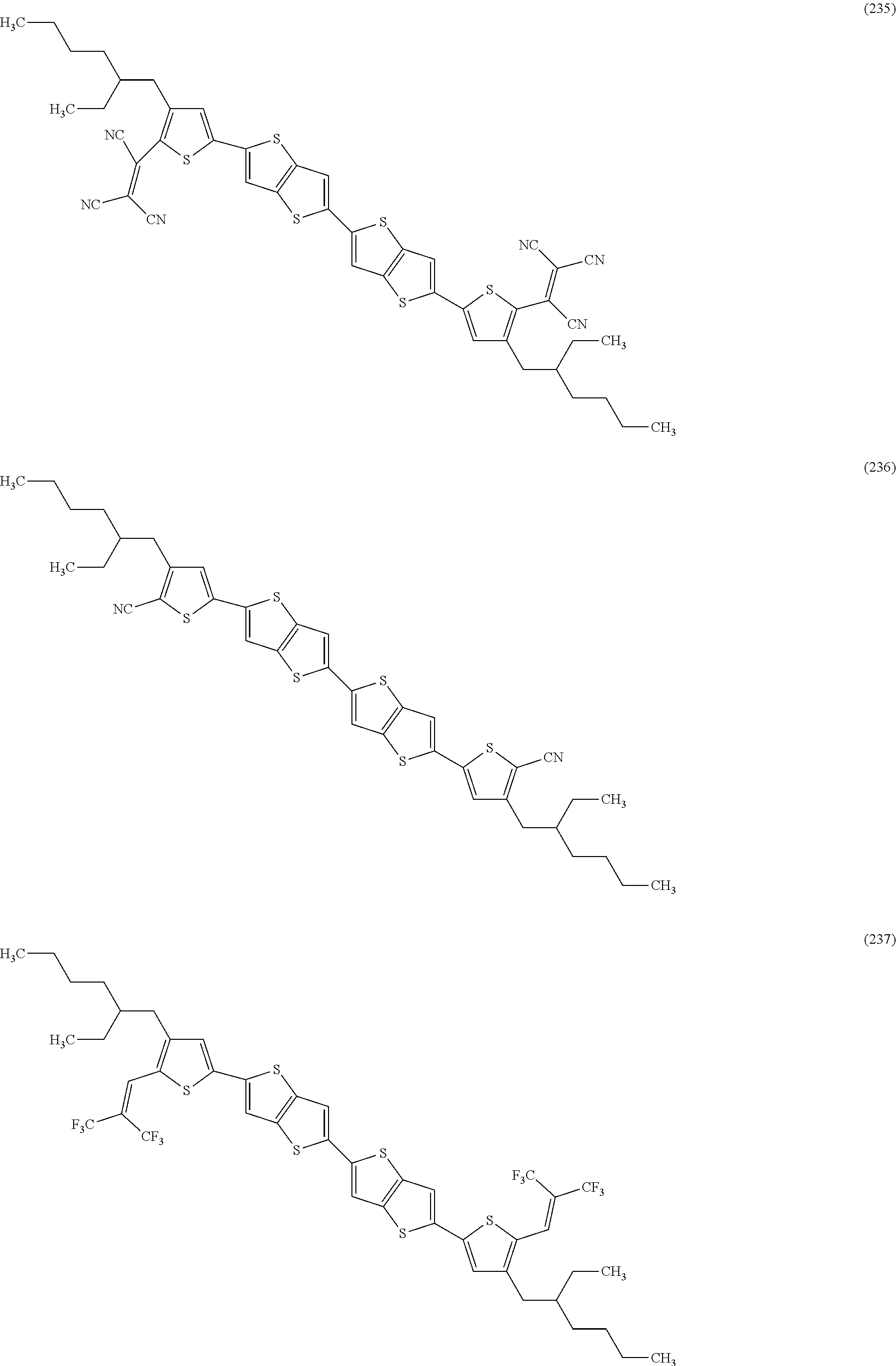

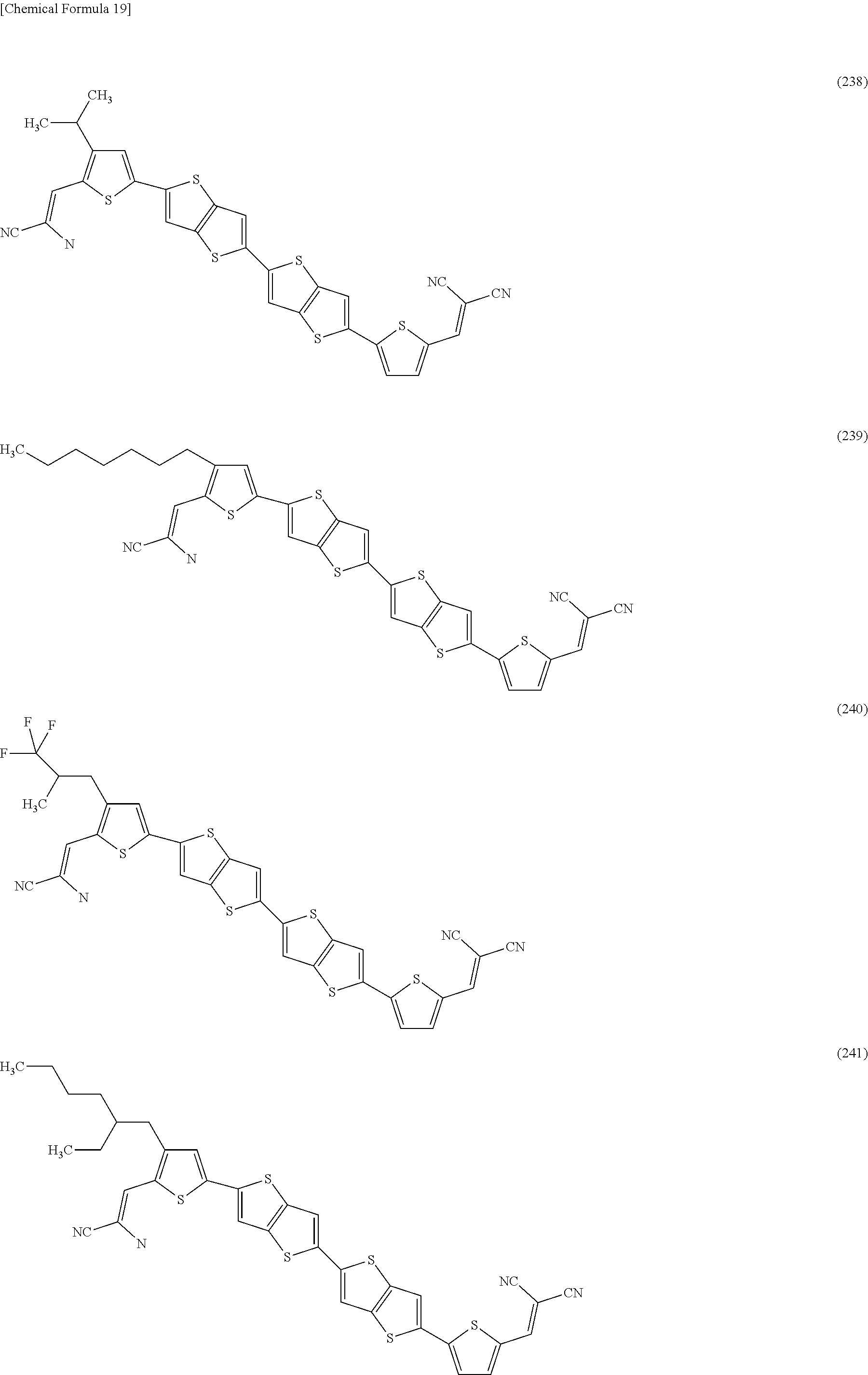 [SMILES: CC(C)c1cc(-c2cc3sc(-c4cc5sc(-c6ccc(C=C(C#N)C#N)s6)cc5s4)cc3s2)sc1/C=C(\N)C#N ; CCCCCCCc1cc(-c2cc3sc(-c4cc5sc(-c6ccc(C=C(C#N)C#N)s6)cc5s4)cc3s2)sc1/C=C(\N)C#N ; CC(Cc1cc(-c2cc3sc(-c4cc5sc(-c6ccc(C=C(C#N)C#N)s6)cc5s4)cc3s2)sc1/C=C(\N)C#N)C(F)(F)F ; CCCCC(CC)Cc1cc(-c2cc3sc(-c4cc5sc(-c6ccc(C=C(C#N)C#N)s6)cc5s4)cc3s2)sc1/C=C(\N)C#N]

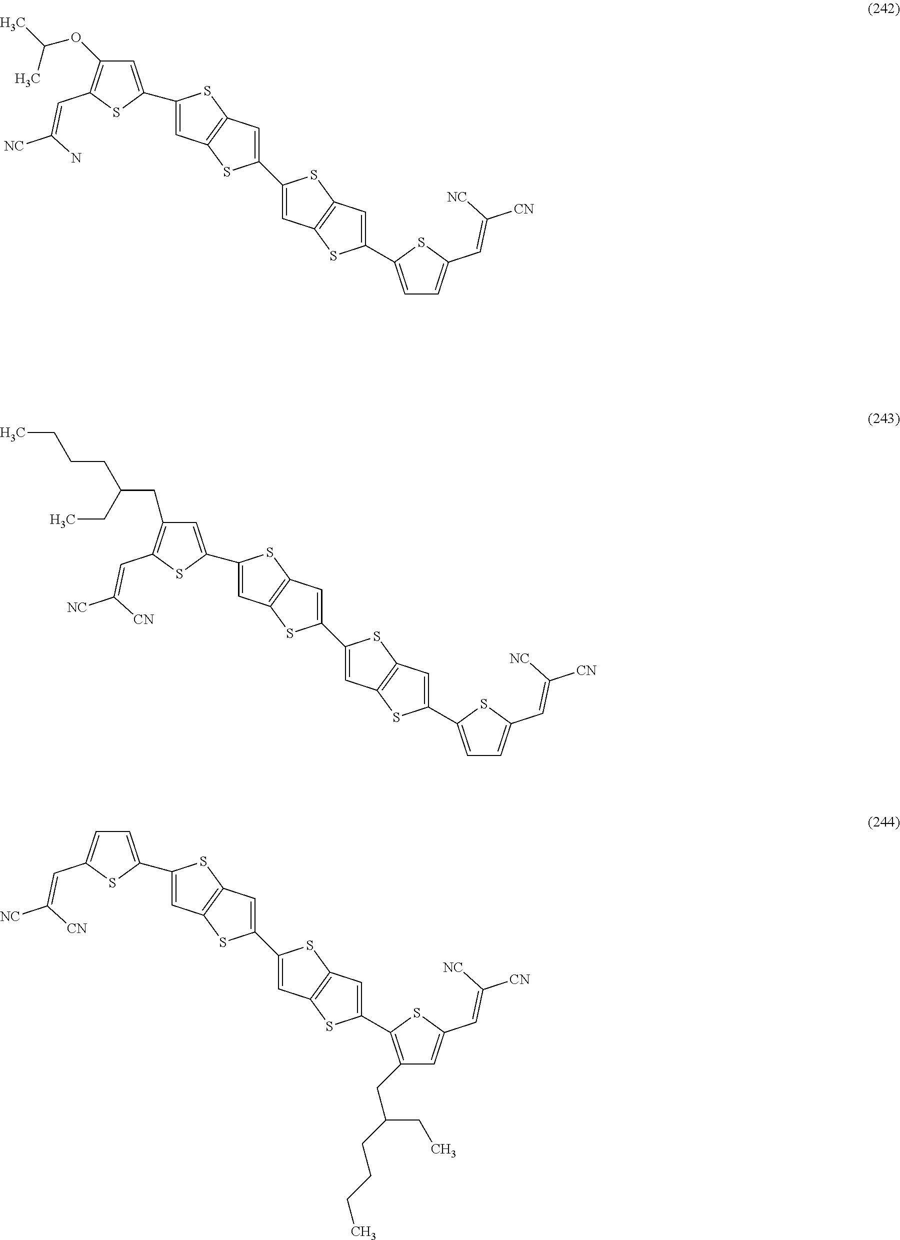 [SMILES: CC(C)Oc1cc(-c2cc3sc(-c4cc5sc(-c6ccc(C=C(C#N)C#N)s6)cc5s4)cc3s2)sc1/C=C(\N)C#N ; CCCCC(CC)Cc1cc(-c2cc3sc(-c4cc5sc(-c6ccc(C=C(C#N)C#N)s6)cc5s4)cc3s2)sc1C=C(C#N)C#N ; CCCCC(CC)Cc1cc(C=C(C#N)C#N)sc1-c1cc2sc(-c3cc4sc(-c5ccc(C=C(C#N)C#N)s5)cc4s3)cc2s1]

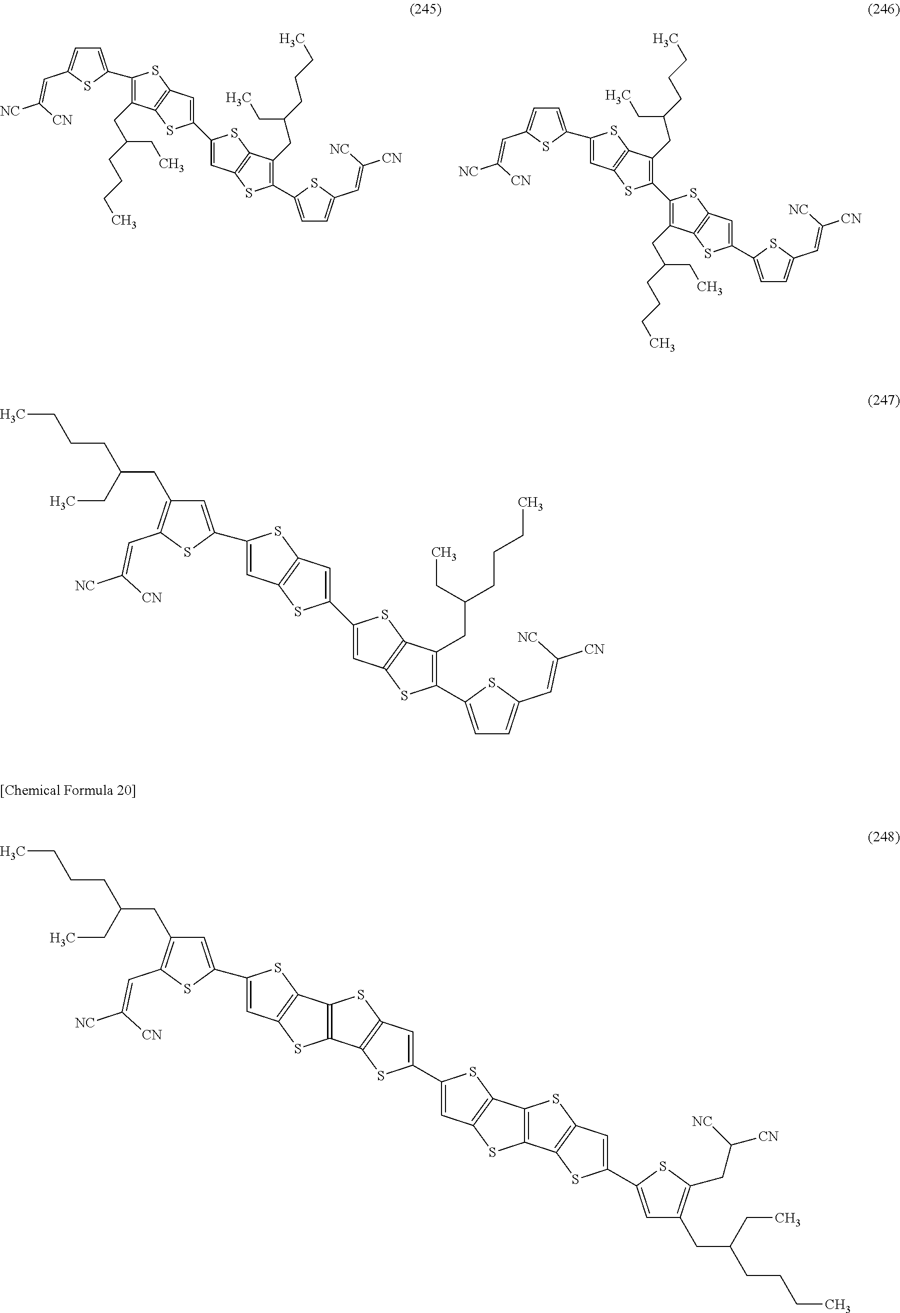 [SMILES: CCCCC(CC)Cc1c(-c2ccc(C=C(C#N)C#N)s2)sc2cc(-c3cc4sc(-c5ccc(C=C(C#N)C#N)s5)c(CC(CC)CCCC)c4s3)sc12 ; CCCCC(CC)Cc1c(-c2sc3cc(-c4ccc(C=C(C#N)C#N)s4)sc3c2CC(CC)CCCC)sc2cc(-c3ccc(C=C(C#N)C#N)s3)sc12 ; CCCCC(CC)Cc1cc(-c2cc3sc(-c4cc5sc(-c6ccc(C=C(C#N)C#N)s6)c(CC(CC)CCCC)c5s4)cc3s2)sc1C=C(C#N)C#N ; CCCCC(CC)Cc1cc(-c2cc3sc4c5sc(-c6cc7sc8c9sc(-c%10cc(CC(CC)CCCC)c(CC(C#N)C#N)s%10)cc9sc8c7s6)cc5sc4c3s2)sc1C=C(C#N)C#N]

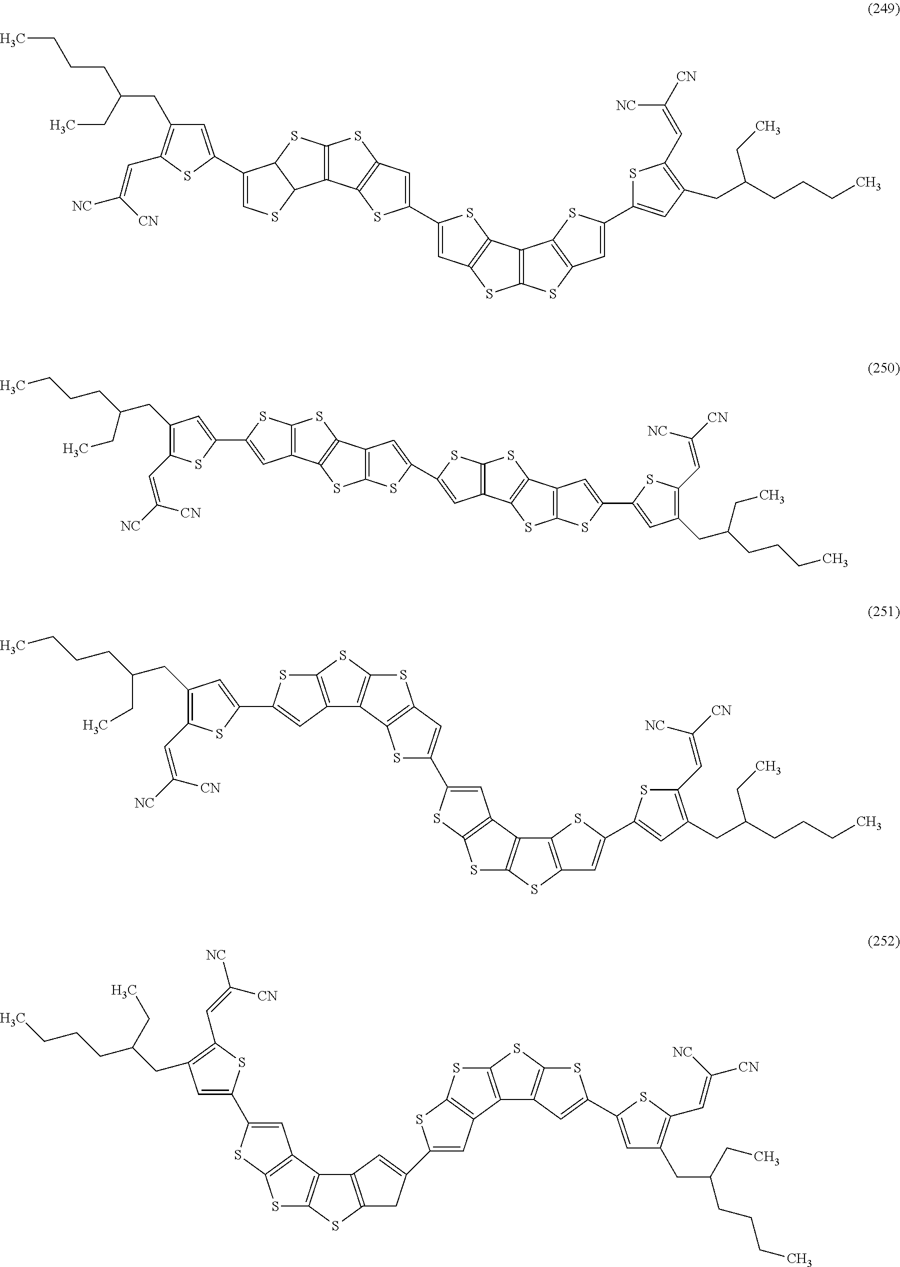

### Synthesis Method of Organic Compound

A synthesis method of the organic compound of one embodiment of the present invention is described using reaction schemes shown below. Here, synthesis methods of the organic compounds represented by General Formulas (G1), (G1-1a-1), and (G1-1a-2) are described.

In General Formula (G1-1a-1), n₁₄ represents 0 and n₁₅ to n₁₇ represent 1. In General Formula (G1-1a-2), n₁₄ represents 0, n₁₅ and n₁₆ represent 1, and n₁₇ represents 2. In the above-described general formulas, R¹²¹ to R¹³², R¹⁶⁶, and R¹⁶⁷ each independently represent hydrogen, deuterium, a branched alkyl group having 3 to 20 carbon atoms, a straight-chain alkyl group having 7 or more carbon atoms, a cycloalkyl group having 3 to 10 carbon atoms, a branched alkoxy group having 3 to 20 carbon atoms, a straight-chain alkoxy group having 7 or more carbon atoms, a substituted or unsubstituted aryl group having 6 to 30 carbon atoms, a substituted or unsubstituted heteroaryl group having 2 to 30 carbon atoms, a branched alkyl halide group having 3 to 20 carbon atoms, a straight-chain alkyl halide group having 7 or more carbon atoms, or a halogen. Furthermore, X¹⁵, X¹⁸, X²⁹, X³⁰, X³¹, and X³² each independently represent oxygen or sulfur. An organic compound of another embodiment of the present invention can also be synthesized by the same method when a raw material having the substituents at the corresponding substitution sites is used.

The description of General Formula (G1) above can apply to D¹, the substituents Ar¹ and Ar², the substituents A¹ and A², the substituents R¹²¹ to R¹³², X¹⁵, X¹⁸, X²⁹, X³⁰, the substituents R¹⁶⁶ and R¹⁶⁷, X³¹, and X³² in General Formulas (G1), (G1-1a-1), and (G1-1a-2), Reaction Schemes (s1-1) to (s1-16), (s2-1) to (s2-8), and (s3-1) to (s3-4); therefore, the description thereof is omitted.

### Synthesis Method 1 of Organic Compound Represented by General Formula (G1)

An example of the synthesis method of the organic compound represented by General Formula (G1) is described below.

[Chemical Formula 21]

A¹-Ar¹-D¹-Ar²-A² (G1)

A variety of reactions can be applied to the synthesis method of the organic compound represented by General Formula (G1). For example, synthesis reactions described below enable the synthesis of the organic compound represented by General Formula (G1).

Specifically, the organic compound of the present invention represented by General Formula (G1) can be synthesized by Reaction Schemes (s1-1) to (s1-16) below.

First, Reaction Schemes (s1-1) and (s1-2) are described. The introduction of A¹ into an aryl compound (Compound 1) can form an A¹-substituted aryl compound (Compound 2), and then the introduction of a functional group into the aryl compound (Compound 2) can form a functional-group-Y¹-substituted aryl compound (Compound 3). Reaction Schemes (s1-1) and (s1-2) are shown below. Note that a functional group is a substituent that can serve as a reactive site in a molecule.

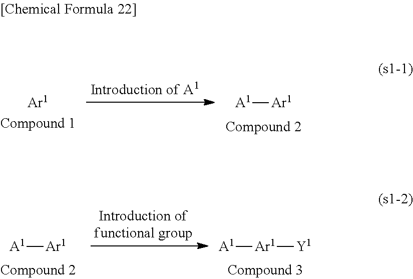

The aryl compound (Compound 3) can also be obtained by Reaction Schemes (s1-3) and (s1-4). The introduction of a functional group into the aryl compound (Compound 1) can form a functional-group-Y¹-substituted aryl compound (Compound 4), and then the introduction of A¹ into the aryl compound (Compound 4) can form the A¹-substituted aryl compound (Compound 3). Reaction Schemes (s1-3) and (s1-4) are shown below.

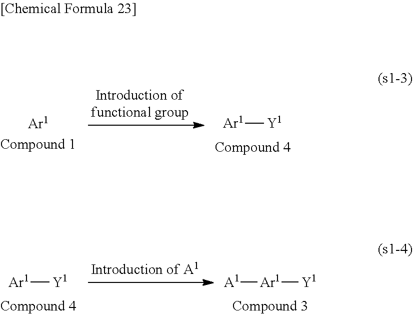

Next, Reaction Schemes (s1-5) and (s1-6) are described. The introduction of A² into an aryl compound (Compound 5) can form an A²-substituted aryl compound (Compound 6), and then the introduction of a functional group into the aryl compound (Compound 6) can form a functional-group-Y²-substituted aryl compound (Compound 7). Reaction Schemes (s1-5) and (s1-6) are shown below.

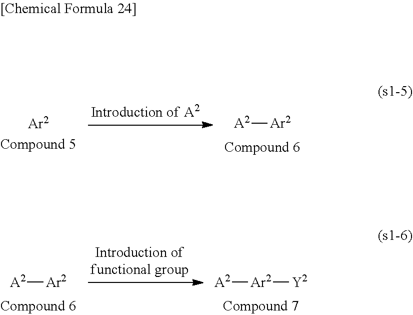

The aryl compound (Compound 7) can also be obtained by Reaction Schemes (s1-7) and (s1-8). The introduction of a functional group into the aryl compound (Compound 5) can form a functional-group-Y²-substituted aryl compound (Compound 8), and then the introduction of A¹ into the aryl compound (Compound 8) can form the A¹-substituted aryl compound (Compound 7). Reaction Schemes (s1-7) and (s1-8) are shown below.

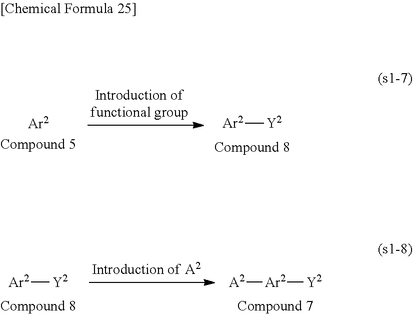

Next, Reaction Schemes (s1-9) and (s1-10) are described. The introduction of a functional group into an aryl compound (Compound 9) can form a functional-group-Y³-substituted aryl compound (Compound 10), and then the introduction of a functional group into the aryl compound (Compound 10) can form a functional-group-Y⁴-substituted aryl compound (Compound 11). Reaction Schemes (s1-9) and (s1-10) are shown below.

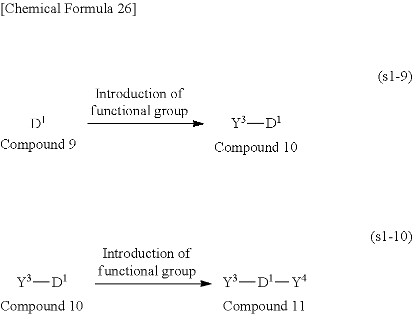

Note that in the case where Y³ and Y⁴ are the same functional group, the functional group may be introduced into one equivalent of the aryl compound (Compound 9) using two equivalents of reagent, whereby the aryl compound (Compound 11) can be obtained from the aryl compound (Compound 9) in one step.

Next, Reaction Schemes (s1-11) and (s1-12) are described. Coupling between the aryl compound (Compound 11) and the aryl compound (Compound 3) can form an aryl compound (Compound 12), and then coupling between the aryl compound (Compound 12) and the aryl compound (Compound 7) can form the organic compound represented by General Formula (G1), which is the objective substance. Reaction Schemes (s1-11) and (s1-12) are shown below.

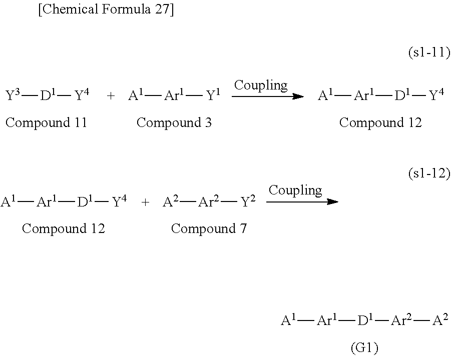

Note that in the case where the aryl compound (Compound 3) and the aryl compound (Compound 7) have the same structure, coupling between one equivalent of the aryl compound (Compound 11) and two equivalents of the aryl compound (Compound 3 or 7) can form the organic compound represented by General Formula (G1), which is the objective substance, from the aryl compound (Compound 11) in one step.

### Synthesis Method 2 of Organic Compound Represented by General Formula (G1)

An example of the synthesis method of the organic compound represented by General Formula (G1) is described below. The organic compound represented by General Formula (G1), which is the objective substance, can also be obtained by Reaction Schemes (s1-13) to (s1-16).

First, Reaction Schemes (s1-13) and (s1-14) are described. Coupling between an aryl compound (Compound 13) and the aryl compound (Compound 4) can form an aryl compound (Compound 14), and then coupling between the aryl compound (Compound 14) and the aryl compound (Compound 8) can form an aryl compound (Compound 15). Reaction Schemes (s1-13) and (s1-14) are shown below.

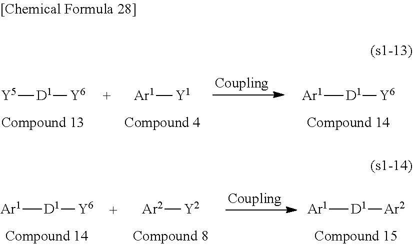

Note that in the case where the aryl compound (Compound 4) and the aryl compound (Compound 8) have the same structure, coupling between one equivalent of the aryl compound (Compound 13) and two equivalents of the aryl compound (Compound 4 or 8) can form the aryl compound (Compound 15) from the aryl compound (Compound 13) in one step.

Next, Reaction Schemes (s1-15) and (s1-16) are described. The introduction of A¹ into the aryl compound (Compound 15) can form an A¹-substituted aryl compound (Compound 16), and then the introduction of A² into the aryl compound (Compound 16) can form the organic compound represented by General Formula (G1), which is the objective substance. Reaction Schemes (s1-15) and (s1-16) are shown below.

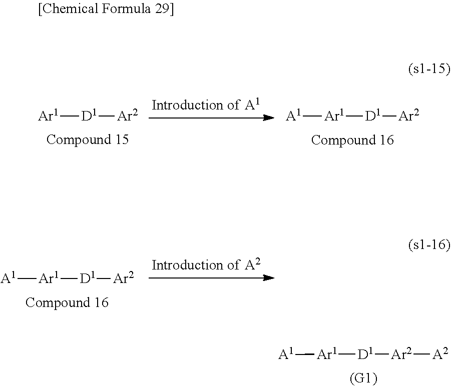

Note that in the case where A¹ and A² are the same substituent, the substituent may be introduced into the one equivalent of the aryl compound (Compound 15) using two equivalents of reagent corresponding to A¹ or A², whereby the organic compound represented by General Formula (G1), which is the objective substance, can be obtained from the aryl compound (Compound 15) in one step.

### Synthesis Method 1 of Organic Compound Represented by General Formula (G1-1a-1)

An example of the synthesis method of the organic compound represented by General Formula (G1-1a-1) is described below.

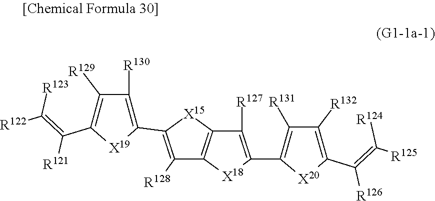

A variety of reactions can be applied to the synthesis method of the organic compound represented by General Formula (G1-1a-1). For example, synthesis reactions described below enable the synthesis of the organic compound represented by General Formula (G1-1a-1).

The organic compound of the present invention represented by General Formula (G1-1a-1) can be synthesized by Reaction Schemes (s2-1) to (s2-4) below.

First, Reaction Scheme (s2-1) is described. Ethynylation of a heteroaryl compound (Compound 17) can form an ethynyl-group-substituted heteroaryl compound (Compound 18). Reaction Scheme (s2-1) is shown below.

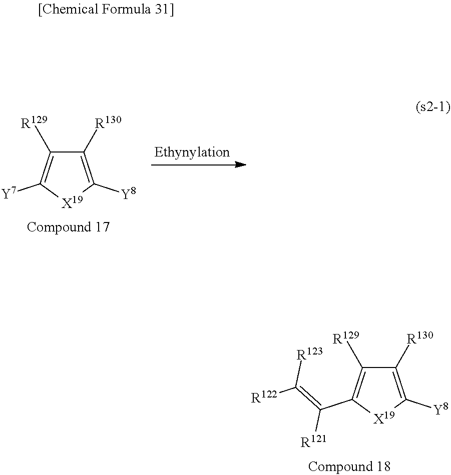

Next, Reaction Scheme (s2-2) is described. Ethynylation of a heteroaryl compound (Compound 19) can form an ethynyl-group-substituted heteroaryl compound (Compound 20). Reaction Scheme (s2-2) is shown below.

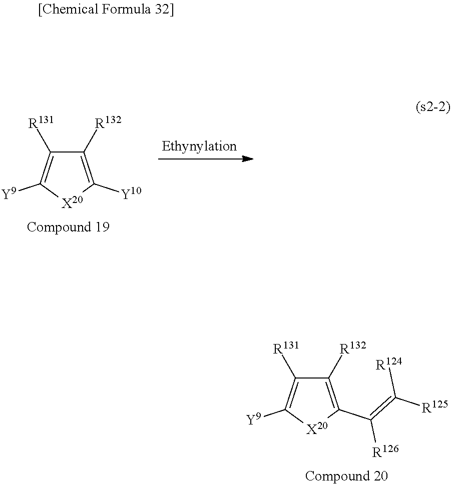

Next, Reaction Schemes (s2-3) and (s2-4) are described. Coupling between the heteroaryl compound (Compound 18) and a heteroaryl compound (Compound 21) can form a heteroaryl compound (Compound 22), and then coupling between the heteroaryl compound (Compound 22) and the heteroaryl compound (Compound 20) can form the organic compound represented by General Formula (G1-1a-1), which is the objective substance. Reaction Schemes (s2-3) and (s2-4) are shown below.

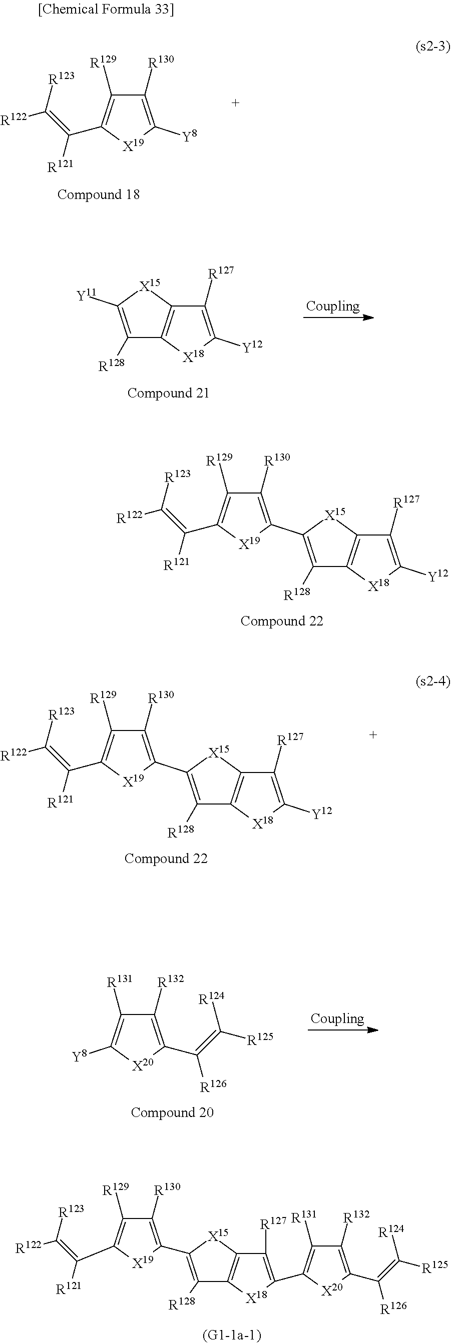

Note that in the case where the heteroaryl compound (Compound 18) and the heteroaryl compound (Compound 20) have the same structure, coupling between one equivalent of the heteroaryl compound (Compound 21) and two equivalents of the heteroaryl compound (Compound 18 or 20) can form the organic compound represented by General Formula (G1-1a-1) from the heteroaryl compound (Compound 21) in one step.

### Synthesis Method 2 of Organic Compound Represented by General Formula (G1-1a-1)

An example of the synthesis method of the organic compound represented by General Formula (G1-1a-1) is described below. The organic compound represented by General Formula (G1-1a-1), which is the objective substance, can also be obtained by Reaction Schemes (s2-5) to (s2-8).

First, Reaction Schemes (s2-5) and (s2-6) are described. Coupling between the heteroaryl compound (Compound 21) and a heteroaryl compound (Compound 17) can form a heteroaryl compound (Compound 23), and then coupling between the heteroaryl compound (Compound 23) and the heteroaryl compound (Compound 19) can form a heteroaryl compound (Compound 24). Reaction Schemes (s2-5) and (s2-6) are shown below.

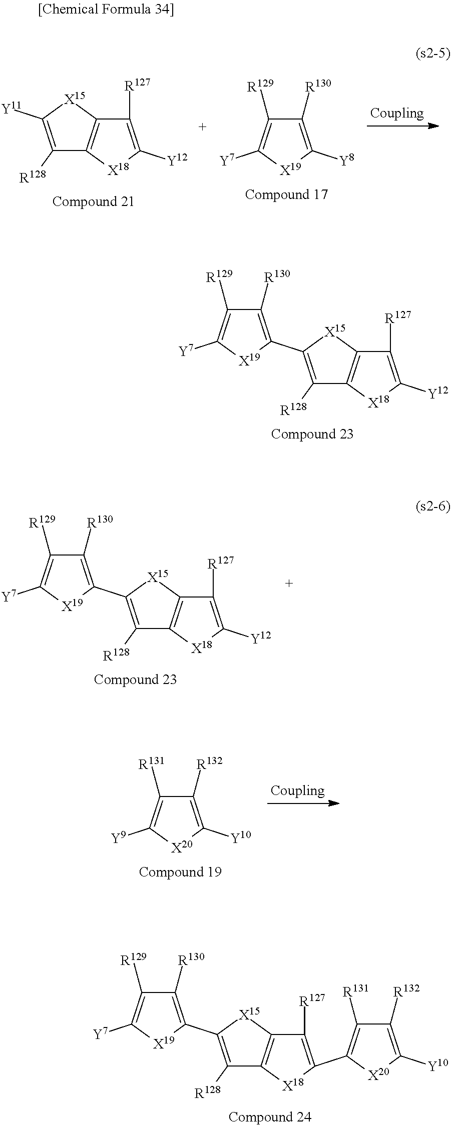

Note that in the case where the heteroaryl compound (Compound 17) and the heteroaryl compound (Compound 19) have the same structure, coupling between one equivalent of the heteroaryl compound (Compound 21) and two equivalents of the heteroaryl compound (Compound 17 or 19) can form the heteroaryl compound (Compound 24) from the heteroaryl compound (Compound 21) in one step.

Next, Reaction Schemes (s2-7) and (s2-8) are described. Ethynylation of the heteroaryl compound (Compound 24) can form an ethynyl-group-substituted heteroaryl compound (Compound 25), and then ethynylation of the heteroaryl compound (Compound 25) can form the organic compound represented by General Formula (G1-1a-1), which is the objective substance. Reaction Schemes (s2-7) and (s2-8) are shown below.

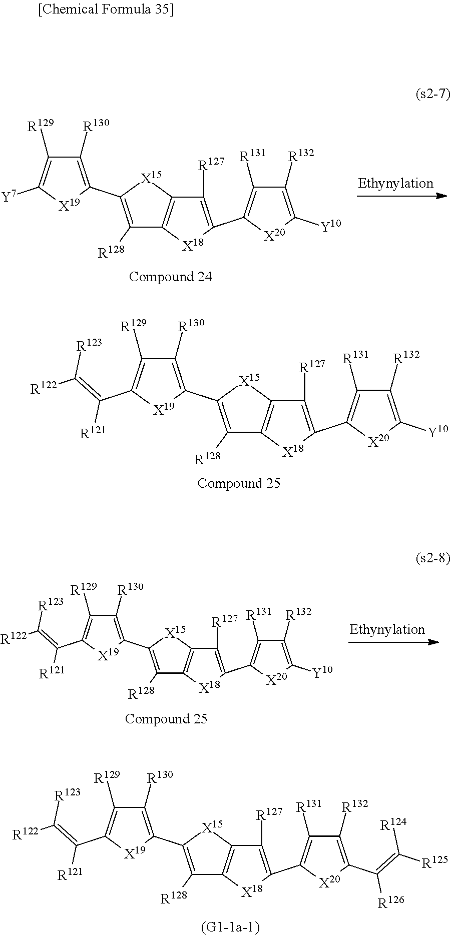

Note that in the case where the functional group Y⁷ and the functional group Y¹⁰ in the heteroaryl compound (Compound 24) are converted to the same substituents, ethynylation of one equivalent of the heteroaryl compound (Compound 24) is performed using two equivalents of reagent, whereby the organic compound represented by General Formula (G1-1a-1) can be obtained from the heteroaryl compound (Compound 24) in one step.

### Synthesis Method 1 of Organic Compound Represented by General Formula (G1-1a-2)

An example of the synthesis method of the organic compound represented by General Formula (G1-1a-2) is described below.

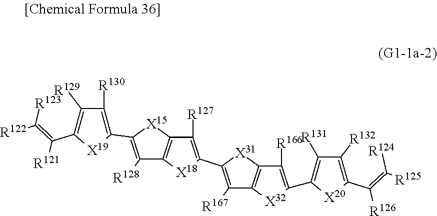

A variety of reactions can be applied to the synthesis method of the organic compound represented by General Formula (G1-1a-2). For example, synthesis reactions described below enable the synthesis of the organic compound represented by General Formula (G1-1a-2).

The organic compound of the present invention represented by General Formula (G1-1a-2) can be synthesized by Reaction Schemes (s3-1) and (s3-2) below.

Reaction Schemes (s3-1) and (s3-2) are described. Coupling between a heteroaryl compound (Compound 26) and the heteroaryl compound (Compound 18) can form a heteroaryl compound (Compound 27), and then coupling between the heteroaryl compound (Compound 27) and the heteroaryl compound (Compound 20) can form the organic compound represented by General Formula (G1-1a-2), which is the objective substance. Reaction Schemes (s3-1) and (s3-2) are shown below.

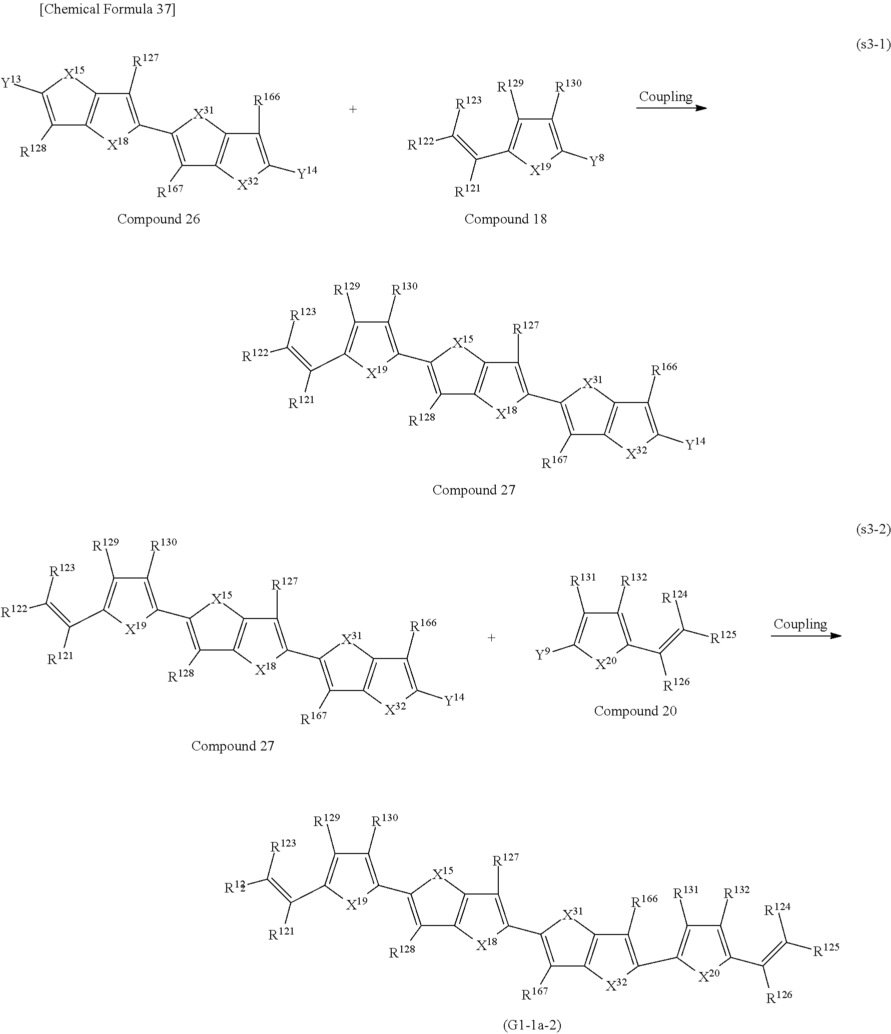

Note that in the case where the heteroaryl compound (Compound 18) and the heteroaryl compound (Compound 20) have the same structure, coupling between one equivalent of the heteroaryl compound (Compound 26) and two equivalents of the heteroaryl compound (Compound 18 or 20) can form the organic compound represented by General Formula (G1-1a-2), which is the objective substance, from the heteroaryl compound (Compound 26) in one step.

### Synthesis Method 2 of Organic Compound Represented by General Formula (G1-1a-2)

An example of the synthesis method of the organic compound represented by General Formula (G1-1a-2) is described below. The organic compound represented by General Formula (G1-1a-2), which is the objective substance, can also be obtained by Reaction Schemes (s3-3) and (s3-4).

First, Reaction Scheme (s3-3) is described. Coupling between a heteroaryl compound (Compound 28) and the heteroaryl compound (Compound 20) can form a heteroaryl compound (Compound 29). Reaction Scheme (s3-3) is shown below.

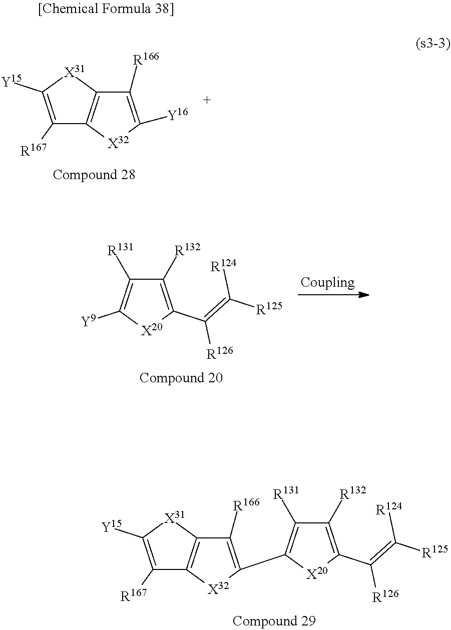

Next, Reaction Scheme (s3-4) is described. Coupling between the heteroaryl compound (Compound 22) obtained by Reaction Scheme (S2-3) and the heteroaryl compound (Compound 29) can form the organic compound represented by General Formula (G1-1a-2), which is the objective substance. Reaction Scheme (s3-4) is shown below.

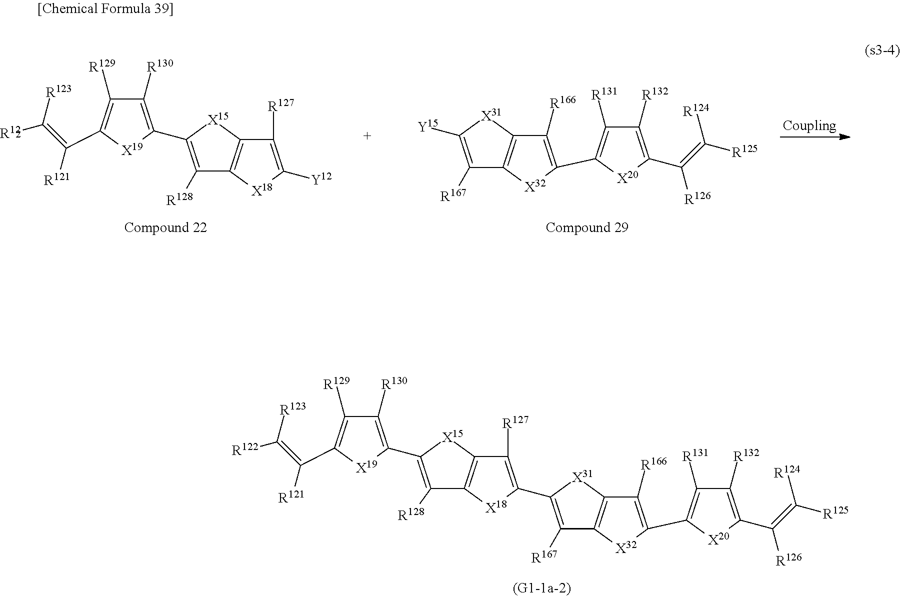

In Reaction Schemes (s1-1) to (s1-16), (s2-1) to (s2-8), and (s3-1) to (s3-4) above, Y¹ to Y⁶, Y⁸, Y⁹, and Y¹¹ to Y¹⁶ each independently represent hydrogen, a halogen, a boronic acid group, an organoboron group, a triflate group, an organotin group, an organozinc group, a magnesium halide group, or the like; and Y⁷ and Y¹⁰ each independently represent hydrogen, a halogen, a triflate group, a formyl group, or the like.

One of Y¹ and Y³ represents a boronic acid group, an organoboron group, an organotin group, an organozinc group, an amino group, a magnesium halide group, or the like; and the other of Y¹ and Y³ represents hydrogen, chlorine, bromine, iodine, a triflate group, or the like. The same applies to combinations of Y¹ and Y⁵, Y² and Y⁴, Y² and Y⁶, Y⁸ and Y¹¹, Y⁸ and Y¹³, Y⁹ and Y¹², Y⁹ and Y¹⁴, Y⁹ and Y¹⁶, and Y¹² and Y¹⁵. The halogen is preferably chlorine, bromine, or iodine; bromine or iodine is preferred in terms of reactivity, and chlorine or bromine is preferred in terms of cost.

In the case where a Migita-Kosugi-Stille coupling reaction using a palladium catalyst is performed in Reaction Schemes (s1-11) to (s1-14), (s2-3) to (s2-6), and (s3-1) to (s3-4), Y¹ to Y⁶, Y⁸, Y⁹, and Y¹¹ to Y¹⁶ represent a halogen, an organotin group, or a triflate group, and the halogen is preferably iodine, bromine, or chlorine. In the reaction, a palladium compound such as bis(dibenzylideneacetone)palladium(0), palladium(II) acetate, [1,1-bis(diphenylphosphino)ferrocene]palladium(II) dichloride, or tetrakis(triphenylphosphine)palladium(0) and a ligand such as tri(tert-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, or tri(ortho-tolyl)phosphine can be used. In the reaction, an organic base such as sodium tert-butoxide, an inorganic base such as potassium carbonate, cesium carbonate, or sodium carbonate, or the like can be used.

In the reaction, toluene, xylene, benzene, tetrahydrofuran, dioxane, ethanol, methanol, water, diethylene glycol dimethyl ether, ethylene glycol monomethyl ether, or the like can be used as a solvent. Reagents that can be used for the reaction are not limited thereto.

The reactions represented by Reaction Schemes (s1-11) to (s1-14), (s2-3) to (s2-6), and (s3-1) to (s3-4) can be performed using a Suzuki-Miyaura coupling reaction using an organoboron compound, a Kumada-Tamao-Corriu coupling reaction using a Grignard reagent, a Negishi coupling reaction using an organozinc compound, a reaction using copper or a copper compound, or the like.

The reactions represented by Reaction Schemes (s1-1) to (s1-10), (s1-15) to (s1-18), (s2-1), (s2-2), (s2-7), and (s2-8) can be performed using a Knoevenagel condensation reaction, a chlorination reaction, a bromination reaction, an iodination reaction, or the like.

In the Knoevenagel condensation reaction, malononitrile or the like can be used as a reaction reagent.

In the Knoevenagel condensation reaction, toluene, xylene, acetic acid, ethyl acetate, methanol, ethanol, isopropanol, water, or the like can be used as a solvent.

In the Knoevenagel condensation reaction, piperidine, pyridine, triethylamine, proline, or the like can be used as a base catalyst.

In the chlorination reaction, N-chlorosuccinimide, oxalyl chloride, or the like can be used as a reaction reagent.

In the bromination reaction, N-bromosuccinimide, N-bromophthalimide, bromine, or the like can be used as a reaction reagent.

In the iodination reaction, N-iodosuccinimide, N-iodophthalimide, iodine, or the like can be used as a reaction reagent.

In the halogenation reaction, chloroform, dichloroethane, dichloromethane, N,N-dimethylformamide, toluene, xylene, N-methyl-2-pyrrolidone, acetonitrile, acetic acid, ethyl acetate, or the like can be used as a solvent.

In the halogenation reaction, an inorganic base such as sodium hydrogen carbonate, potassium carbonate, cesium carbonate, or sodium carbonate, or the like can be used.

The halogen substituted for a compound by the halogenation reaction can be converted into a boronic acid group, an organoboron group, an organotin group, an organozinc group, an amino group, a magnesium halide group, a triflate group, a formyl group, or the like. In other words, a halogen-substituted compound by the halogenation reaction can be used in a Suzuki-Miyaura coupling reaction using an organoboron compound, a Migita-Kosugi-Stille coupling reaction using an organotin compound, a Kumada-Tamao-Corriu coupling reaction using a Grignard reagent, a Negishi coupling reaction using an organozinc compound, a reaction using copper or a copper compound, or the like.

The methods for synthesizing the organic compounds of embodiments of the present invention represented by General Formulas (G1), (G1-1a-1), and (G1-1a-2) are not limited to Reaction Schemes (s1-1) to (s1-16), (s2-1) to (s2-8), and (s3-1) to (s3-4).

This embodiment can be combined with any of the other embodiments in this specification as appropriate.

### Embodiment 2

In this embodiment, a light-receiving device of one embodiment of the present invention will be described.

The light-receiving device of one embodiment of the present invention has a function of sensing light (hereinafter, also referred to as a light-receiving function).

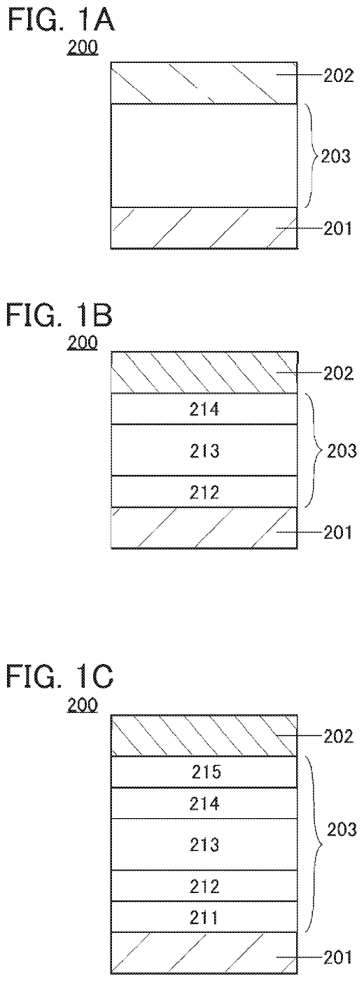

FIGS. 1A to 1C are each a schematic cross-sectional view of a light-receiving device 200 of one embodiment of the present invention.

### Basic Structure of Light-Receiving Device

Basic structures of the light-receiving device will be described. FIG. 1A illustrates a light-receiving device 200 including at least a light-receiving layer 203 including an active layer and a carrier-transport layer between a pair of electrodes. Specifically, an EL layer 203 is interposed between the first electrode 201 and the second electrode 202.

FIG. 1B illustrates a stacked-layer structure of the light-receiving layer 203 in the light-receiving device 200 of one embodiment of the present invention. The light-receiving layer 203 has a structure in which a first carrier-transport layer 212, an active layer 213, and a second carrier-transport layer 214 are sequentially stacked over the first electrode 201.

FIG. 1C illustrates a stacked-layer structure of the light-receiving layer 203 in the light-receiving device 200 of one embodiment of the present invention. The light-receiving layer 203 has a structure in which a first carrier-injection layer 211, the first carrier-transport layer 212, the active layer 213, the second carrier-transport layer 214, and a second carrier-injection layer 215 are sequentially stacked over the first electrode 201.

### Specific Structure of Light-Receiving Device

Next, a specific structure of the light-receiving device 200 of one embodiment of the present invention will be described. Here, description is made with reference to FIG. 1C.

### First Electrode and Second Electrode

The first electrode 201 and the second electrode 202 can be formed using materials that can be used for a first electrode 101 and a second electrode 102, which will be described in Embodiment 3.

Note that a microcavity structure can be obtained when the first electrode 201 is a reflective electrode and the second electrode 202 is a semi-transmissive and semi-reflective electrode, for example. The microcavity structure can intensify light with a specific wavelength to be sensed, thereby achieving a light-receiving device with high sensitivity.

### First Carrier-Injection Layer

The first carrier-injection layer 211 injects holes from the light-receiving layer 203 to the first electrode 201, and contains a material with a high hole-injection property. Examples of the material with a high hole-injection property include an aromatic amine compound and a composite material containing a hole-transport material and an acceptor material (electron-accepting material).

The first carrier-injection layer 211 can be formed using a material that can be used for a hole-injection layer 111, which will be described in Embodiment 3.

### First Carrier-Transport Layer

The first carrier-transport layer 212 transports holes generated in the active layer 213 on the basis of incident light to the first electrode 201, and contains a hole-transport material (also referred to as a first organic compound). The hole-transport material preferably has a hole mobility of 1×10⁻⁶ cm²/Vs or higher. Note that other substances can also be used as long as the substances have a hole-transport property higher than an electron-transport property.

As the hole-transport material (first organic compound), a π-electron rich heteroaromatic compound or an aromatic amine (a compound having an aromatic amine skeleton) can be used.

Alternatively, a carbazole derivative, a thiophene derivative, or a furan derivative can be used as the hole-transport material (first organic compound).

As the hole-transport material (first organic compound), an aromatic monoamine compound or a heteroaromatic monoamine compound having at least one skeleton of biphenylamine, carbazolylamine, dibenzofuranylamine, dibenzothiophenylamine, fluorenylamine, and spirofluorenylamine can be used.

Alternatively, as the hole-transport material (first organic compound), an aromatic monoamine compound or a heteroaromatic monoamine compound having two or more skeletons selected from biphenylamine, carbazolylamine, dibenzofuranylamine, dibenzothiophenylamine, fluorenylamine, and spirofluorenylamine can be used.

In the case where the hole-transport material (first organic compound) is an aromatic monoamine compound or a heteroaromatic monoamine compound having two or more skeletons selected from biphenylamine, carbazolylamine, dibenzofuranylamine, dibenzothiophenylamine, fluorenylamine, and spirofluorenylamine, one nitrogen atom may be shared by two or more skeletons. For example, in the case where fluorene and biphenyl are bonded to a nitrogen atom of a monoamine in an aromatic monoamine compound, the compound can be regarded as an aromatic monoamine compound having a fluorenylamine skeleton and a biphenylamine skeleton.

Note that each of biphenylamine, carbazolylamine, dibenzofuranylamine, dibenzothiophenylamine, fluorenylamine, and spirofluorenylamine listed above as the skeleton included in the hole-transport material (first organic compound) may include a substituent. Examples of the substituent include a substituted or unsubstituted aryl group having 6 to 30 carbon atoms, a substituted or unsubstituted alkyl group having 1 to 20 carbon atoms, a substituted or unsubstituted cycloalkyl group having 1 to 20 carbon atoms, and a substituted or unsubstituted heteroaryl group having 4 to 30 carbon atoms.

The hole-transport material (first organic compound) is preferably an amine compound having a triarylamine skeleton (a heteroaryl group or a carbazolyl group is also included as an aryl group in a triarylamine compound).

The first carrier-transport layer 212 can also be formed using a material that can be used for a hole-transport layer 112, which will be described in Embodiment 3.

The first carrier-transport layer 212 is not limited to a single layer, and may be a stack of two or more layers each containing any of the above substances; each of the layers may be a mixed layer containing two or more kinds of compounds.

In the light-receiving device described in this embodiment, the active layer 213 can be formed using the same organic compound as the first carrier-transport layer 212. The use of the same organic compound for the first carrier-transport layer 212 and the active layer 213 is preferable, in which case carriers can be efficiently transported from the first carrier-transport layer 212 to the active layer 213.

### Active Layer

The active layer 213 generates carriers on the basis of incident light and contains a semiconductor. Examples of the semiconductor include an inorganic semiconductor such as silicon and an organic semiconductor including an organic compound. This embodiment shows an example in which an organic semiconductor is used as the semiconductor contained in the active layer. The use of an organic semiconductor is preferable because the light-emitting layer and the active layer provided in the same device can be formed by the same method (e.g., a coating method or a vacuum evaporation method) and thus the same manufacturing apparatus can be used.

The active layer 213 contains at least a third organic compound and a fourth organic compound.

Examples of the third organic compound include π-electron rich heteroaromatic ring compounds or electron-donating compounds, such as copper(II) phthalocyanine (CuPc), tetraphenyldibenzoperiflanthene (DBP), zinc phthalocyanine (ZnPc), tin phthalocyanine (SnPc), and quinacridone.

Other examples of the third organic compound include a carbazole compound, a thiophene compound, a furan compound, and a compound having an aromatic amine skeleton. Other examples of the third organic compound include a naphthalene compound, an anthracene compound, a pyrene compound, a triphenylene compound, a fluorene compound, a pyrrole compound, a benzofuran compound, a benzothiophene compound, an indole compound, a dibenzofuran compound, a dibenzothiophene compound, an indolocarbazole compound, a porphyrin compound, a phthalocyanine compound, a naphthalocyanine compound, a quinacridone compound, a polyphenylene vinylene compound, a polyparaphenylene compound, a polyfluorene compound, a polyvinylcarbazole compound, and a polythiophene compound.

Examples of the fourth organic compound include π-electron deficient heteroaromatic ring compounds or electron-accepting compounds, such as a perylenetetracarboxylic diimide (PTCDI) compound, an oxadiazole compound, a triazole compound, an imidazole compound, an oxazole compound, a thiazole compound, a phenanthroline compound, a quinoline compound, a benzoquinoline compound, a quinoxaline compound, a dibenzoquinoxaline compound, a pyridine compound, a bipyridine compound, a pyrimidine compound, a naphthalene compound, an anthracene compound, a coumalin compound, a rhodamine compound, a triazine compound, a quinone compound, a metal complex having a quinoline skeleton, a metal complex having a benzoquinoline skeleton, a metal complex having an oxazole skeleton, and a metal complex having a thiazole skeleton.

Examples of the fourth organic compound include electron-accepting organic semiconductor materials such as fullerene (e.g., C₆₀ and C₇₀) and fullerene compounds. Fullerene has a soccer ball-like shape, which is energetically stable. Both the HOMO level and the LUMO level of fullerene are deep (low). Having a deep LUMO level, fullerene has an extremely high electron-accepting property (acceptor property). When π-electron conjugation (resonance) spreads on a plane as in benzene, an electron-donating property (donor property) usually increases; however, fullerene has a spherical shape, and thus has a high electron-accepting property although π-electron conjugation widely spread therein. The high electron-accepting property efficiently causes rapid charge separation and thus is useful for light-receiving devices. Both C₆₀ and C₇₀ have a wide absorption band in the visible light region, and C₇₀ is especially preferable because of having a larger π-electron conjugation system and a wider absorption band in the long wavelength region than C₆₀. Other examples of fullerene compounds include [6,6]-phenyl-C₇₁-butyric acid methyl ester (abbreviation: PC₇₀BM), [6,6]-phenyl-C₆₁-butyric acid methyl ester (abbreviation: PC₆₀BM), and 1′,1″,4′,4″-tetrahydro-di[1,4]methanonaphthaleno[1,2:2′,3′,56,60:2″,3″ ][5,6]fullerene-C₆₀ (abbreviation: ICBA).

The active layer 213 is preferably a stacked film of a first layer containing the third organic compound and a second layer containing the fourth organic compound.

In the light-receiving device having any of the aforementioned structures, the active layer 213 is preferably a mixed film containing the third organic compound and the fourth organic compound.

The HOMO level of the electron-donating organic semiconductor material is preferably shallower (higher) than the HOMO level of the electron-accepting organic semiconductor material. The LUMO level of the electron-donating organic semiconductor material is preferably shallower (higher) than the LUMO level of the electron-accepting organic semiconductor material.

Fullerene having a spherical shape may be used as the electron-accepting organic semiconductor material, and an organic semiconductor material having a substantially planar shape may be used as the electron-donating organic semiconductor material. Molecules of similar shapes tend to aggregate, and aggregated molecules of similar kinds, which have molecular orbital energy levels close to each other, can increase the carrier-transport property.

### Second Carrier-Transport Layer

The second carrier-transport layer 214 transports electrons generated in the active layer 213 on the basis of incident light to the second electrode 202, and contains an electron-transport material (also referred to as a second organic compound). The electron-transport material preferably has an electron mobility of 1×10⁶ cm²/Vs or higher. Note that other substances can also be used as long as the substances have an electron-transport property higher than a hole-transport property.

As the electron-transport material (second organic compound), a π-electron deficient heteroaromatic compound can be used.

As the electron-transport material (second organic compound), any of the following materials can be used, for example: a metal complex having a quinoline skeleton, a metal complex having a benzoquinoline skeleton, a metal complex having an oxazole skeleton, a metal complex having a thiazole skeleton, an oxadiazole derivative, a triazole derivative, an imidazole derivative, an oxazole derivative, a thiazole derivative, a phenanthroline derivative, a quinoline derivative having a quinoline ligand, a benzoquinoline derivative, a quinoxaline derivative, a dibenzoquinoxaline derivative, a pyridine derivative, a bipyridine derivative, a pyrimidine derivative, and a π-electron deficient heteroaromatic compound such as a nitrogen-containing heteroaromatic compound.

Alternatively, the electron-transport material (second organic compound) is a compound having a triazine ring.

The second carrier-transport layer 214 can be formed using a material that can be used for an electron-transport layer 114, which will be described in Embodiment 3.

The second carrier-transport layer 214 is not limited to a single layer and may be a stack of two or more layers each containing any of the above substances.

### Second Carrier-Injection Layer

The second carrier-injection layer 215 is a layer for increasing the efficiency of electron injection from the light-receiving layer 203 to the second electrode 202, and contains a material with a high electron-injection property. As the material with a high electron-injection property, an alkali metal, an alkaline earth metal, or a compound thereof can be used. As the material with a high electron-injection property, a composite material containing an electron-transport material and a donor material (electron-donating material) can also be used.

The second carrier-injection layer 215 can be formed using a material that can be used for an electron-injection layer 115, which will be described in Embodiment 3.

A structure in which a plurality of light-receiving layers are stacked between a pair of electrodes (the structure is also referred to as a tandem structure) can be obtained by providing a charge-generation layer between two light-receiving layers 203. In addition, three or more light-receiving layers may be stacked with charge-generation layers each provided between adjacent light-receiving layers. The charge-generation layer can be formed using a material that can be used for a charge-generation layer 106, which will be described in Embodiment 3.

Materials that can be used for the layers (the first carrier-injection layer 211, the first carrier-transport layer 212, the active layer 213, the second carrier-transport layer 214, and the second carrier-injection layer 215) included in the light-receiving layer 203 of the light-receiving device described in this embodiment are not limited to the materials described in this embodiment, and other materials can be used in combination as long as the functions of the layers are fulfilled.

Note that in this specification and the like, the terms “layer” and “film” can be interchanged with each other as appropriate.

Note that the light-receiving device of one embodiment of the present invention has a function of sensing visible light. The light-receiving device of one embodiment of the present invention has sensitivity to visible light. The light-receiving device of one embodiment of the present invention further preferably has a function of sensing visible light and infrared light. The light-receiving device of one embodiment of the present invention preferably has sensitivity to visible light and infrared light.

In this specification and the like, a blue (B) wavelength range is greater than or equal to 400 nm and less than 490 nm, and blue (B) light has at least one emission spectrum peak in the wavelength range. A green (G) wavelength range is greater than or equal to 490 nm and less than 580 nm, and green (G) light has at least one emission spectrum peak in the wavelength range. A red (R) wavelength range is greater than or equal to 580 nm and less than 700 nm, and red (R) light has at least one emission spectrum peak in the wavelength range. In this specification and the like, a visible light wavelength range is greater than or equal to 400 nm and less than 700 nm, and visible light has at least one emission spectrum peak in the wavelength range. An infrared (IR) wavelength range is greater than or equal to 700 nm and less than 900 nm, and infrared (IR) light has at least one emission spectrum peak in the wavelength range.

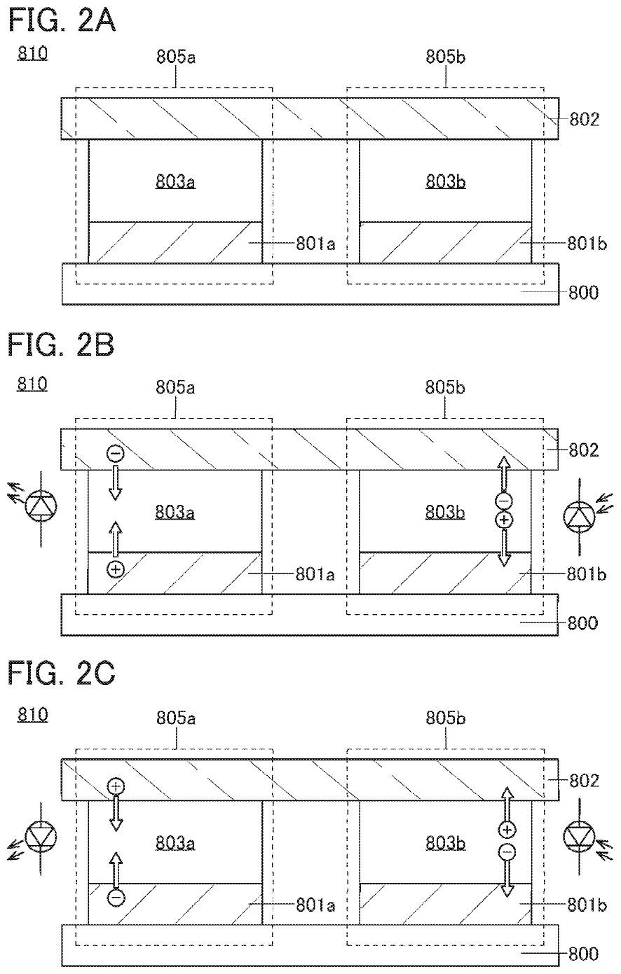

The above-described light-receiving device of one embodiment of the present invention can be used for a display apparatus including an organic EL device. In other words, the light-receiving device of one embodiment of the present invention can be incorporated into a display apparatus including an organic EL device. As an example, FIG. 2A illustrates a schematic cross-sectional view of a light-emitting and light-receiving apparatus 810 used as a display apparatus in which a light-emitting device 805a and a light-receiving device 805b are formed over the same substrate.

The light-emitting and light-receiving apparatus 810 includes the light-emitting device 805a and the light-receiving device 805b, and thus has one or both of an imaging function and a sensing function in addition to a function of displaying an image.

The light-emitting device 805a has a function of emitting light (hereinafter, also referred to as a light-emitting function). The light-emitting device 805a includes an electrode 801a, an EL layer 803a, and an electrode 802. Thus, the EL layer 803a interposed between the electrode 801a and the electrode 802 at least includes a light-emitting layer. The light-emitting layer contains a light-emitting substance. The EL layer 803a emits light when a voltage is applied between the electrode 801a and the electrode 802. The EL layer 803a may include any of a variety of layers such as a hole-injection layer, a hole-transport layer, an electron-transport layer, an electron-injection layer, a carrier-blocking (hole-blocking or electron-blocking) layer, and a charge-generation layer, in addition to the light-emitting layer. For the light-emitting device 805a, a structure of the light-emitting device, which is an organic EL device to be described in Embodiment 3, can be employed.

The light-receiving device 805b has a function of sensing light (hereinafter, also referred to as a light-receiving function). The light-emitting device 805b includes an electrode 801b, a light-receiving layer 803b, and the electrode 802. The light-receiving layer 803b interposed between the electrode 801b and the electrode 802 at least includes an active layer. The light-receiving device 805b functions as a photoelectric conversion device; when light is incident on the light-receiving layer 803b, electric charge can be generated and extracted as a current. At this time, a voltage may be applied between the electrode 801b and the electrode 802. The amount of generated electric charge depends on the amount of the light incident on the light-receiving layer 803b. For the light-receiving device 805b, the structure of the above-described light-receiving device 200 can be employed.

The light-receiving device 805b, which is easily made thin, lightweight, and large in area and has a high degree of freedom for shape and design, can be used in a variety of display apparatuses. In addition, the EL layer 803a included in the light-emitting device 805a and the light-receiving layer 803b included in the light-receiving device 805b can be formed by the same method (e.g., a vacuum evaporation method) with the same manufacturing apparatus, which is preferable.

The electrode 801a and the electrode 801b are provided on the same plane. In FIG. 2A, the electrodes 801a and 801b are provided over a substrate 800. The electrodes 801a and 801b can be formed by processing a conductive film formed over the substrate 800 into an island shape, for example. In other words, the electrodes 801a and 801b can be formed through the same process.

As the substrate 800, a substrate having heat resistance high enough to withstand the formation of the light-emitting device 805a and the light-receiving device 805b can be used. When an insulating substrate is used as the substrate 800, a glass substrate, a quartz substrate, a sapphire substrate, a ceramic substrate, an organic resin substrate, or the like can be used. Alternatively, a semiconductor substrate can be used. For example, a single crystal semiconductor substrate or a polycrystalline semiconductor substrate of silicon, silicon carbide, or the like; a compound semiconductor substrate of silicon germanium or the like; an SOI substrate; or the like can be used.

As the substrate 800, it is particularly preferable to use the insulating substrate or the semiconductor substrate over which a semiconductor circuit including a semiconductor device such as a transistor is formed. The semiconductor circuit preferably forms a pixel circuit, a gate line driver circuit (a gate driver), a source line driver circuit (a source driver), or the like. In addition to the above, an arithmetic circuit, a memory circuit, or the like may be formed.

The electrode 802 is formed of a layer shared by the light-emitting device 805a and the light-receiving device 805b. As the electrode through which light enters or exits, a conductive film that transmits visible light and infrared light is used. As the electrode through which light neither enters nor exits, a conductive film that reflects visible light and infrared light is preferably used.

The electrode 802 in the display device of one embodiment of the present invention functions as one of the electrodes in each of the light-emitting device 805a and the light-receiving device 805b.

In FIG. 2B, the electrode 801a of the light-emitting device 805a has a potential higher than that of the electrode 802. In this case, the electrode 801a functions as an anode and the electrode 802 functions as a cathode in the light-emitting device 805a. The electrode 801b of the light-receiving device 805b has a potential lower than that of the electrode 802. For easy understanding of the direction of current flow, FIG. 2B illustrates a circuit symbol of a light-emitting diode on the left of the light-emitting device 805a and a circuit symbol of a photodiode on the right of the light-receiving device 805b. The flow directions of carriers (electrons and holes) in each device are also schematically indicated by arrows.

In the structure illustrated in FIG. 2B, when a first potential is supplied to the electrode 801a through a first wiring, a second potential is supplied to the electrode 802 through a second wiring, and a third potential is supplied to the electrode 801b through a third wiring, the following relationship is satisfied: the first potential>the second potential>the third potential.

In FIG. 2C, the electrode 801a of the light-emitting device 805a has a potential lower than that of the electrode 802. In this case, the electrode 801a functions as a cathode and the electrode 802 functions as an anode in the light-emitting device 805a. The electrode 801b of the light-receiving device 805b has a potential lower than that of the electrode 802 and a potential higher than that of the electrode 801a. For easy understanding of the direction of current flow, FIG. 2C illustrates a circuit symbol of a light-emitting diode on the left of the light-emitting device 805a and a circuit symbol of a photodiode on the right of the light-receiving device 805b. The flow directions of carriers (electrons and holes) in each device are also schematically indicated by arrows.

In the structure illustrated in FIG. 2C, when a first potential is supplied to the electrode 801a through a first wiring, a second potential is supplied to the electrode 802 through a second wiring, and a third potential is supplied to the electrode 801b through a third wiring, the following relationship is satisfied: the second potential>the third potential>the first potential.

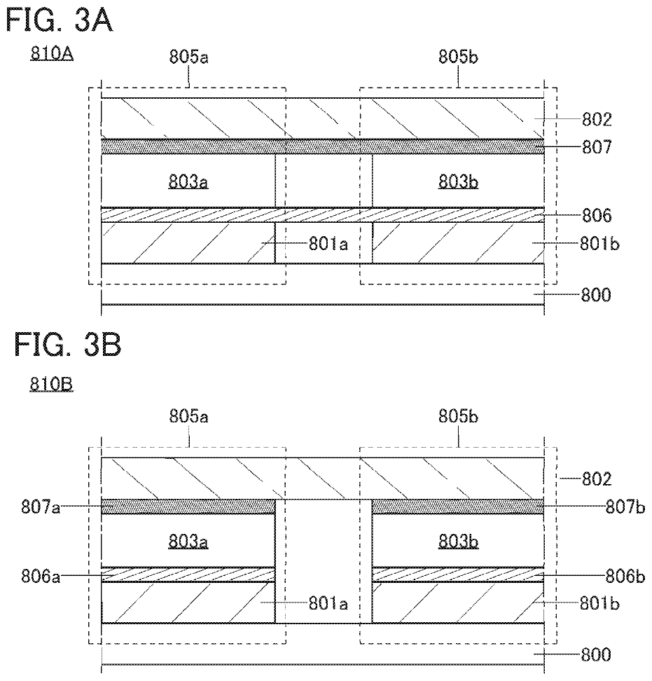

FIG. 3A illustrates a light-emitting and light-receiving apparatus 810A that is a variation example of the light-emitting and light-receiving apparatus 810. The light-emitting and light-receiving apparatus 810A is different from the light-emitting and light-receiving apparatus 810 in including a common layer 806 and a common layer 807. In the light-emitting device 805a, the common layers 806 and 807 function as part of the EL layer 803a. The common layer 806 includes a hole-injection layer and a hole-transport layer, for example. The common layer 807 includes an electron-transport layer and an electron-injection layer, for example.

With the common layers 806 and 807, a light-receiving device can be incorporated without a significant increase in the number of times of separate coloring, whereby the light-emitting and light-receiving apparatus 810A can be manufactured with a high throughput.

FIG. 3B illustrates a light-emitting and light-receiving apparatus 810B that is a variation example of the light-emitting and light-receiving apparatus 810. The light-emitting and light-receiving apparatus 810B is different from the light-emitting and light-receiving apparatus 810A in that the EL layer 803a includes a layer 806a and a layer 807a and the light-receiving layer 803b includes a layer 806b and a layer 807b. The layers 806a and 806b are formed using different materials, and each include a hole-injection layer and a hole-transport layer, for example. Note that the layers 806a and 806b may be formed using the same material. The layers 807a and 807b are formed using different materials, and each include an electron-transport layer and an electron-injection layer, for example. Note that the layers 807a and 807b may be formed using the same material.

An optimum material for forming the light-emitting device 805a is selected for the layers 806a and 807a and an optimum material for forming the light-receiving device 805b is selected for the layers 806b and 807b, whereby the light-emitting device 805a and the light-receiving device 805b can have higher performance in the light-emitting and light-receiving apparatus 810B.

The pixel resolution of the light-receiving device 805b can be 100 ppi or more, preferably 200 ppi or more, further preferably 300 ppi or more, still further preferably 400 ppi or more, and yet further preferably 500 ppi or more, and 2000 ppi or less, 1000 ppi or less, or 600 ppi or less, for example. In particular, when the resolution of the light-receiving device 805b is 200 ppi or more and 600 ppi or less, preferably 300 ppi or more and 600 ppi or less, the light-emitting and light-receiving apparatus of one embodiment of the present invention can be suitably used for image capturing of a fingerprint. In fingerprint authentication with the light-emitting and light-receiving apparatus 810, the increased resolution of the light-receiving device 805b enables, for example, highly accurate extraction of the minutiae of fingerprints; thus, the accuracy of the fingerprint authentication can be increased. The resolution is preferably 500 ppi or more, in which case the authentication conforms to the standard by the National Institute of Standards and Technology (NIST) or the like. On the assumption that the resolution of the light-receiving device is 500 ppi, the size of each pixel is 50.8 μm, which is adequate for image capturing of a fingerprint ridge distance (typically, greater than or equal to 300 μm and less than or equal to 500 μm).

The structures described in this embodiment can be used in appropriate combination with any of the structures described in the other embodiments.

### Embodiment 3

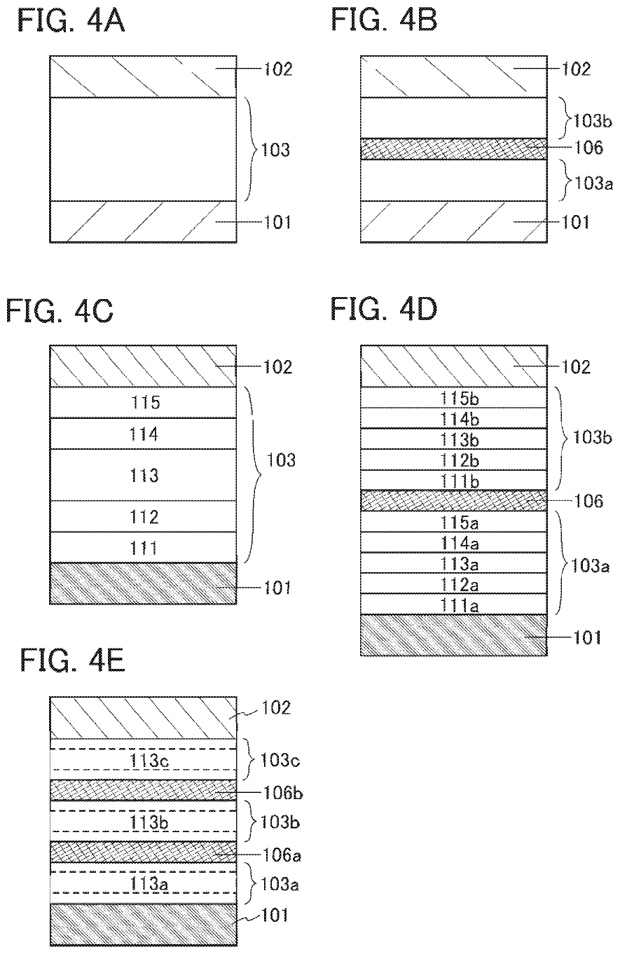

In this embodiment, other structures of the light-emitting devices described in Embodiment 2 will be described with reference to FIGS. 4A to 4E.

### Basic Structure of Light-Emitting Device

Basic structures of the light-emitting device are described. FIG. 4A illustrates a light-emitting device including, between a pair of electrodes, an EL layer including a light-emitting layer. Specifically, an EL layer 103 is interposed between a first electrode 101 and a second electrode 102.

FIG. 4B illustrates a light-emitting device that has a stacked-layer structure (tandem structure) in which a plurality of EL layers (two EL layers 103a and 103b in FIG. 4B) are provided between a pair of electrodes and the charge-generation layer 106 is provided between the EL layers. A light-emitting device having a tandem structure enables fabrication of a light-emitting apparatus that can be driven at a low voltage and has low power consumption.

The charge-generation layer 106 has a function of injecting electrons into one of the EL layers 103a and 103b and injecting holes into the other of the EL layers 103a and 103b when a potential difference is caused between the first electrode 101 and the second electrode 102. Thus, when a voltage is applied in FIG. 4B such that the potential of the first electrode 101 is higher than that of the second electrode 102, the charge-generation layer 106 injects electrons into the EL layer 103a and injects holes into the EL layer 103b.

Note that in terms of light extraction efficiency, the charge-generation layer 106 preferably has a property of transmitting visible light (specifically, the charge-generation layer 106 preferably has a visible light transmittance of 40% or more). The charge-generation layer 106 functions even if it has lower conductivity than the first electrode 101 or the second electrode 102.

FIG. 4C illustrates a stacked-layer structure of the EL layer 103 in the light-emitting device of one embodiment of the present invention. In this case, the first electrode 101 is regarded as functioning as an anode and the second electrode 102 is regarded as functioning as a cathode. The EL layer 103 has a structure in which the hole-injection layer 111, the hole-transport layer 112, the light-emitting layer 113, the electron-transport layer 114, and the electron-injection layer 115 are stacked in this order over the first electrode 101. Note that the light-emitting layer 113 may have a stacked-layer structure of a plurality of light-emitting layers that emit light of different colors. For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. The structure in which a plurality of light-emitting layers that emit light of the same color are stacked can sometimes achieve higher reliability than a single-layer structure. In the case where a plurality of EL layers are provided as in the tandem structure illustrated in FIG. 4B, the layers in each EL layer are sequentially stacked from the anode side as described above. When the first electrode 101 is the cathode and the second electrode 102 is the anode, the stacking order of the layers in the EL layer 103 is reversed. Specifically, the layer 111 over the first electrode 101 serving as the cathode is an electron-injection layer; the layer 112 is an electron-transport layer; the layer 113 is a light-emitting layer; the layer 114 is a hole-transport layer; and the layer 115 is a hole-injection layer.

The light-emitting layer 113 included in the EL layers (103, 103a, and 103b) contains an appropriate combination of a light-emitting substance and a plurality of substances, so that fluorescent or phosphorescent light of a desired emission color can be obtained. The light-emitting layer 113 may have a stacked-layer structure having different emission colors. In that case, one or both of light-emitting substances and other substances are different between the stacked light-emitting layers. Alternatively, the plurality of EL layers (103a and 103b) in FIG. 4B may exhibit their respective emission colors. Also in that case, one or both of the light-emitting substances and other substances are different between the stacked light-emitting layers.

The light-emitting device of one embodiment of the present invention can have a micro optical resonator (microcavity) structure when, for example, the first electrode 101 is a reflective electrode and the second electrode 102 is a semi-transmissive and semi-reflective electrode in FIG. 4C. Thus, light from the light-emitting layer 113 in the EL layer 103 can be resonated between the electrodes and light obtained through the second electrode 102 can be intensified.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is λ, the optical path length between the first electrode 101 and the second electrode 102 (the product of the thickness and the refractive index) is preferably adjusted to be mλ/2 (m is a natural number) or close to mλ/2.

To amplify desired light (wavelength: k) obtained from the light-emitting layer 113, it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (m′ is a natural number) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; thus, it is assumed that the above effect can be sufficiently obtained wherever the reflective region and the light-emitting region may be set in the first electrode 101 and the light-emitting layer that emits the desired light, respectively.

The light-emitting device illustrated in FIG. 4D is a light-emitting device having a tandem structure. Owing to a microcavity structure of the light-emitting device, light (monochromatic light) with different wavelengths from the EL layers (103a and 103b) can be extracted. Thus, separate coloring for obtaining a plurality of emission colors (e.g., R, G, and B) is not necessary. Therefore, high resolution can be easily achieved. A combination with coloring layers (color filters) is also possible. Furthermore, the emission intensity of light with a specific wavelength in the front direction can be increased, whereby power consumption can be reduced.

The light-emitting device illustrated in FIG. 4E is an example of the light-emitting device having the tandem structure illustrated in FIG. 4B, and includes three EL layers (103a, 103b, and 103c) stacked with charge-generation layers (106a and 106b) interposed therebetween, as illustrated in FIG. 4E. The three EL layers (103a, 103b, and 103c) include respective light-emitting layers (113a, 113b, and 113c), and the emission colors of the light-emitting layers can be selected freely. For example, each of the light-emitting layer 113a and the light-emitting layer 113c can emit blue light, and the light-emitting layer 113b can emit red light, green light, or yellow light. For another example, the light-emitting layer 113a can emit red light, the light-emitting layer 113b can emit blue light, green light, or yellow light, and the light-emitting layer 113c can emit red light.

In the light-emitting device of one embodiment of the present invention, at least one of the first electrode 101 and the second electrode 102 is a light-transmitting electrode (e.g., a transparent electrode or a semi-transmissive and semi-reflective electrode). In the case where the light-transmitting electrode is a transparent electrode, the transparent electrode has a visible light transmittance higher than or equal to 40%. In the case where the light-transmitting electrode is a semi-transmissive and semi-reflective electrode, the semi-transmissive and semi-reflective electrode has a visible light reflectance higher than or equal to 20% and lower than or equal to 80%, preferably higher than or equal to 40% and lower than or equal to 70%. These electrodes preferably have a resistivity of 1×10⁻² Ωcm or lower.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity of 1×10⁻² Ωcm or lower.

### Specific Structure of Light-Emitting Device

Next, a specific structure of the light-emitting device of one embodiment of the present invention will be described. Here, the description is made using FIG. 4D illustrating the tandem structure. Note that the structure of the EL layer applies also to the structure of the light-emitting devices having a single structure in FIGS. 4A and 4C. When the light-emitting device in FIG. 4D has a microcavity structure, the first electrode 101 is formed as a reflective electrode and the second electrode 102 is formed as a semi-transmissive and semi-reflective electrode. Thus, a single-layer structure or a stacked-layer structure can be formed using one or more kinds of desired electrode materials. Note that the second electrode 102 is formed after formation of the EL layer 103b, with the use of a material selected as described above.

### First Electrode and Second Electrode

As materials for the first electrode 101 and the second electrode 102, any of the following materials can be used in an appropriate combination as long as the above functions of the electrodes can be fulfilled. For example, a metal, an alloy, an electrically conductive compound, a mixture of these, and the like can be used as appropriate. Specifically, an In—Sn oxide (also referred to as ITO), an In—Si—Sn oxide (also referred to as ITSO), an In—Zn oxide, or an In—W—Zn oxide can be used. In addition, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use an element belonging to Group 1 or Group 2 of the periodic table that is not described above (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In the light-emitting device in FIG. 4D, when the first electrode 101 is the anode, a hole-injection layer 111a and a hole-transport layer 112a of the EL layer 103a are sequentially stacked over the first electrode 101 by a vacuum evaporation method. After the EL layer 103a and the charge-generation layer 106 are formed, a hole-injection layer 111b and a hole-transport layer 112b of the EL layer 103b are sequentially stacked over the charge-generation layer 106 in a similar manner.

### Hole-Injection Layer

The hole-injection layers (111, 111a, and 111b) inject holes from the first electrode 101 serving as the anode or the charge-generation layers (106, 106a, and 106b) to the EL layers (103, 103a, and 103b) and contain an organic acceptor material, a material having a high hole-injection property, and the like.

The organic acceptor material allows holes to be generated in another organic compound whose HOMO level is close to the LUMO level of the organic acceptor material when charge separation is caused between the organic acceptor material and the organic compound. Thus, as the organic acceptor material, a compound having an electron-withdrawing group (e.g., a halogen group or a cyano group), such as a quinodimethane derivative, a chloranil derivative, and a hexaazatriphenylene derivative, can be used. Examples of the organic acceptor material include 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F₆-TCNNQ), and 2-(7-dicyanomethylene-1,3,4,5,6,8,9,10-octafluoro-7H-pyren-2-ylidene)malononitrile. Note that among organic acceptor materials, a compound in which electron-withdrawing groups are bonded to fused aromatic rings each having a plurality of heteroatoms, such as HAT-CN, is particularly preferable because it has a high acceptor property and stable film quality against heat. Besides, a [3]radialene derivative having an electron-withdrawing group (particularly a cyano group or a halogen group such as a fluoro group), which has a very high electron-accepting property, is preferable; specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile].

As the material having a high hole-injection property, an oxide of a metal belonging to Group 4 to Group 8 of the periodic table (e.g., a transition metal oxide such as molybdenum oxide, vanadium oxide, ruthenium oxide, tungsten oxide, or manganese oxide) can be used. Specific examples include molybdenum oxide, vanadium oxide, niobium oxide, tantalum oxide, chromium oxide, tungsten oxide, manganese oxide, and rhenium oxide. Among these oxides, molybdenum oxide is preferable because it is stable in the air, has a low hygroscopic property, and is easily handled. Other examples include phthalocyanine (abbreviation: H₂Pc) and a phthalocyanine-based compound such as copper phthalocyanine (abbreviation: CuPc).

Other examples include aromatic amine compounds, which are low molecular compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), N,N′-bis[4-bis(3-methylphenyl)aminophenyl]-N,N-diphenyl-4,4′-diaminobiphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1).

Other examples include high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N-phenylamino}phenyl)methacrylamide] (abbreviation: PTPDMA), and poly[N,N-bis(4-butylphenyl)-N,N-bis(phenyl)benzidine] (abbreviation: Poly-TPD). Alternatively, it is possible to use a high-molecular compound to which acid is added, such as poly(3,4-ethylenedioxythiophene)/poly(styrenesulfonic acid) (abbreviation: PEDOT/PSS) or polyaniline/poly(styrenesulfonic acid) (abbreviation: PAni/PSS), for example.

As the material having a high hole-injection property, a mixed material containing a hole-transport material and the above-described organic acceptor material (electron-accepting material) can be used. In that case, the organic acceptor material extracts electrons from the hole-transport material, so that holes are generated in the hole-injection layer 111 and the holes are injected into the light-emitting layer 113 through the hole-transport layer 112. Note that the hole-injection layer 111 may be formed to have a single-layer structure using a mixed material containing a hole-transport material and an organic acceptor material (electron-accepting material), or a stacked-layer structure of a layer containing a hole-transport material and a layer containing an organic acceptor material (electron-accepting material).

The hole-transport material preferably has a hole mobility higher than or equal to 1×10⁻⁶ cm²/Vs in the case where the square root of the electric field strength [V/cm] is 600. Note that any other substance can also be used as long as the substance has a hole-transport property higher than an electron-transport property.

As the hole-transport material, materials having a high hole-transport property, such as a compound having a π-electron rich heteroaromatic ring (e.g., a carbazole derivative, a furan derivative, or a thiophene derivative) and an aromatic amine (an organic compound having an aromatic amine skeleton), are preferable.

Examples of the carbazole derivative (an organic compound having a carbazole ring) include a bicarbazole derivative (e.g., a 3,3′-bicarbazole derivative) and an aromatic amine having a carbazolyl group.

Specific examples of the bicarbazole derivative (e.g., a 3,3′-bicarbazole derivative) include 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(1,1′-biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), and 9-(2-naphthyl)-9′-phenyl-9H,9′H-3,3′-bicarbazole (abbreviation: βNCCP).

Specific examples of the aromatic amine having a carbazolyl group include 4-phenyl-4′-(9-phenyl-9H-carbazol-3-yl)triphenylamine (abbreviation: PCBA1BP), N-(4-biphenyl)-N-(9,9-dimethyl-9H-fluoren-2-yl)-9-phenyl-9H-carbazol-3-amine (abbreviation: PCBiF), N-(biphenyl-4-yl)-N-[4-(9-phenyl-9H-carbazol-3-yl)phenyl]-9,9-dimethyl-9H-fluoren-2-amine (abbreviation: PCBBiF), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N-bis(9-phenylcarbazol-3-yl)-N,N′-diphenylbenzene-1,3-diamine (abbreviation: PCA2B), N,N,N′-triphenyl-N,N′,N′-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]-9,9′-spirobi[9H-fluoren]-2-amine (abbreviation: PCBASF), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 2-[N-(9-phenylcarbazol-3-yl)-N-phenylamino]-9,9′-spirobi[9H-fluorene] (abbreviation: PCASF), N-[4-(9H-carbazol-9-yl)phenyl]-N-(4-phenyl)phenylaniline (abbreviation: YGA1BP), N,N-bis[4-(carbazol-9-yl)phenyl]-N,N-diphenyl-9,9-dimethylfluorene-2,7-diamine (abbreviation: YGA2F), and 4,4′,4″-tris(carbazol-9-yl)triphenylamine (abbreviation: TCTA).

Other examples of the carbazole derivative include 9-[4-(9-phenyl-9H-carbazole-3-yl)phenyl]phenanthrene (abbreviation: PCPPn), 3-[4-(1-naphthyl)phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA).

Specific examples of the furan derivative (an organic compound having a furan ring) include 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II).

Specific examples of the thiophene derivative (an organic compound having a thiophene ring) include organic compounds having a thiophene ring, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the aromatic amine include 4,4′-bis[N-(1-naphthyl)-N-phenylamino]biphenyl (abbreviation: NPB or α-NPD), N,N-diphenyl-N,N-bis(3-methylphenyl)-4,4′-diaminobiphenyl (abbreviation: TPD), N,N-bis(9,9′-spirobi[9H-fluoren]-2-yl)-N,N-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(4-biphenyl)-N-{4-[(9-phenyl)-9H-fluoren-9-yl]phenyl}-9,9-dimethyl-9H-fluoren-2-amine (abbreviation: FBiFLP), N,N,N,N-tetrakis(4-biphenyl)-1,1-biphenyl-4,4′-diamine (abbreviation: BBA2BP), N,N-bis(9,9-dimethyl-9H-fluoren-2-yl)-9,9′-spirobi[9H-fluoren]-4-amine (abbreviation: SF4FAF), N-(9,9-dimethyl-9H-fluoren-2-yl)-N-{9,9-dimethyl-2-[N¹-phenyl-N-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), 2-[N-(4-diphenylaminophenyl)-N-phenylamino]-9,9′-spirobi[9H-fluorene] (abbreviation: DPASF), 2,7-bis[N-(4-diphenylaminophenyl)-N-phenylamino]-9,9′-spirobi[9H-fluorene] (abbreviation: DPA2SF), 4,4′,4″-tris[N-(1-naphthyl)-N-phenylamino]triphenylamine (abbreviation: 1′-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), N,N-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), DNTPD, 1,3,5-tris[N-(4-diphenylaminophenyl)-N-phenylamino]benzene (abbreviation: DPA3B), N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6; 1′-binaphthyl-2-yl)triphenylamine (abbreviation: BBAαNβNB), 4,4′-diphenyl-4″-(7; 1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), bis-biphenyl-4′-(carbazol-9-yl)biphenylamine (abbreviation: YGBBi1BP), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Other examples of the hole-transport material include high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N-[4-(4-diphenylamino)phenyl]phenyl-N-phenylamino}phenyl)methacrylamide](abbreviation: PTPDMA), and poly[N,N-bis(4-butylphenyl)-N,N′-bis(phenyl)benzidine](abbreviation: Poly-TPD). Alternatively, it is possible to use a high-molecular compound to which acid is added, such as poly(3,4-ethylenedioxythiophene)/poly(styrenesulfonic acid) (abbreviation: PEDOT/PSS) or polyaniline/poly(styrenesulfonic acid) (abbreviation: PAni/PSS), for example.

Note that the hole-transport material is not limited to the above examples, and any of a variety of known materials may be used alone or in combination as the hole-transport material.

The hole-injection layers (111, 111a, and 111b) can be formed by any of known film formation methods such as a vacuum evaporation method.

### Hole-Transport Layer

The hole-transport layers (112, 112a, and 112b) transport the holes, which are injected from the first electrode 101 by the hole-injection layers (111, 111a, and 111b), to the light-emitting layers (113, 113a, and 113b). Note that the hole-transport layers (112, 112a, and 112b) contain a hole-transport material. Thus, the hole-transport layers (112, 112a, and 112b) can be formed using a hole-transport material that can be used for the hole-injection layers (111, 111a, and 111b).

Note that in the light-emitting device of one embodiment of the present invention, the organic compound used for the hole-transport layers (112, 112a, and 112b) can also be used for the light-emitting layers (113, 113a, and 113b). The use of the same organic compound for the hole-transport layers (112, 112a, and 112b) and the light-emitting layers (113, 113a, and 113b) is preferable, in which case holes can be efficiently transported from the hole-transport layers (112, 112a, and 112b) to the light-emitting layers (113, 113a, and 113b).

### Light-Emitting Layer

The light-emitting layers (113, 113a, and 113b) contain a light-emitting substance. Note that as a light-emitting substance that can be used in the light-emitting layers (113, 113a, and 113b), a substance whose emission color is blue, violet, bluish violet, green, yellowish green, yellow, orange, red, or the like can be used as appropriate. When a plurality of light-emitting layers are provided, the use of different light-emitting substances for the light-emitting layers enables a structure that exhibits different emission colors (e.g., white light emission obtained by a combination of complementary emission colors). Furthermore, one light-emitting layer may have a stacked-layer structure of layers containing different light-emitting substances.

The light-emitting layers (113, 113a, and 113b) may each contain one or more kinds of organic compounds (e.g., a host material) in addition to a light-emitting substance (guest material).

In the case where a plurality of host materials are used in the light-emitting layers (113, 113a, and 113b), a second host material that is additionally used is preferably a substance having a larger energy gap than a known guest material and a first host material. Preferably, the lowest singlet excitation energy level (S1 level) of the second host material is higher than that of the first host material, and the lowest triplet excitation energy level (T1 level) of the second host material is higher than that of the guest material. Preferably, the lowest triplet excitation energy level (T1 level) of the second host material is higher than that of the first host material. With such a structure, an exciplex can be formed by the two kinds of host materials. To form an exciplex efficiently, it is particularly preferable to combine a compound that easily accepts holes (hole-transport material) and a compound that easily accepts electrons (electron-transport material). With the above structure, high efficiency, a low voltage, and a long lifetime can be achieved at the same time.

As an organic compound used as the host material (including the first host material and the second host material), organic compounds such as the hole-transport materials usable in the hole-transport layers (112, 112a, and 112b) and electron-transport materials usable in electron-transport layers (114, 114a, and 114b) described later can be used as long as they satisfy requirements for the host material used in the light-emitting layer. Another example is an exciplex formed by two or more kinds of organic compounds (the first host material and the second host material). An exciplex whose excited state is formed by two or more kinds of organic compounds has an extremely small difference between the S1 level and the T1 level and functions as a TADF material capable of converting triplet excitation energy into singlet excitation energy. In an example of a preferable combination of two or more kinds of organic compounds forming an exciplex, one of the two or more kinds of organic compounds has a π-electron deficient heteroaromatic ring and the other has a π-electron rich heteroaromatic ring. A phosphorescent substance such as an iridium-, rhodium-, or platinum-based organometallic complex or a metal complex may be used as one component of the combination for forming an exciplex.

There is no particular limitation on the light-emitting substances that can be used for the light-emitting layers (113, 113a, and 113b), and a light-emitting substance that converts singlet excitation energy into light in the visible light range or a light-emitting substance that converts triplet excitation energy into light in the visible light range can be used.

### Light-Emitting Substance that Converts Singlet Excitation Energy into Light

The following substances that emit fluorescent light (fluorescent substances) can be given as examples of the light-emitting substance that converts singlet excitation energy into light and can be used in the light-emitting layers (113, 113a, and 113b): a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of pyrene derivatives include N,N-bis(3-methylphenyl)-N,N-bis[3-(9-phenyl-9H-fluoren-9-yl)phenyl]pyrene-1,6-diamine (abbreviation: 1,6mMemFLPAPrn), N,N-diphenyl-N,N′-bis[4-(9-phenyl-9H-fluoren-9-yl)phenyl]pyrene-1,6-diamine (abbreviation: 1,6FLPAPrn), N,N-bis(dibenzofuran-2-yl)-N,N-diphenylpyrene-1,6-diamine (abbreviation: 1,6FrAPrn), N,N-bis(dibenzothiophen-2-yl)-N,N-diphenylpyrene-1,6-diamine (abbreviation: 1,6ThAPrn), N,N-(pyrene-1,6-diyl)bis[(N-phenylbenzo[b]naphtho[1,2-d]furan)-6-amine] (abbreviation: 1,6BnfAPrn), N,N-(pyrene-1,6-diyl)bis[(N-phenylbenzo[b]naphtho[1,2-d]furan)-8-amine](abbreviation: 1,6BnfAPrn-02), and N,N-(pyrene-1,6-diyl)bis[(6,N-diphenylbenzo[b]naphtho[1,2-d]furan)-8-amine] (abbreviation: 1,6BnfAPrn-03).

In addition, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra(tert-butyl)perylene (abbreviation: TBP), N,N′-(2-tert-butylanthracene-9,10-diyldi-4,1-phenylene)bis(N,N,N-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), and N-[4-(9,10-diphenyl-2-anthryl)phenyl]-N,N′,N′-triphenyl-1,4-phenylenediamine (abbreviation: 2DPAPPA).

It is also possible to use, for example, N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N,N-triphenyl-1,4-phenylenediamine (abbreviation: 2DPAPA), N-[9,10-bis(biphenyl-2-yl)-2-anthryl]-N,N,N-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N,N-tetrakis(4-methylphenyl)tetracene-5,11-diamine (abbreviation: p-mPhTD), 7,14-diphenyl-N,N,N,N-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJ™), 1,6BnfAPrn-03, 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). In particular, pyrenediamine compounds such as 1,6FLPAPrn, 1,6mMemFLPAPrn, and 1,6BnfAPrn-03 can be used, for example.

### Light-Emitting Substance that Converts Triplet Excitation Energy into Light

Examples of the light-emitting substance that converts triplet excitation energy into light and can be used in the light-emitting layer 113 include substances that exhibit phosphorescent light (phosphorescent materials) and thermally activated delayed fluorescent (TADF) materials that exhibit thermally activated delayed fluorescence.

A phosphorescent substance is a compound that emits phosphorescent light but does not emit fluorescent light at a temperature higher than or equal to a low temperature (e.g., 77 K) and lower than or equal to room temperature (i.e., higher than or equal to 77 K and lower than or equal to 313 K). The phosphorescent substance preferably contains a metal element with large spin-orbit interaction, and can be an organometallic complex, a metal complex (platinum complex), or a rare earth metal complex, for example. Specifically, the phosphorescent substance preferably contains a transition metal element. It is particularly preferable that the phosphorescent substance contain a platinum group element (ruthenium (Ru), rhodium (Rh), palladium (Pd), osmium (Os), iridium (Ir), or platinum (Pt)), especially iridium, in which case the probability of direct transition between the singlet ground state and the triplet excited state can be increased.

### Phosphorescent Substance (from 450 nm to 570 nm, Blue or Green)

As examples of a phosphorescent substance which emits blue or green light and whose emission spectrum has a peak wavelength higher than or equal to 450 nm and lower than or equal to 570 nm, the following substances can be given.

Examples include organometallic complexes having a 4H-triazole ring, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole ring, such as fac-tris[l-(2,6-diisopropylphenyl)-2-phenyl-1H-imidazole]iridium(III) (abbreviation: [Ir(iPrpim)₃]) and tris[3-(2,6-dimethylphenyl)-7-methylimidazo[1,2-f]phenanthridinato]iridium(III) (abbreviation: [Ir(dmpimpt-Me)₃]); and organometallic complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)).

### Phosphorescent Substance (from 495 nm to 590 nm, Green or Yellow)

As examples of a phosphorescent substance which emits green or yellow light and whose emission spectrum has a peak wavelength higher than or equal to 495 nm and lower than or equal to 590 nm, the following substances can be given.

Examples include organometallic iridium complexes having a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN³]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine ring, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-κC][2-(4-methyl-5-phenyl-2-pyridinyl-κN)phenyl-κC], [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: [Ir(5mppy-d₃)₂(mbfpypy-d₃)]), [2-(methyl-d₃)-8-[4-(1-methylethyl-1-d)-2-pyridinyl-κN]benzofuro[2,3-b]pyridin-7-yl-κC]bis[5-(methyl-d₃)-2-[5-(methyl-d₃)-2-pyridinyl-N]phenyl-κC]iridium(III) (abbreviation: Ir(5mtpy-d₆)₂(mbfpypy-iPr-d₄)), [2-d₃-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d₃)), and [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)); organometallic complexes such as bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C²}iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); and a rare earth metal complex such as tris(acetylacetonato) (monophenanthroline)terbium(III) (abbreviation: [Tb(acac)₃(Phen)]).

### Phosphorescent Substance (from 570 nm to 750 nm, Yellow or Red)

As examples of a phosphorescent substance which emits yellow or red light and whose emission spectrum has a peak wavelength higher than or equal to 570 nm and lower than or equal to 750 nm, the following substances can be given.

Examples include organometallic complexes having a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-dmCP)₂(dpm)]), bis[2-(5-(2,6-dimethylphenyl)-3-(3,5-dimethylphenyl)-2-pyrazinyl-κN)-4,6-dimethylphenyl-κC](2,2′, 6,6′-tetramethyl-3,5-heptanedionato-κ²O,O′)iridium(III) (abbreviation: [Ir(dmdppr-dmp)₂(dpm)]), (acetylacetonato)bis[2-methyl-3-phenylquinoxalinato-N,C^{2′}]iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine ring, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), and bis[4,6-dimethyl-2-(2-quinolinyl-κN)phenyl-κC](2,4-pentanedionato-κ²O,O′)iridium(III) (abbreviation: [Ir(dmpqn)₂(acac)]); a platinum complex such as 2,3,7,8,12,13,17,18-octaethyl-21H,23H-porphyrin platinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]).

### TADF Material

Any of materials described below can be used as the TADF material. The TADF material is a material that has a small difference between its S1 and T1 levels (preferably less than or equal to 0.2 eV), enables up-conversion of a triplet excited state into a singlet excited state (i.e., reverse intersystem crossing) using a little thermal energy, and efficiently emits light (fluorescent light) from the singlet excited state. The thermally activated delayed fluorescence is efficiently obtained under the condition where the difference in energy between the triplet excited energy level and the singlet excited energy level is greater than or equal to 0 eV and less than or equal to 0.2 eV, preferably greater than or equal to 0 eV and less than or equal to 0.1 eV. Note that delayed fluorescence by the TADF material refers to light emission having a spectrum similar to that of normal fluorescent light and an extremely long lifetime. The lifetime is longer than or equal to 1×10⁻⁶ seconds, preferably longer than or equal to 1×10⁻³ seconds.

Examples of the TADF material include fullerene, a derivative thereof, an acridine derivative such as proflavine, and eosin. Other examples include a metal-containing porphyrin such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

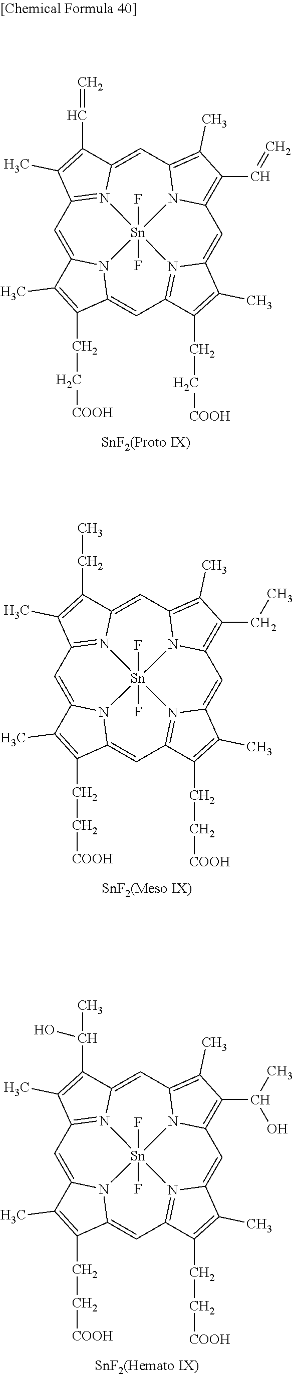

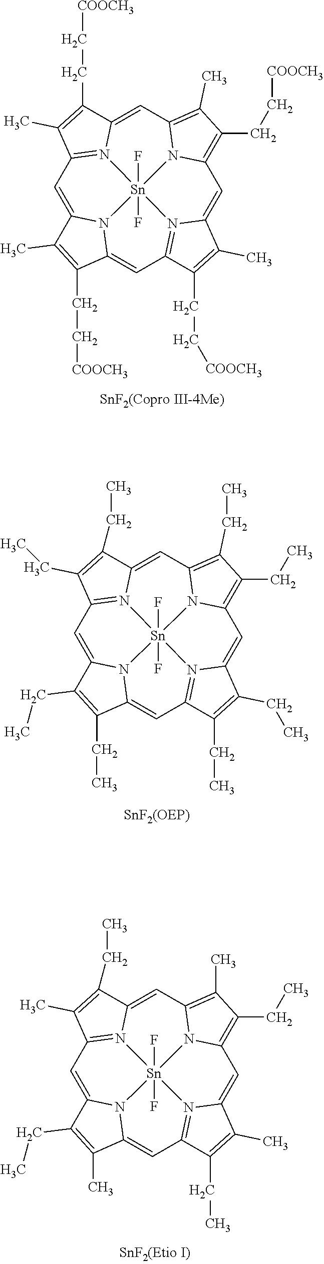

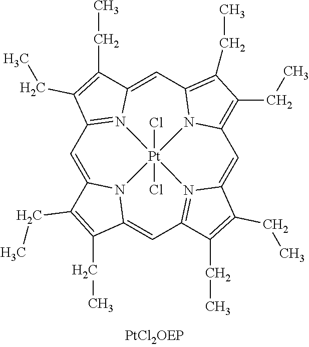

Alternatively, a heteroaromatic compound including a π-electron rich heteroaromatic compound and a π-electron deficient heteroaromatic compound, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-1OH,10′H-spiro[acridin-9,9′-anthracen]-10′-one (abbreviation: ACRSA), 4-(9′-phenyl-3,3′-bi-9H-carbazol-9-yl)benzofuro[3,2-d]pyrimidine (abbreviation: 4PCCzBfpm), 4-[4-(9′-phenyl-3,3′-bi-9H-carbazol-9-yl)phenyl]benzofuro[3,2-d]pyrimidine (abbreviation: 4PCCzPBfpm), or 9-[3-(4,6-diphenyl-1,3,5-triazin-2-yl)phenyl]-9′-phenyl-2,3′-bi-9H-carbazole (abbreviation: mPCCzPTzn-02) may be used.

Note that a substance in which a π-electron rich heteroaromatic compound is directly bonded to a π-electron deficient heteroaromatic compound is particularly preferable because both the donor property of the π-electron rich heteroaromatic compound and the acceptor property of the π-electron deficient heteroaromatic compound are improved and the energy difference between the singlet excited state and the triplet excited state becomes small. As the TADF material, a TADF material in which the singlet and triplet excited states are in thermal equilibrium (TADF100) may be used. Since such a TADF material enables a short emission lifetime (excitation lifetime), an efficiency decrease of a light-emitting device in a high-luminance region can be inhibited.

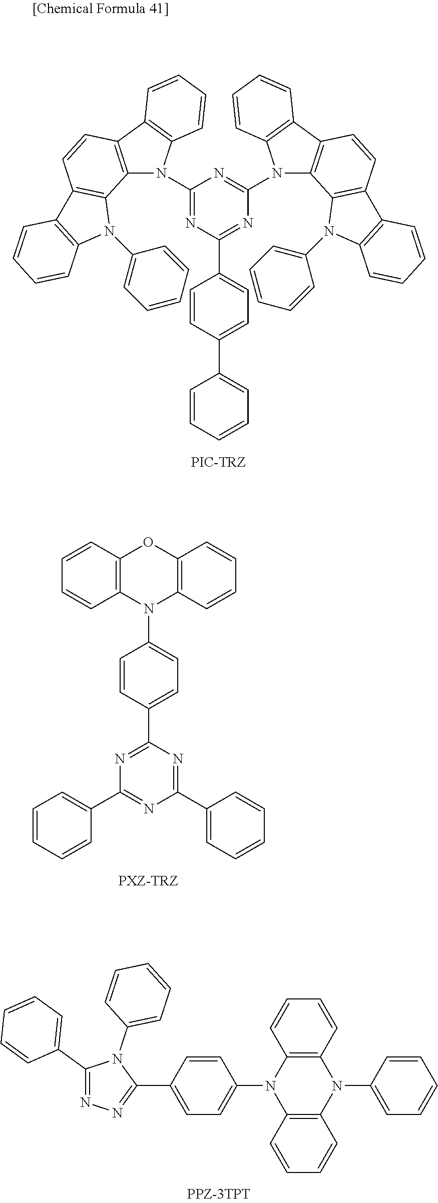 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1]

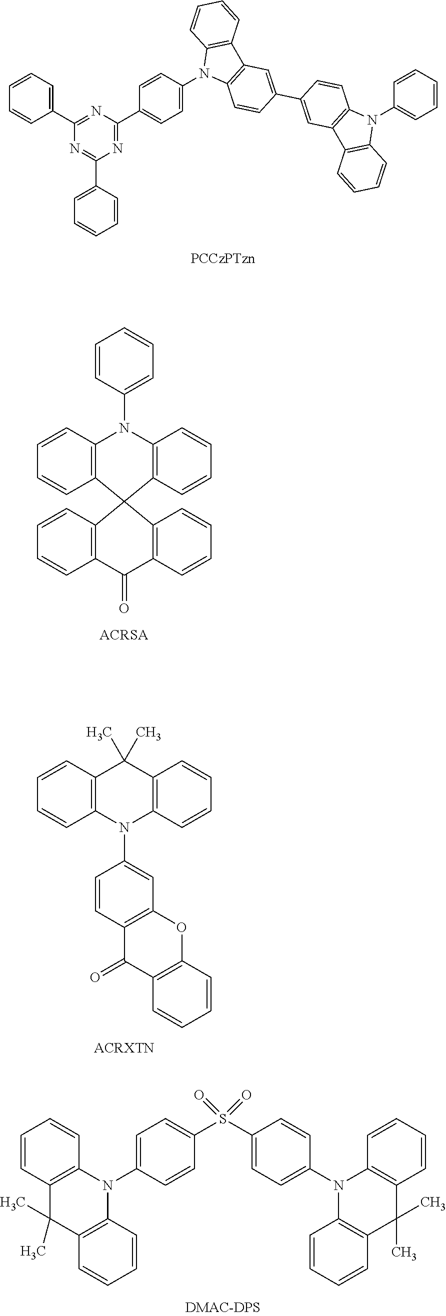 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; O=C1c2ccccc2C2(c3ccccc31)c1ccccc1N(c1ccccc1)c1ccccc12 ; CC1(C)c2ccccc2N(c2ccc3c(=O)c4ccccc4oc3c2)c2ccccc21 ; CC1(C)c2ccccc2N(c2ccc(S(=O)(=O)c3ccc(N4c5ccccc5C(C)(C)c5ccccc54)cc3)cc2)c2ccccc21]

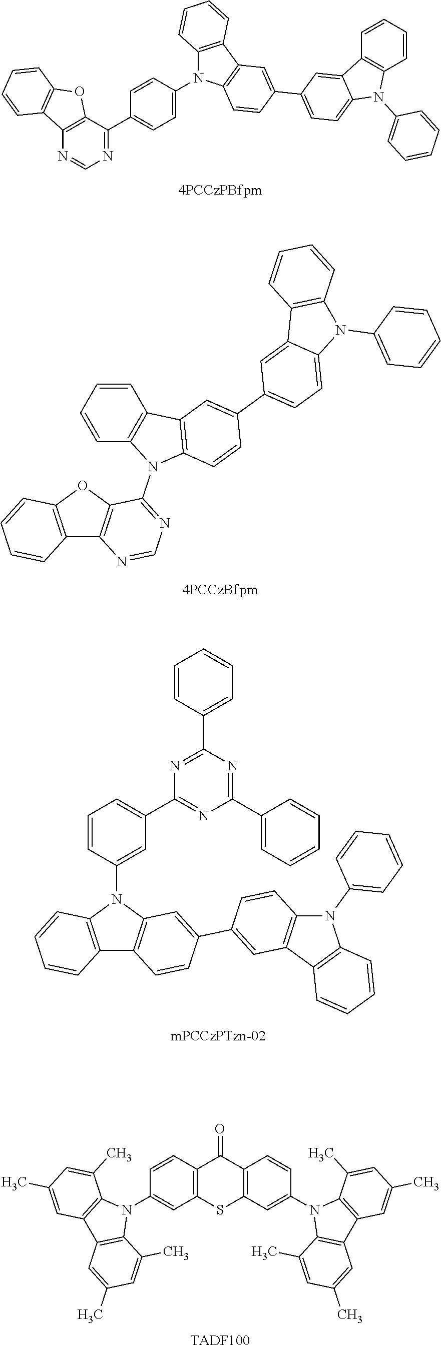

In addition to the above, another example of a material having a function of converting triplet excitation energy into light is a nano-structure of a transition metal compound having a perovskite structure. In particular, a nano-structure of a metal halide perovskite material is preferable. The nano-structure is preferably a nanoparticle or a nanorod.

As the organic compound (e.g., the host material) used in combination with the above-described light-emitting substance (guest material) in the light-emitting layers (113, 113a, 113b, and 113c), one or more kinds selected from substances having a larger energy gap than the light-emitting substance (guest material) are used.

### Host Material for Fluorescence

In the case where the light-emitting substance used in the light-emitting layers (113, 113a, 113b, and 113c) is a fluorescent substance, an organic compound (a host material) used in combination with the fluorescent substance is preferably an organic compound that has a high energy level in a singlet excited state and has a low energy level in a triplet excited state, or an organic compound having a high fluorescence quantum yield. Therefore, the hole-transport material (described above) or the electron-transport material (described below) described in this embodiment, for example, can be used as long as it is an organic compound that satisfies such a condition.

In terms of a preferable combination with the light-emitting substance (fluorescent substance), examples of the organic compound (host material), some of which overlap the above specific examples, include fused polycyclic aromatic compounds such as an anthracene derivative, a tetracene derivative, a phenanthrene derivative, a pyrene derivative, a chrysene derivative, and a dibenzo[g,p]chrysene derivative.

Specific examples of the organic compound (host material) that is preferably used in combination with the fluorescent substance include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), 3-[4-(1-naphthyl)phenyl]-9-phenyl-9H-carbazole (abbreviation: PCPN), 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N,N,N′,N′,N″,N″-octaphenyldibenzo[g,p]chrysene-2,7,10,15-tetraamine (abbreviation: DBC1), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-{4-(9-phenyl-9H-fluoren-9-yl)biphenyl-4′-yl}anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9-(1-naphthyl)-10-(2-naphthyl)anthracene (abbreviation: a,P-ADN), 2-(10-phenylanthracen-9-yl)dibenzofuran, 2-(10-phenyl-9-anthracenyl)benzo[b]naphtho[2,3-d]furan (abbreviation: Bnf(II)PhA), 9-(1-naphthyl)-10-[4-(2-naphthyl)phenyl]anthracene (abbreviation: αN-PNPAnth), 9-(2-naphthyl)-10-[3-(2-naphthyl)phenyl]anthracene (abbreviation: βN-mPNPAnth), 1-[4-(10-biphenyl-4-yl-9-anthracenyl)phenyl]-2-ethyl-1H-benzimidazole (abbreviation: EtBImPBPhA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, and 5,12-bis(biphenyl-2-yl)tetracene.

### Host Material for Phosphorescence

In the case where the light-emitting substance used in the light-emitting layers (113, 113a, 113b, and 113c) is a phosphorescent substance, an organic compound having triplet excitation energy (an energy difference between a ground state and a triplet excited state) which is higher than that of the light-emitting substance is preferably selected as the organic compound (host material) used in combination with the phosphorescent substance. Note that when a plurality of organic compounds (e.g., a first host material and a second host material (or an assist material)) are used in combination with a light-emitting substance so that an exciplex is formed, the plurality of organic compounds are preferably mixed with the phosphorescent substance.

With such a structure, light emission can be efficiently obtained by exciplex-triplet energy transfer (ExTET), which is energy transfer from an exciplex to a light-emitting substance. Note that a combination of the plurality of organic compounds that easily forms an exciplex is preferably employed, and it is particularly preferable to combine a compound that easily accepts holes (hole-transport material) and a compound that easily accepts electrons (electron-transport material).

In terms of a preferable combination with the light-emitting substance (phosphorescent substance), examples of the organic compounds (the host material and the assist material), some of which overlap the above specific examples, include an aromatic amine (an organic compound having an aromatic amine skeleton), a carbazole derivative (an organic compound having a carbazole ring), a dibenzothiophene derivative (an organic compound having a dibenzothiophene ring), a dibenzofuran derivative (an organic compound having a dibenzofuran ring), an oxadiazole derivative (an organic compound having an oxadiazole ring), a triazole derivative (an organic compound having a triazole ring), a benzimidazole derivative (an organic compound having a benzimidazole ring), a quinoxaline derivative (an organic compound having a quinoxaline ring), a dibenzoquinoxaline derivative (an organic compound having a dibenzoquinoxaline ring), a pyrimidine derivative (an organic compound having a pyrimidine ring), a triazine derivative (an organic compound having a triazine ring), a pyridine derivative (an organic compound having a pyridine ring), a bipyridine derivative (an organic compound having a bipyridine ring), a phenanthroline derivative (an organic compound having a phenanthroline ring), a furodiazine derivative (an organic compound having a furodiazine ring), and zinc- and aluminum-based metal complexes.

Among the above organic compounds, specific examples of the aromatic amine and the carbazole derivative, which are organic compounds having a high hole-transport property, are the same as the specific examples of the hole-transport materials described above, and those materials are preferable as the host material.

Among the above organic compounds, specific examples of the dibenzothiophene derivative and the dibenzofuran derivative, which are organic compounds having a high hole-transport property, include 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), DBT3P-II, 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II). Such derivatives are preferable as the host material.

Other examples of preferable host materials include metal complexes having an oxazole-based or thiazole-based ligand, such as bis[2-(2-benzoxazolyl)phenolato]zinc(II) (abbreviation: ZnPBO) and bis[2-(2-benzothiazolyl)phenolato]zinc(II) (abbreviation: ZnBTZ).

Among the above organic compounds, specific examples of the oxadiazole derivative, the triazole derivative, the benzimidazole derivative, the quinoxaline derivative, the dibenzoquinoxaline derivative, the quinazoline derivative, and the phenanthroline derivative, which are organic compounds having a high electron-transport property, include an organic compound including a heteroaromatic ring having a polyazole ring, such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or 4,4′-bis(5-methylbenzoxazol-2-yl)stilbene (abbreviation: BzOs), an organic compound including a heteroaromatic ring having a pyridine ring, such as bathophenanthroline (abbreviation: BPhen), bathocuproine (abbreviation: BCP), 2,9-di(naphthalen-2-yl)-4,7-diphenyl-1,10-phenanthroline (abbreviation: NBPhen), 2,2′-(1,3-phenylene)bis(9-phenyl-1,10-phenanthroline) (abbreviation: mPPhen2P), or 2-phenyl-9-{4-[4-(9-phenyl-1,10-phenanthrolin-2-yl)phenyl]phenyl}-1,10-phenanthroline (abbreviation: PPhen2BP), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 2-{4-[9,10-di(2-naphthyl)-2-anthryl]phenyl}-1-phenyl-1H-benzimidazole (abbreviation: ZADN), and 2-[4′-(9-phenyl-9H-carbazol-3-yl)-3,1′-biphenyl-1-yl]dibenzo[f,h]quinoxaline (abbreviation: 2mpPCBPDBq). Such organic compounds are preferable as the host material.

Among the above organic compounds, specific examples of the pyridine derivative, the diazine derivative (including the pyrimidine derivative, the pyrazine derivative, and the pyridazine derivative), the triazine derivative, and the furodiazine derivative, which are organic compounds having a high electron-transport property, include organic compounds including a heteroaromatic ring having a diazine ring, such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), 9,9′-[pyrimidine-4,6-diylbis(biphenyl-3,3′-diyl)]bis(9H-carbazole) (abbreviation: 4,6mCzBP2Pm), 2-[3′-(9,9-dimethyl-9H-fluoren-2-yl)biphenyl-3-yl]-4,6-diphenyl-1,3,5-triazine (abbreviation: mFBPTzn), 8-(biphenyl-4-yl)-4-[3-(dibenzothiophen-4-yl)phenyl]-[1]benzofuro[3,2-d]pyrimidine (abbreviation: 8BP-4mDBtPBfpm), 9-[3′-(dibenzothiophen-4-yl)biphenyl-3-yl]naphtho[1′,2′:4,5]furo[2,3-b]pyrazine (abbreviation: 9mDBtBPNfpr), 9-[(3′-dibenzothiophen-4-yl)biphenyl-4-yl]naphtho[1′,2′:4,5]furo[2,3-b]pyrazine (abbreviation: 9pmDBtBPNfpr), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-{8-[(1,1′:4′,1″-terphenyl)-4-yl]-1-dibenzofuranyl}-1,3,5-triazine (abbreviation: mBP-TPDBfTzn), 6-(biphenyl-3-yl)-4-[3,5-bis(9H-carbazol-9-yl)phenyl]-2-phenylpyrimidine (abbreviation: 6mBP-4Cz2PPm), and 4-[3,5-bis(9H-carbazol-9-yl)phenyl]-2-phenyl-6-(biphenyl-4-yl)pyrimidine (abbreviation: 6BP-4Cz2PPm). Such organic compounds are preferable as the host material.

Among the above organic compounds, specific examples of metal complexes that are organic compounds having a high electron-transport property include zinc- and aluminum-based metal complexes, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), and bis(8-quinolinolato)zinc(II) (abbreviation: Znq), and metal complexes having a quinoline ring or a benzoquinoline ring. Such metal complexes are preferable as the host material.

Moreover, high molecular compounds such as poly(2,5-pyridinediyl) (abbreviation: PPy), poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)] (abbreviation: PF-BPy) are preferable as the host material.

Examples of organic compounds having bipolar properties, a high hole-transport property and a high electron-transport property, which can be used as the host material, include organic compounds having a diazine ring, such as 9-phenyl-9′-(4-phenyl-2-quinazolinyl)-3,3′-bi-9H-carbazole (abbreviation: PCCzQz), 2-[4′-(9-phenyl-9H-carbazol-3-yl)-3,1′-biphenyl-1-yl]dibenzo[f,h]quinoxaline (abbreviation: 2mpPCBPDBq), 5-[3-(4,6-diphenyl-1,3,5-triazin-2-yl)phenyl]-7,7-dimethyl-5H,7H-indeno[2,1-b]carbazole (abbreviation: mINc(II)PTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), and 7-[4-(9-phenyl-9H-carbazol-2-yl)quinazolin-2-yl]-7H-dibenzo[c,g]carbazole (abbreviation: PC-cgDBCzQz).

### Electron-Transport Layer

The electron-transport layers (114, 114a, and 114b) transport the electrons, which are injected from the second electrode 102 or the charge-generation layers (106, 106a, and 106b) by electron-injection layers (115, 115a, and 115b) described later, to the light-emitting layers (113, 113a, and 113b). It is preferable that the electron-transport material used in the electron-transport layers (114, 114a, and 114b) be a substance having an electron mobility higher than or equal to 1×10⁻⁶ cm²/Vs in the case where the square root of the electric field strength [V/cm] is 600. Note that any other substance can also be used as long as the substance has an electron-transport property higher than a hole-transport property. The electron-transport layers (114, 114a, and 114b) can function even with a single-layer structure and may have a stacked-layer structure including two or more layers. A photolithography process performed over the electron-transport layer including the above-described mixed material, which has heat resistance, can inhibit an adverse effect of the thermal process on the device characteristics.

### Electron-Transport Material

As the electron-transport material that can be used for the electron-transport layers (114, 114a, and 114b), an organic compound having a high electron-transport property can be used, and for example, a heteroaromatic compound can be used. The heteroaromatic compound refers to a cyclic compound containing at least two different kinds of elements in a ring. Examples of cyclic structures include a three-membered ring, a four-membered ring, a five-membered ring, and a six-membered ring, among which a five-membered ring and a six-membered ring are particularly preferable. The elements contained in the heteroaromatic compound are preferably one or more of nitrogen, oxygen, and sulfur, in addition to carbon. In particular, a heteroaromatic compound containing nitrogen (a nitrogen-containing heteroaromatic compound) is preferable, and any of materials having a high electron-transport property (electron-transport materials), such as a nitrogen-containing heteroaromatic compound and a π-electron deficient heteroaromatic compound including the nitrogen-containing heteroaromatic compound, is preferably used.

The heteroaromatic compound is an organic compound having at least one heteroaromatic ring.

The heteroaromatic ring has any one of a pyridine ring, a diazine ring, a triazine ring, a polyazole ring, an oxazole ring, a thiazole ring, and the like. A heteroaromatic ring having a diazine ring includes a heteroaromatic ring having a pyrimidine ring, a pyrazine ring, a pyridazine ring, or the like. A heteroaromatic ring having a polyazole ring includes a heteroaromatic ring having an imidazole ring, a triazole ring, or an oxadiazole ring.

The heteroaromatic ring includes a fused heteroaromatic ring having a fused ring structure. Examples of the fused heteroaromatic ring include a quinoline ring, a benzoquinoline ring, a quinoxaline ring, a dibenzoquinoxaline ring, a quinazoline ring, a benzoquinazoline ring, a dibenzoquinazoline ring, a phenanthroline ring, a furodiazine ring, and a benzimidazole ring.

Examples of the heteroaromatic compound having a five-membered ring structure, which is a heteroaromatic compound containing carbon and one or more of nitrogen, oxygen, sulfur, and the like, include a heteroaromatic compound having an imidazole ring, a heteroaromatic compound having a triazole ring, a heteroaromatic compound having an oxazole ring, a heteroaromatic compound having an oxadiazole ring, a heteroaromatic compound having a thiazole ring, and a heteroaromatic compound having a benzimidazole ring.

Examples of the heteroaromatic compound having a six-membered ring structure, which is a heteroaromatic compound containing carbon and one or more of nitrogen, oxygen, sulfur, and the like, include a heteroaromatic compound having a heteroaromatic ring, such as a pyridine ring, a diazine ring (including a pyrimidine ring, a pyrazine ring, a pyridazine ring, or the like), a triazine ring, or a polyazole ring. Other examples include a heteroaromatic compound having a bipyridine structure and a heteroaromatic compound having a terpyridine structure, although they are included in examples of a heteroaromatic compound in which pyridine rings are connected.

Examples of the heteroaromatic compound having a fused ring structure including the above six-membered ring structure as a part include a heteroaromatic compound having a fused heteroaromatic ring such as a quinoline ring, a benzoquinoline ring, a quinoxaline ring, a dibenzoquinoxaline ring, a phenanthroline ring, a furodiazine ring (including a structure in which an aromatic ring is fused to a furan ring of a furodiazine ring), or a benzimidazole ring.

Specific examples of the above-described heteroaromatic compound having a five-membered ring structure (a polyazole ring (including an imidazole ring, a triazole ring, or an oxadiazole ring), an oxazole ring, a thiazole ring, or a benzimidazole ring) include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs).

Specific examples of the above-described heteroaromatic compound having a six-membered ring structure (including a heteroaromatic ring having a pyridine ring, a diazine ring, a triazine ring, or the like) include a heteroaromatic compound including a heteroaromatic ring having a pyridine ring, such as 3,5-bis[3-(9H-carbazol-9-yl)phenyl]pyridine (abbreviation: 35DCzPPy) or 1,3,5-tri[3-(3-pyridyl)phenyl]benzene (abbreviation: TmPyPB), a heteroaromatic compound including a heteroaromatic ring having a triazine ring, such as 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-{8-[(1,1′:4′,1″-terphenyl)-4-yl]-1-dibenzofuranyl}-1,3,5-triazine (abbreviation: mBP-TPDBfTzn), 2-{3-[3-(dibenzothiophen-4-yl)phenyl]phenyl}-4,6-diphenyl-1,3,5-triazine (abbreviation: mDBtBPTzn), or mFBPTzn, and a heteroaromatic compound including a heteroaromatic ring having a diazine (pyrimidine) ring, such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 4,6mCzBP2Pm, 6-(biphenyl-3-yl)-4-[3,5-bis(9H-carbazol-9-yl)phenyl)-2-phenylpyrimidine (abbreviation: 6mBP-4Cz2PPm), 4-[3,5-bis(9H-carbazol-9-yl)phenyl]-2-phenyl-6-(biphenyl-4-yl)pyrimidine (abbreviation: 6BP-4Cz2PPm), 4-[3-(dibenzothiophen-4-yl)phenyl]-8-(naphthalen-2-yl)-[1]benzofuro[3,2-d]pyrimidine (abbreviation: 8βN-4mDBtPBfpm), 8BP-4mDBtPBfpm, 9mDBtBPNfpr, 9pmDBtBPNfpr, 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)(biphenyl-3-yl)]naphtho[1′, 2′: 4,5]furo[3,2-d]pyrimidine (abbreviation: 8mDBtBPNfpm), or 8-[(2,2′-binaphthalen)-6-yl]-4-[3-(dibenzothiophen-4-yl)phenyl]-[1]benzofuro[3,2-d]pyrimidine (abbreviation: 8(PN2)-4mDBtPBfpm). Note that the above aromatic compounds including a heteroaromatic ring include a heteroaromatic compound having a fused heteroaromatic ring.

Other examples include a heteroaromatic compound including a heteroaromatic ring having a diazine (pyrimidine) ring, such as 2,2′-(pyridine-2,6-diyl)bis(4-phenylbenzo[h]quinazoline) (abbreviation: 2,6(P-Bqn)2Py), 2,2′-(2,2′-bipyridine-6,6′-diyl)bis(4-phenylbenzo[h]quinazoline) (abbreviation: 6,6′(P-Bqn)2BPy), 2,2′-(pyridine-2,6-diyl)bis{4-[4-(2-naphthyl)phenyl]-6-phenylpyrimidine} (abbreviation: 2,6(NP-PPm)2Py), or 6-(biphenyl-3-yl)-4-[3,5-bis(9H-carbazol-9-yl)phenyl]-2-phenylpyrimidine (abbreviation: 6mBP-4Cz2PPm), and a heteroaromatic compound including a heteroaromatic ring having a triazine ring, such as 2,4,6-tris[3′-(pyridin-3-yl)biphenyl-3-yl]-1,3,5-triazine (abbreviation: TmPPPyTz), 2,4,6-tris(2-pyridyl)-1,3,5-triazine (abbreviation: 2Py3Tz), or 2-[3-(2,6-dimethyl-3-pyridyl)-5-(9-phenanthryl)phenyl]-4,6-diphenyl-1,3,5-triazine (abbreviation: mPn-mDMePyPTzn).

Specific examples of the above-described heteroaromatic compound having a fused ring structure including the above six-membered ring structure as a part (a heteroaromatic compound having a fused ring structure) include a heteroaromatic compound having a quinoxaline ring, such as bathophenanthroline (abbreviation: BPhen), bathocuproine (abbreviation: BCP), 2,9-di(naphthalen-2-yl)-4,7-diphenyl-1,10-phenanthroline (abbreviation: NBPhen), 2,2′-(1,3-phenylene)bis(9-phenyl-1,10-phenanthroline) (abbreviation: mPPhen2P), 2-phenyl-9-{4-[4-(9-phenyl-1,10-phenanthrolin-2-yl)phenyl]phenyl}-1,10-phenanthroline (abbreviation: PPhen2BP), 2,2′-(pyridine-2,6-diyl)bis(4-phenylbenzo[h]quinazoline) (abbreviation: 2,6(P-Bqn)2Py), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), or 2mpPCBPDBq.

For the electron-transport layers (114, 114a, and 114b), any of the metal complexes given below as well as the heteroaromatic compounds described above can be used. Examples of the metal complexes include a metal complex having a quinoline ring or a benzoquinoline ring, such as tris(8-quinolinolato)aluminum(III) (abbreviation: Alq₃), Almq₃, 8-(quinolinolato)lithium (abbreviation: Liq), BeBq₂, bis(2-methyl-8-quinolinolato) (4-phenylphenolato)aluminum(III) (abbreviation: BAlq), or bis(8-quinolinolato)zinc(II) (abbreviation: Znq), and a metal complex having an oxazole ring or a thiazole ring, such as bis[2-(2-benzoxazolyl)phenolato]zinc(II) (abbreviation: ZnPBO) or bis[2-(2-benzothiazolyl)phenolato]zinc(II) (abbreviation: ZnBTZ).

High-molecular compounds such as poly(2,5-pyridinediyl) (abbreviation: PPy), poly[(9,9-dihexylfluorene-2,7-diyl)-co-(pyridine-3,5-diyl)] (abbreviation: PF-Py), and poly[(9,9-dioctylfluorene-2,7-diyl)-co-(2,2′-bipyridine-6,6′-diyl)] (abbreviation: PF-BPy) can be used as the electron-transport material.

Each of the electron-transport layers (114, 114a, and 114b) is not limited to a single layer and may be a stack of two or more layers each containing any of the above substances.

### Electron-Injection Layer

The electron-injection layers (115, 115a, and 115b) contain a substance having a high electron-injection property. The electron-injection layers (115, 115a, and 115b) are layers for increasing the efficiency of electron injection from the second electrode 102 and are preferably formed using a material whose value of the LUMO level has a small difference (0.5 eV or less) from the work function of a material used for the second electrode 102. Thus, the electron-injection layer 115 can be formed using an alkali metal, an alkaline earth metal, or a compound thereof, such as lithium, cesium, lithium fluoride (LiF), cesium fluoride (CsF), calcium fluoride (CaF₂), 8-quinolinolato-lithium (abbreviation: Liq), 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), an oxide of lithium (LiOₓ), or cesium carbonate. A rare earth metal and a compound thereof such as erbium fluoride (ErF₃) and ytterbium (Yb) can also be used. To form the electron-injection layers (115, 115a, and 115b), a plurality of kinds of materials given above may be mixed or stacked. Electride may also be used for the electron-injection layers (115, 115a, and 115b). Examples of the electride include a substance in which electrons are added at high concentration to calcium oxide-aluminum oxide. Any of the substances used for the electron-transport layers (114, 114a, and 114b), which are given above, can also be used.

A mixed material in which an organic compound and an electron donor (donor) are mixed may also be used for the electron-injection layers (115, 115a, and 115b). Such a mixed material is excellent in an electron-injection property and an electron-transport property because electrons are generated in the organic compound by the electron donor. The organic compound here is preferably a material excellent in transporting the generated electrons; specifically, for example, the above-described electron-transport materials used for the electron-transport layers (114, 114a, and 114b), such as a metal complex and a heteroaromatic compound, can be used. As the electron donor, a substance showing an electron-donating property with respect to an organic compound is used. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable; for example, lithium oxide, calcium oxide, barium oxide, and the like are given. Alternatively, a Lewis base such as magnesium oxide can be used. Further alternatively, an organic compound such as tetrathiafulvalene (abbreviation: TTF) can be used. Alternatively, a stack of two or more of these materials may be used.

A mixed material in which an organic compound and a metal are mixed may also be used for the electron-injection layers (115, 115a, and 115b). The organic compound used here preferably has a lowest unoccupied molecular orbital (LUMO) level higher than or equal to −3.6 eV and lower than or equal to −2.3 eV. Moreover, a material having an unshared electron pair is preferable.

Thus, as the organic compound used in the above mixed material, a mixed material obtained by mixing a metal and the heteroaromatic compound given above as the material that can be used for the electron-transport layer may be used. Preferable examples of the heteroaromatic compound include materials having an unshared electron pair, such as a heteroaromatic compound having a five-membered ring structure (e.g., an imidazole ring, a triazole ring, an oxazole ring, an oxadiazole ring, a thiazole ring, or a benzimidazole ring), a heteroaromatic compound having a six-membered ring structure (e.g., a pyridine ring, a diazine ring (including a pyrimidine ring, a pyrazine ring, a pyridazine ring, or the like), a triazine ring, a bipyridine ring, or a terpyridine ring), and a heteroaromatic compound having a fused ring structure including a six-membered ring structure as a part (e.g., a quinoline ring, a benzoquinoline ring, a quinoxaline ring, a dibenzoquinoxaline ring, or a phenanthroline ring). Since the materials are specifically described above, description thereof is omitted here.

As a metal used for the above mixed material, a transition metal belonging to Group 5, Group 7, Group 9, or Group 11 or a material belonging to Group 13 of the periodic table is preferably used, and examples include Ag, Cu, Al, and In. Here, the organic compound forms a singly occupied molecular orbital (SOMO) with the transition metal.

To amplify light obtained from the light-emitting layer 113b, for example, the optical path length between the second electrode 102 and the light-emitting layer 113b is preferably less than one fourth of the wavelength k of light emitted from the light-emitting layer 113b. In that case, the optical path length can be adjusted by changing the thickness of the electron-transport layer 114b or the electron-injection layer 115b.

When the charge-generation layer 106 is provided between the two EL layers (103a and 103b) as in the light-emitting device in FIG. 4D, a structure in which a plurality of EL layers are stacked between the pair of electrodes (the structure is also referred to as a tandem structure) can be obtained.

### Charge-Generation Layer

The charge-generation layer 106 has a function of injecting electrons into the EL layer 103a and injecting holes into the EL layer 103b when voltage is applied between the first electrode (anode) 101 and the second electrode (cathode) 102. The charge-generation layer 106 may have either a structure in which an electron acceptor (acceptor) is added to a hole-transport material or a structure in which an electron donor (donor) is added to an electron-transport material. Alternatively, both of these layers may be stacked. Note that forming the charge-generation layer 106 with the use of any of the above materials can inhibit an increase in driving voltage caused by the stack of the EL layers.

In the case where the charge-generation layer 106 has a structure in which an electron acceptor is added to a hole-transport material, which is an organic compound, any of the materials described in this embodiment can be used as the hole-transport material. Examples of the electron acceptor include 7,7,8,8-tetracyano-2,3,5,6-tetrafluoroquinodimethane (abbreviation: F₄-TCNQ) and chloranil. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide.

In the case where the charge-generation layer 106 has a structure in which an electron donor is added to an electron-transport material, any of the materials described in this embodiment can be used as the electron-transport material. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide, cesium carbonate, or the like is preferably used. An organic compound such as tetrathianaphthacene may be used as the electron donor.

Although FIG. 4D illustrates the structure in which two EL layers 103 are stacked, three or more EL layers may be stacked with charge-generation layers each provided between two adjacent EL layers.

### Substrate

The light-emitting device described in this embodiment can be formed over a variety of substrates. Note that the type of substrate is not limited to a certain type. Examples of the substrate include semiconductor substrates (e.g., a single crystal substrate and a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, paper including a fibrous material, and a base material film.

Examples of the glass substrate include a barium borosilicate glass substrate, an aluminoborosilicate glass substrate, and a soda lime glass substrate. Examples of the flexible substrate, the attachment film, and the base material film include plastics typified by polyethylene terephthalate (PET), polyethylene naphthalate (PEN), and polyether sulfone (PES), a synthetic resin such as acrylic resin, polypropylene, polyester, polyvinyl fluoride, polyvinyl chloride, polyamide, polyimide, aramid, an epoxy resin, an inorganic vapor deposition film, and paper.

For fabrication of the light-emitting device in this embodiment, a gas phase method such as an evaporation method or a liquid phase method such as a spin coating method or an ink-jet method can be used. When an evaporation method is used, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, the layers having various functions (the hole-injection layer 111, the hole-transport layer 112, the light-emitting layer 113, the electron-transport layer 114, and the electron-injection layer 115) included in the EL layers of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, screen printing (stencil), offset printing (planography), flexography (relief printing), gravure printing, or micro-contact printing), or the like.

In the case where a film formation method such as the coating method or the printing method is employed, a high molecular compound (e.g., an oligomer, a dendrimer, or a polymer), a middle molecular compound (a compound between a low molecular compound and a high molecular compound with a molecular weight of 400 to 4000), an inorganic compound (e.g., a quantum dot material), or the like can be used. The quantum dot material can be a colloidal quantum dot material, an alloyed quantum dot material, a core-shell quantum dot material, a core quantum dot material, or the like.

Materials that can be used for the layers (the hole-injection layer 111, the hole-transport layer 112, the light-emitting layer 113, the electron-transport layer 114, and the electron-injection layer 115) included in the EL layer 103 of the light-emitting device described in this embodiment are not limited to the materials described in this embodiment, and other materials can be used in combination as long as the functions of the layers are fulfilled.

In this specification and the like, the terms “layer” and “film” can be interchanged with each other as appropriate.

The structures described in this embodiment can be used in combination with any of the structures described in the other embodiments as appropriate.

### Embodiment 4

In this embodiment, specific structure examples of a light-emitting and light-receiving apparatus of one embodiment of the present invention and an example of the manufacturing method will be described.

### Structure Example of Light-Emitting and Light-Receiving Apparatus 700

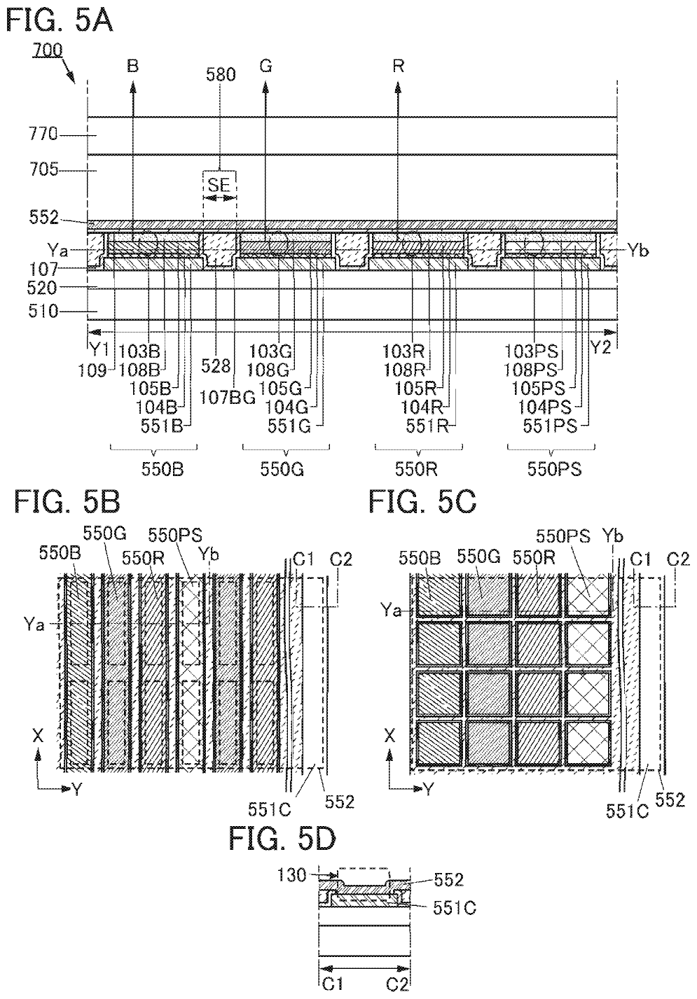

A light-emitting and light-receiving apparatus 700 illustrated in FIG. 5A includes a light-emitting device 550B, a light-emitting device 550G, a light-emitting device 550R, and a light-receiving device 550PS. The light-emitting device 550B, the light-emitting device 550G, the light-emitting device 550R, and the light-receiving device 550PS are formed over a functional layer 520 provided over a first substrate 510. The functional layer 520 includes, for example, circuits such as a circuit GD that are composed of a plurality of transistors, and wirings that electrically connect these circuits. Note that these driver circuits are electrically connected to the light-emitting device 550B, the light-emitting device 550G, the light-emitting device 550R, and the light-receiving device 550PS, for example, to drive them. The light-emitting and light-receiving apparatus 700 includes an insulating layer 705 over the functional layer 520 and the devices (the light-emitting devices and the light-receiving device), and the insulating layer 705 has a function of attaching a second substrate 770 and the functional layer 520.

The light-emitting device 550B, the light-emitting device 550G, the light-emitting device 550R, and the light-receiving device 550PS each have any of the device structures described in Embodiments 2 and 3. Described here is the case where the light-emitting devices have any of the structures illustrated in FIGS. 4A to 4E and the light-receiving device has the structure illustrated in FIG. 1B. Note that the light-emitting and light-receiving apparatus illustrated in FIG. 1B has a structure in which parts of the EL layer (the hole-injection layer, the hole-transport layer, and the electron-transport layer) of the light-emitting device and parts of the active layer (the first transport layer and the second transport layer) of the light-receiving device are concurrently formed using the same material in a manufacturing process; meanwhile, this embodiment describes a case where separation can be made not only between the light-emitting device and the light-receiving device, but also between all the devices (the light-emitting devices and the light-receiving device).

In this specification and the like, a structure in which light-emitting layers in light-emitting devices of different colors (for example, blue (B), green (G), and red (R)) and a light-receiving layer in a light-receiving device are separately formed or separately patterned is sometimes referred to as a side-by-side (SBS) structure. Although the light-emitting device 550B, the light-emitting device 550G, the light-emitting device 550R, and the light-receiving device 550PS are arranged in this order in the light-emitting and light-receiving apparatus 700 illustrated in FIG. 5A, one embodiment of the present invention is not limited to this structure. For example, in the light-emitting and light-receiving apparatus 700, these devices may be arranged in the order of the light-emitting device 550R, the light-emitting device 550G, the light-emitting device 550B, and the light-receiving device 550PS.

In FIG. 5A, the light-emitting device 550B includes an electrode 551B, the electrode 552, and an EL layer 103B. The light-emitting device 550G includes an electrode 551G, the electrode 552, and an EL layer 103G. The light-emitting device 550R includes an electrode 551R, the electrode 552, and an EL layer 103R. The light-receiving device 550PS includes an electrode 551PS, the electrode 552, and a light-receiving layer 103PS. Note that a specific structure of each layer of the light-receiving device is as described in Embodiment 2. In addition, a specific structure of each layer of the light-emitting device is as described in Embodiment 3. The EL layer 103B, the EL layer 103G, and the EL layer 103R each have a stacked-layer structure of layers having different functions including their respective light-emitting layers (105B, 105G, and 105R). The light-receiving layer 103PS has a stacked-layer structure of layers having different functions including an active layer 105PS. FIG. 5A illustrates a case where the EL layer 103B includes a hole-injection/transport layer 104B, a light-emitting layer 105B, an electron-transport layer 108B, and an electron-injection layer 109; the EL layer 103G includes a hole-injection/transport layer 104G, a light-emitting layer 105G, an electron-transport layer 108G, and the electron-injection layer 109; the EL layer 103R includes a hole-injection/transport layer 104R, a light-emitting layer 105R, an electron-transport layer 108R, and the electron-injection layer 109; and the light-receiving layer 103PS includes a first transport layer 104PS, the active layer 105PS, a second transport layer 108PS, and the electron-injection layer 109. However, the present invention is not limited thereto. Note that each of the hole-injection/transport layers (104B, 104G, and 104R) represents a layer having the functions of the hole-injection layer and the hole-transport layer described in Embodiment 3, and may have a stacked-layer structure.

Note that the electron-transport layers (108B, 108G, and 108R) and the second transport layer 108PS may have a function of blocking holes moving from the anode side to the cathode side through the EL layers (103B, 103G, and 103R) and the light-receiving layer 103PS. The electron-injection layer 109 may have a stacked-layer structure in which some or all of layers are formed using different materials.

As illustrated in FIG. 5A, insulating layer 107 may be formed on side surfaces (or end portions) of the hole-injection/transport layers (104B, 104G, and 104R), the light-emitting layers (105B, 105G, and 105R), and the electron-transport layers (108B, 108G, and 108R) included in the EL layers (103B, 103G, and 103R), and side surfaces (or end portions) of the first transport layer 104PS, the active layer 105PS, and the second transport layer 108PS included in the light-receiving layer 103PS. The insulating layer 107 is formed in contact with the side surfaces (or the end portions) of the EL layers (103B, 103G, and 103R) and the light-receiving layer 103PS. This can inhibit entry of oxygen, moisture, or constituent elements thereof into the inside through the side surfaces of the EL layers (103B, 103G, and 103R) and the light-receiving layer 103PS. For the insulating layer 107, aluminum oxide, magnesium oxide, hafnium oxide, gallium oxide, indium gallium zinc oxide, silicon nitride, or silicon nitride oxide can be used, for example. Some of the above-described materials may be stacked to form the insulating layer 107. The insulating layer 107 can be formed by a sputtering method, a CVD method, an MBE method, a PLD method, an ALD method, or the like and is formed preferably by an ALD method, which achieves favorable coverage. Note that the insulating layer 107 continuously covers the side surfaces (or the end portions) of part of the EL layers (103B, 103G, and 103R) and part of the light-receiving layer 103PS of adjacent devices. For example, in FIG. 5A, the side surfaces of part of the EL layer 103B of the light-emitting device 550B and part of the EL layer 103G of the light-emitting device 550G are covered with an insulating layer 107BG. In a region covered with the insulating layer 107BG, a partition wall 528 formed using an insulating material is preferably formed, as illustrated in FIG. 5A.

In addition, the electron-injection layer 109 is formed over the electron-transport layers (108B, 108G, and 108R) that are parts of the EL layers (103B, 103G, and 103R), the second transport layer 108PS that is part of the light-receiving layer 103PS, and the insulating layer 107. Note that the electron-injection layer 109 may have a stacked-layer structure of two or more layers (for example, stacked layers having different electric resistances).

The electrode 552 is formed over the electron-injection layer 109. Note that the electrodes (551i, 551G, and 551R) and the electrode 552 include overlap regions. The light-emitting layer 105B is provided between the electrode 551B and the electrode 552, the light-emitting layer 105G is provided between the electrode 551G and the electrode 552, the light-emitting layer 105R is provided between the electrode 551R and the electrode 552, and the light-receiving layer 103PS is provided between the electrode 551PS and the electrode 552.

The EL layers (103B, 103G, and 103R) illustrated in FIG. 5A each have a structure similar to that of the EL layer 103 described in Embodiment 3. The light-receiving layer 103PS has a structure similar to that of the light-receiving layer 203 described in Embodiment 2. The light-emitting layer 105B can emit blue light, the light-emitting layer 105G can emit green light, and the light-emitting layer 105R can emit red light, for example.

The partition walls 528 and the insulating layer 107 are provided between part of the light-emitting device 550B, part of the light-emitting device 550G, part of the light-emitting device 550R, and part of the light-receiving device 550PS. As illustrated in FIG. 5A, the partition walls 528 are in contact with the side surfaces (or the end portions) of the electrodes (551B, 551G, 551R, and 551PS), parts of the EL layers (103B, 103G, and 103R), and part of the light-receiving layer 103PS with the insulating layer 107 therebetween.

In each of the EL layers and the light-receiving layer, particularly the hole-injection layer, which is included in the hole-transport region between the anode and the light-emitting layer and between the anode and the active layer, often has high conductivity; thus, a hole-injection layer formed as a layer shared by adjacent light-emitting devices or adjacent light-receiving devices might cause crosstalk. Thus, as described in this structure example, the partition walls 528 formed using an insulating material are provided between the EL layers and between the EL layer and the light-receiving layer, which can inhibit occurrence of crosstalk between adjacent devices (between the light-receiving device and the light-emitting device, between the light-emitting devices, or between the light-receiving devices).

In the manufacturing method described in this embodiment, side surfaces (or end portions) of the EL layer and the light-receiving layer are exposed in the patterning step. This may promote deterioration of the EL layer and the light-receiving layer by allowing the entry of oxygen, water, or the like through the side surfaces (or the end portions) of the EL layer and the light-receiving layer. Hence, providing the partition wall 528 can inhibit the deterioration of the EL layer and the light-receiving layer in the manufacturing process.

Providing the partition wall 528 can flatten the surface by reducing a depressed portion formed between adjacent devices (between the light-receiving device and the light-emitting device, between the light-emitting devices, or between the light-receiving devices). When the depressed portion is reduced, disconnection of the electrode 552 formed over the EL layers and the light-receiving layer can be inhibited. Examples of an insulating material used to form the partition wall 528 include organic materials such as an acrylic resin, a polyimide resin, an epoxy resin, an imide resin, a polyamide resin, a polyimide-amide resin, a silicone resin, a siloxane resin, a benzocyclobutene-based resin, a phenol resin, and precursors of these resins. Other examples include organic materials such as polyvinyl alcohol (PVA), polyvinyl butyral, polyvinyl pyrrolidone, polyethylene glycol, polyglycerin, pullulan, water-soluble cellulose, and alcohol-soluble polyamide resin. A photosensitive resin such as a photoresist can also be used. Examples of the photosensitive resin include positive-type materials and negative-type materials.

With the use of the photosensitive resin, the partition wall 528 can be fabricated by only light exposure and developing steps. The partition wall 528 may be fabricated using a negative photosensitive resin (e.g., a resist material). In the case where an insulating layer containing an organic material is used as the partition wall 528, a material absorbing visible light is suitably used. When such a material absorbing visible light is used for the partition wall 528, light emission from the EL layer can be absorbed by the partition wall 528, leading to a reduction in light leakage (stray light) to an adjacent EL layer or light-receiving layer. Accordingly, a display panel with high display quality can be provided.

For example, the difference between the top-surface level of the partition wall 528 and the top-surface level of any of the EL layer 103B, the EL layer 103G, the EL layer 103R, and the light-receiving layer 103PS is preferably 0.5 times or less, further preferably 0.3 times or less the thickness of the partition wall 528. The partition wall 528 may be provided such that the top-surface level of any of the EL layer 103B, the EL layer 103G, the EL layer 103R, and the light-receiving layer 103PS is higher than the top-surface level of the partition wall 528, for example. Alternatively, the partition wall 528 may be provided such that the top-surface level of the partition wall 528 is higher than the top-surface level of any of the EL layer 103B, the EL layer 103G, the EL layer 103R, and the light-receiving layer 103PS, for example.

When electrical continuity is established between the EL layer 103B, the EL layer 103G, the EL layer 103R, and the light-receiving layer 103PS in a light-emitting and light-receiving apparatus (display panel) with a high resolution more than 1000 ppi, crosstalk occurs, resulting in a narrower color gamut that the light-emitting and light-receiving apparatus is capable of reproducing. Providing the partition wall 528 in a high-resolution display panel with more than 1000 ppi, preferably more than 2000 ppi, or further preferably in an ultrahigh-resolution display panel with more than 5000 ppi allows the display panel to express vivid colors.

FIGS. 5B and 5C are each a schematic top view of the light-emitting and light-receiving apparatus 700 taken along the dashed-dotted line Ya-Yb in the cross-sectional view of FIG. 5A. Specifically, the light-emitting device 550B, the light-emitting device 550G, and the light-emitting device 550R are arranged in a matrix. Note that FIG. 5B illustrates what is called a stripe arrangement, in which the light-emitting devices of the same color are arranged in the X-direction. FIG. 5C illustrates a structure in which the light-emitting devices of the same color are arranged in the X-direction and separated by patterning for each pixel. Note that the arrangement method of the light-emitting devices is not limited thereto; another method such as a delta, zigzag, PenTile, or diamond arrangement may also be used.

The EL layers (the EL layer 103B, the EL layer 103G, and the EL layer 103R) and the light-receiving layer 103PS are processed to be separated by patterning using a photolithography method; hence, a light-emitting and light-receiving apparatus (display panel) with a high resolution can be fabricated. The end portions (side surfaces) of the EL layer and the light-receiving layer 103PS processed by patterning using a photolithography method have substantially the same surface (or are positioned on substantially the same plane). In this case, the widths (SE) of spaces 580 between the EL layers and between the EL layer and the light-receiving layer are each preferably 5 m or less, further preferably 1 μm or less.

In the EL layer, particularly the hole-injection layer, which is included in the hole-transport region between the anode and the light-emitting layer, often has high conductivity; thus, a hole-injection layer formed as a layer shared by adjacent light-emitting devices might cause crosstalk. Therefore, processing the EL layers to be separated by patterning using a photolithography method as described in this structure example can suppress occurrence of crosstalk between adjacent light-emitting devices.

FIG. 5D is a schematic cross-sectional view taken along the dashed-dotted line C1-C2 in FIGS. 5B and 5C. FIG. 5D illustrates a connection portion 130 where a connection electrode 551C and the electrode 552 are electrically connected to each other. In the connection portion 130, the electrode 552 is provided over and in contact with the connection electrode 551C. The partition wall 528 is provided to cover an end portion of the connection electrode 551C.

### Example of Method for Manufacturing Light-Emitting and Light-Receiving Apparatus

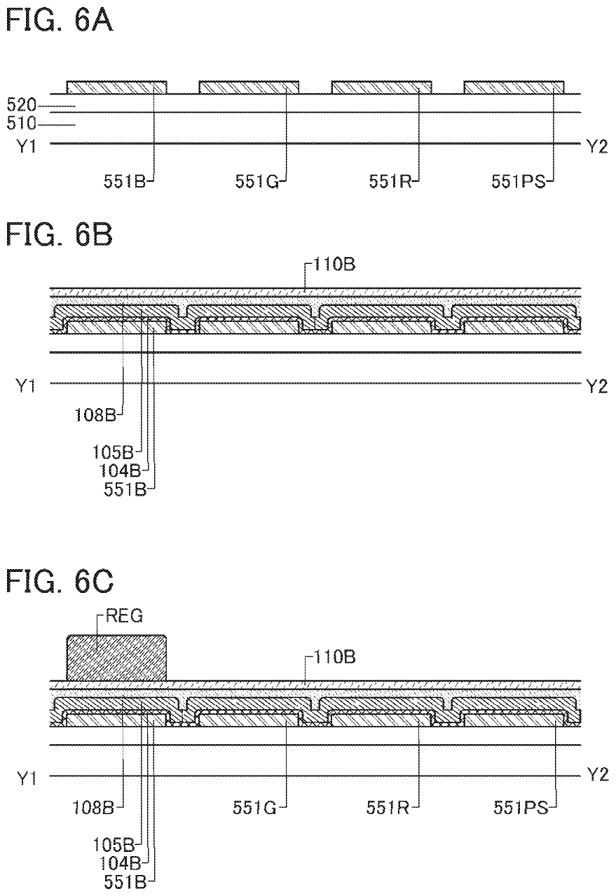

The electrode 551B, the electrode 551G, the electrode 551R, and the electrode 551PS are formed as illustrated in FIG. 6A. For example, a conductive film is formed over the functional layer 520 over the first substrate 510 and processed into predetermined shapes by a photolithography method.

The conductive film can be formed by any of a sputtering method, a chemical vapor deposition (CVD) method, a molecular beam epitaxy (MBE) method, a vacuum evaporation method, a pulsed laser deposition (PLD) method, an atomic layer deposition (ALD) method, and the like. Examples of the CVD method include a plasma-enhanced chemical vapor deposition (PECVD) method and a thermal CVD method. An example of a thermal CVD method is a metal organic CVD (MOCVD) method.

The conductive film may be processed by a nanoimprinting method, a sandblasting method, a lift-off method, or the like as well as a photolithography method described above. Alternatively, island-shaped thin films may be directly formed by a film formation method using a shielding mask such as a metal mask. Here, “island shape” refers to a state where layers formed using the same material in the same step are separated from each other when seen from above.

There are two typical examples of photolithography methods. In one of the methods, a resist mask is formed over a thin film that is to be processed, the thin film is processed by etching or the like, and then the resist mask is removed. In the other method, a photosensitive thin film is formed and then processed into a desired shape by light exposure and development. The former method involves heat treatment steps such as pre-applied bake (PAB) after resist application and post-exposure bake (PEB) after light exposure. In one embodiment of the present invention, a lithography method is used not only for processing of a conductive film but also for processing of a thin film used for formation of an EL layer (a film made of an organic compound or a film partly including an organic compound).

As light for exposure in a photolithography method, it is possible to use light with the i-line (wavelength: 365 nm), light with the g-line (wavelength: 436 nm), light with the h-line (wavelength: 405 nm), or light in which the i-line, the g-line, and the h-line are mixed. Alternatively, ultraviolet light, KrF laser light, ArF laser light, or the like can be used. Exposure may be performed by liquid immersion exposure technique. As the light for exposure, extreme ultraviolet (EUV) light or X-rays may also be used. Instead of the light for exposure, an electron beam can be used. It is preferable to use EUV, X-rays, or an electron beam because extremely minute processing can be performed. Note that a photomask is not needed when light exposure is performed by scanning with a beam such as an electron beam.

For etching of a thin film using a resist mask, a dry etching method, a wet etching method, a sandblast method, or the like can be used.

Subsequently, as illustrated in FIG. 6B, the hole-injection/transport layer 104B, the light-emitting layer 105B, and the electron-transport layer 108B are formed over the electrode 551B, the electrode 551G, the electrode 551R, and the electrode 551PS. Note that the hole-injection/transport layer 104B, the light-emitting layer 105B, and the electron-transport layer 108B can be formed using a vacuum evaporation method, for example. Furthermore, a sacrifice layer 110 is formed over the electron-transport layer 108B. For the formation of the hole-injection/transport layer 104B, the light-emitting layer 105B, and the electron-transport layer 108B, any of the materials described in Embodiment 3 can be used.

For the sacrifice layer 110B, it is preferable to use a film highly resistant to etching treatment performed on the hole-injection/transport layer 104B, the light-emitting layer 105B, and the electron-transport layer 108B, i.e., a film having high etching selectivity with respective to the hole-injection/transport layer 104B, the light-emitting layer 105B, and the electron-transport layer 108B. The sacrifice layer 110B preferably has a stacked-layer structure of a first sacrifice layer and a second sacrifice layer which have different etching selectivities. For the sacrifice layer 110B, it is possible to use a film that can be removed by a wet etching method, which causes less damage to the EL layer 103B. In wet etching, oxalic acid or the like can be used as an etching material.

For the sacrifice layer 110B, an inorganic film such as a metal film, an alloy film, a metal oxide film, a semiconductor film, or an inorganic insulating film can be used, for example. The sacrifice layer 110B can be formed by any of a variety of film formation methods such as a sputtering method, an evaporation method, a CVD method, and an ALD method.

For the sacrifice layer 110B, a metal material such as gold, silver, platinum, magnesium, nickel, tungsten, chromium, molybdenum, iron, cobalt, copper, palladium, titanium, aluminum, yttrium, zirconium, or tantalum or an alloy material containing the metal material can be used, for example. It is particularly preferable to use a low-melting-point material such as aluminum or silver.

A metal oxide such as indium gallium zinc oxide (also referred to as In—Ga—Zn oxide or IGZO) can be used for the sacrifice layer 110B. It is also possible to use indium oxide, indium zinc oxide (In—Zn oxide), indium tin oxide (In—Sn oxide), indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or the like. Alternatively, indium tin oxide containing silicon can also be used, for example.

An element M(M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used instead of gallium. In particular, M is preferably one or more of gallium, aluminum, and yttrium.

For the sacrifice layer 110B, an inorganic insulating material such as aluminum oxide, hafnium oxide, or silicon oxide can be used.

The sacrifice layer 110B is preferably formed using a material that can be dissolved in a solvent chemically stable with respect to at least the electron-transport layer 108B that is in the uppermost position. Specifically, a material that can be dissolved in water or alcohol can be suitably used for the sacrifice layer 110B. In formation of the sacrifice layer 110B, it is preferable that application of such a material dissolved in a solvent such as water or alcohol be performed by a wet process and followed by heat treatment for evaporating the solvent. At this time, the heat treatment is preferably performed under a reduced-pressure atmosphere, in which case the solvent can be removed at a low temperature in a short time and thermal damage to the hole-injection/transport layer 104B, the light-emitting layer 105B, and the electron-transport layer 108B can be accordingly reduced.

In the case where the sacrifice layer 110B having a stacked-layer structure is formed, the stacked-layer structure can include the first sacrifice layer formed using any of the above-described materials and the second sacrifice layer thereover.

The second sacrifice layer in that case is a film used as a hard mask for etching of the first sacrifice layer. In processing the second sacrifice layer, the first sacrifice layer is exposed. Thus, a combination of films having greatly different etching rates is selected for the first sacrifice layer and the second sacrifice layer. Thus, a film that can be used for the second sacrifice layer can be selected in accordance with the etching conditions of the first sacrifice layer and those of the second sacrifice layer.

For example, in the case where the second sacrifice layer is etched by dry etching involving a fluorine-containing gas (also referred to as a fluorine-based gas), the second sacrifice layer can be formed using silicon, silicon nitride, silicon oxide, tungsten, titanium, molybdenum, tantalum, tantalum nitride, an alloy containing molybdenum and niobium, an alloy containing molybdenum and tungsten, or the like. Here, a film of a metal oxide such as IGZO or ITO can be given as an example of a film having a high etching selectivity to the second sacrifice layer (i.e., a film with a low etching rate) in the dry etching involving the fluorine-based gas, and can be used for the first sacrifice layer.

Note that the material for the second sacrifice layer is not limited to the above and can be selected from a variety of materials in accordance with the etching conditions of the first sacrifice layer and those of the second sacrifice layer. For example, any of the films that can be used for the first sacrifice layer can be used for the second sacrifice layer.

For the second sacrifice layer, a nitride film can be used, for example. Specifically, it is possible to use a nitride such as silicon nitride, aluminum nitride, hafnium nitride, titanium nitride, tantalum nitride, tungsten nitride, gallium nitride, or germanium nitride.

Alternatively, an oxide film can be used for the second sacrifice layer. Typically, it is possible to use a film of an oxide or an oxynitride such as silicon oxide, silicon oxynitride, aluminum oxide, aluminum oxynitride, hafnium oxide, or hafnium oxynitride.

Next, as illustrated in FIG. 6C, a resist is applied onto the sacrifice layer 110B, and the resist having a desired shape (a resist mask REG) is formed by a photolithography method. Such a method involves heat treatment steps such as pre-applied bake (PAB) after the resist application and post-exposure bake (PEB) after light exposure. The temperature reaches approximately 100° C. during the PAB, and approximately 120° C. during the PEB, for example. Therefore, the light-emitting device should be resistant to such high treatment temperatures.

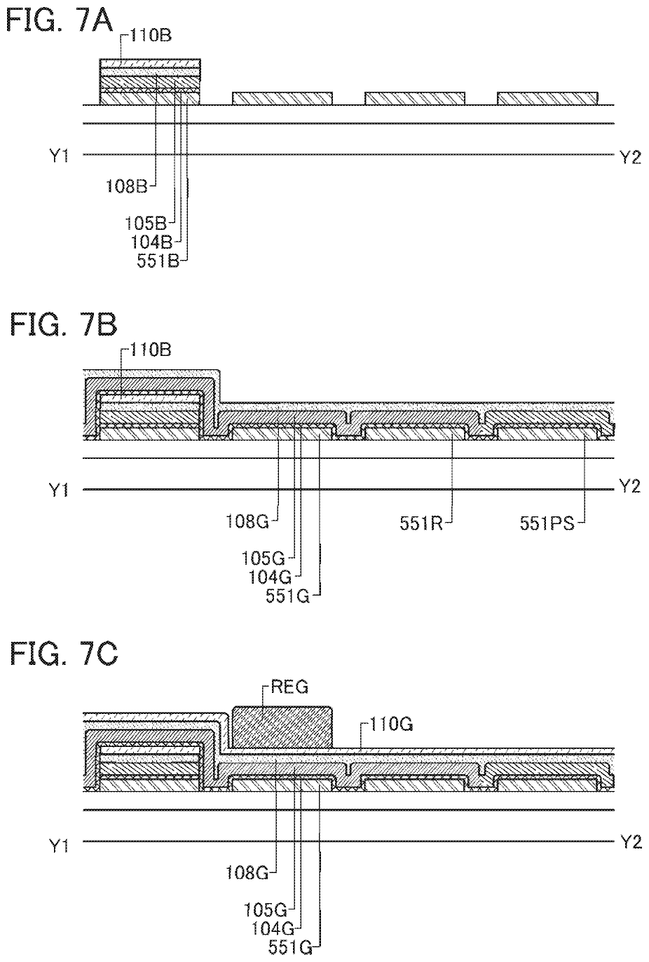

Next, part of the sacrifice layer 110B that is not covered with the resist mask REG is removed by etching using the resist mask REG, the resist mask REG is removed, and then the hole-injection/transport layer 104B, the light-emitting layer 105B, and the electron-transport layer 108B that are not covered with the sacrifice layer are removed by etching, so that the hole-injection/transport layer 104B, the light-emitting layer 105B, and the electron-transport layer 108B are processed to have side surfaces (or have their side surfaces exposed) over the electrode 551B or have belt-like shapes extending in the direction intersecting the sheet of the diagram. Note that dry etching is preferably employed for the etching. Note that in the case where the sacrifice layer 110B has the aforementioned stacked-layer structure of the first sacrifice layer and the second sacrifice layer, the hole-injection/transport layer 104B, the light-emitting layer 105B, and the electron-transport layer 108B may be processed into a predetermined shape in the following manner: part of the second sacrifice layer is etched using the resist mask REG, the resist mask REG is then removed, and part of the first sacrifice layer is etched using the second sacrifice layer as a mask. The structure illustrated in FIG. 7A is obtained through these etching steps.

Subsequently, as illustrated in FIG. 7B, the hole-injection/transport layer 104G, the light-emitting layer 105G, and the electron-transport layer 108G are formed over the sacrifice layer 110B, the electrode 551G, the electrode 551R, and the electrode 551PS. The hole-injection/transport layer 104G, the light-emitting layer 105G, and the electron-transport layer 108G can be formed using any of the materials described in Embodiment 3. Note that the hole-injection/transport layer 104G, the light-emitting layer 105G, and the electron-transport layer 108G can be formed by a vacuum evaporation method, for example.

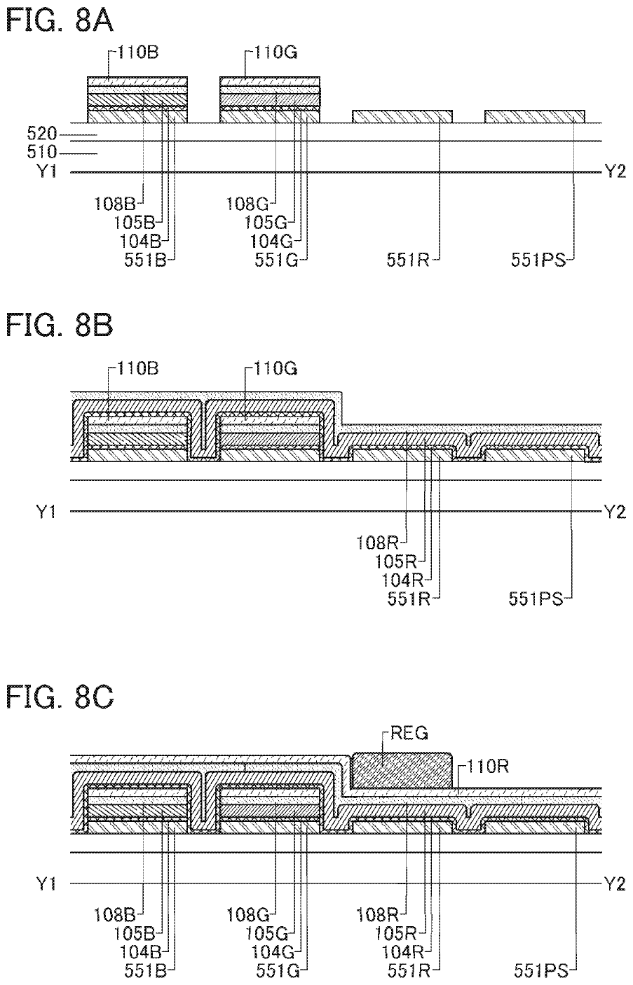

Next, as illustrated in FIG. 7C, the sacrifice layer 110G is formed over the electron-transport layer 108G, a resist is applied onto the sacrifice layer 110G, and the resist having a desired shape (the resist mask REG) is formed by a lithography method. Part of the sacrifice layer that is not covered with the obtained resist mask is removed by etching, the resist mask is removed, and then parts of the hole-injection/transport layer 104G, the light-emitting layer 105G, and the electron-transport layer 108G that are not covered with the sacrifice layer are removed by etching. Thus, the hole-injection/transport layer 104G, the light-emitting layer 105G, and the electron-transport layer 108G are processed to have side surfaces (or have their side surfaces exposed) over the electrode 551G or have belt-like shapes extending in the direction intersecting the sheet of the diagram. Note that dry etching is preferably employed for the etching. Note that the sacrifice layer 110G can be formed using a material similar to that for the sacrifice layer 110B. In the case where the sacrifice layer 110G has the aforementioned stacked-layer structure of the first sacrifice layer and the second sacrifice layer, the hole-injection/transport layer 104G, the light-emitting layer 105G, and the electron-transport layer 108G may be processed into a predetermined shape in the following manner: part of the second sacrifice layer is etched using the resist mask, the resist mask is then removed, and part of the first sacrifice layer is etched using the second sacrifice layer as a mask. The structure illustrated in FIG. 8A is obtained through these etching steps.

Next, as illustrated in FIG. 8B, the hole-injection/transport layer 104R, the light-emitting layer 105R, and the electron-transport layer 108R are formed over the sacrifice layer 110B, the sacrifice layer 110G, the electrode 551R, and the electrode 551PS. The hole-injection/transport layer 104R, the light-emitting layer 105R, and the electron-transport layer 108R can be formed using any of the materials described in Embodiment 3. The hole-injection/transport layer 104R, the light-emitting layer 105R, and the electron-transport layer 108R can be formed by a vacuum evaporation method, for example.

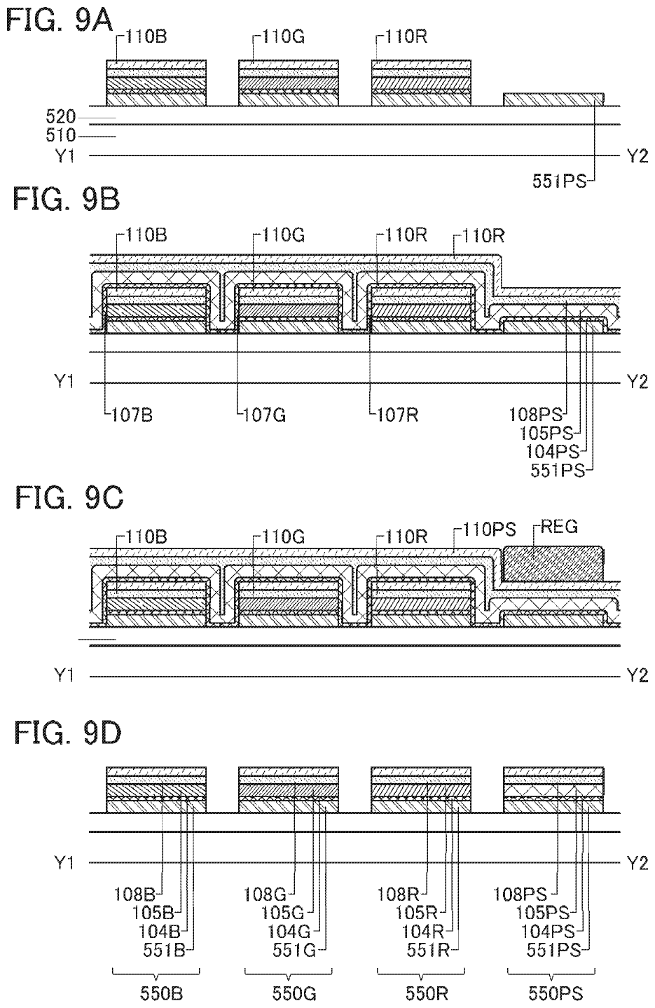

Next, as illustrated in FIG. 8C, the sacrifice layer 110R is formed over the electron-transport layer 108R, a resist is applied onto the sacrifice layer 110R, and the resist having a desired shape (the resist mask REG) is formed by a photolithography method. Part of the sacrifice layer 110R that is not covered with the obtained resist mask is removed by etching, the resist mask is removed, and then the hole-injection/transport layer 104R, the light-emitting layer 105R, and the electron-transport layer 108R that are not covered with the sacrifice layer are removed by etching. Thus, the hole-injection/transport layer 104R, the light-emitting layer 105R, and the electron-transport layer 108R are processed to have side surfaces (or have their side surfaces exposed) over the electrode 551R or have belt-like shapes extending in the direction intersecting the sheet of the diagram. Note that dry etching is preferably employed for the etching. Note that the sacrifice layer 110R can be formed using a material similar to that for the sacrifice layer 110B. In the case where the sacrifice layer 110R has the aforementioned stacked-layer structure of the first sacrifice layer and the second sacrifice layer, the hole-injection/transport layer 104R, the light-emitting layer 105R, and the electron-transport layer 108R may be processed into a predetermined shape in the following manner: part of the second sacrifice layer is etched using the resist mask, the resist mask is then removed, and part of the first sacrifice layer is etched using the second sacrifice layer as a mask. The structure illustrated in FIG. 9A is obtained through these etching steps.

Next, as illustrated in FIG. 9B, the first transport layer 104PS, the active layer 105PS, and the second transport layer 108PS are formed over the sacrifice layer 110B, the sacrifice layer 110G, the sacrifice layer 110R, and the electrode 551PS. The first transport layer 104PS, the active layer 105PS, and the second transport layer 108PS can be formed using any of the materials described in Embodiment 2. Note that the first transport layer 104PS, the active layer 105PS, and the second transport layer 108PS can be formed by a vacuum evaporation method, for example.

Next, as illustrated in FIG. 9C, the sacrifice layer 110PS is formed over the second transport layer 108PS, a resist is applied onto the sacrifice layer 110PS, and the resist having a desired shape (the resist mask REG) is formed by a photolithography method. Part of the sacrifice layer 110PS that is not covered with the obtained resist mask is removed by etching, the resist mask is removed, and then parts of the first transport layer 104PS, the active layer 105PS, and the second transport layer 108PS that are not covered with the sacrifice layer 110PS are removed by etching. Thus, the first transport layer 104PS, the active layer 105PS, and the second transport layer 108PS are processed to have side surfaces (or have their side surfaces exposed) over the electrode 551PS or have belt-like shapes extending in the direction intersecting the sheet of the diagram. Note that dry etching is preferably employed for the etching. Note that the sacrifice layer 110PS can be formed using a material similar to that for the sacrifice layer 110B. In the case where the sacrifice layer 110PS has the aforementioned stacked-layer structure of the first sacrifice layer and the second sacrifice layer, the first transport layer 104PS, the active layer 105PS, and the second transport layer 108PS may be processed into a predetermined shape in the following manner: part of the second sacrifice layer is etched using the resist mask, the resist mask is then removed, and part of the first sacrifice layer is etched using the second sacrifice layer as a mask. The structure illustrated in FIG. 9D is obtained through these etching steps.

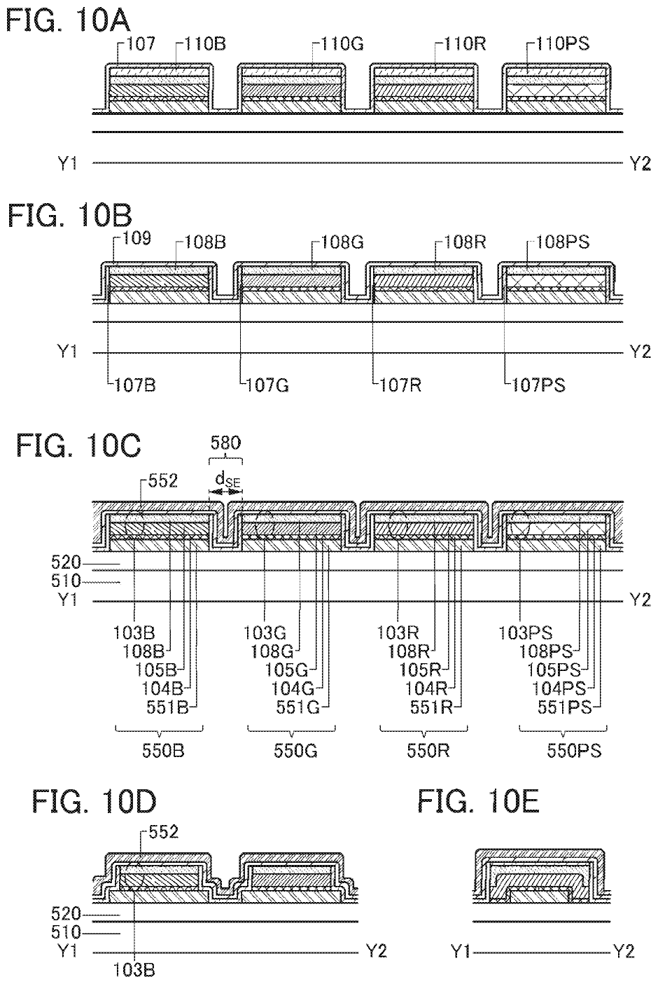

Next, as illustrated in FIG. 10A, the insulating layer 107 is formed over the sacrifice layer 110B, the sacrifice layer 110G, the sacrifice layer 110R, and the sacrifice layer 110PS.

Note that the insulating layer 107 can be formed by an ALD method, for example. In this case, as illustrated in FIG. 10A, the insulating layer 107 is formed to be in contact with the side surfaces (end portions) of the hole-injection/transport layers (104B, 104G, and 104R), the light-emitting layers (105R, 105G, and 105B), and the electron-transport layers (108B, 108G, and 108R) of the light-emitting devices and the first transport layer 104PS, the light-receiving layer 103PS, and the second transport layer 108PS of the light-receiving device. This can inhibit entry of oxygen, moisture, or constituent elements thereof into the inside through the side surfaces of the layers. Examples of the material used for the insulating layer 107 include aluminum oxide, magnesium oxide, hafnium oxide, gallium oxide, indium gallium zinc oxide, silicon nitride, and silicon nitride oxide.

Next, as illustrated in FIG. 10B, the sacrifice layers (110B, 110G, 110R, and 110PS) are removed, and then, the electron-injection layer 109 is formed over the insulating layers (107B, 107G, 107R, and 107PS), the electron-transport layers (108B, 108G, and 108R), and the second transport layer 108PS. The electron-injection layer 109 can be formed using any of the materials described in Embodiment 3. The electron-injection layer 109 is formed by a vacuum evaporation method, for example. The electron-injection layer 109 is formed over the electron-transport layers (108B, 108G, and 108R) and the second transport layer 108PS. The electron-injection layer 109 is in contact with the side surfaces (end portions) of the hole-injection/transport layers (104B, 104G, and 104R), the light-emitting layers (105R, 105G, and 105B), and the electron-transport layers (108B, 108G, and 108R) of the light-emitting devices and the first transport layer 104PS, the active layer 105PS, and the second transport layer 108PS of the light-receiving device with the insulating layers (107B, 107G, 107R, and 107PS) therebetween.

Next, as illustrated in FIG. 10C, the electrode 552 is formed. The electrode 552 is formed by a vacuum evaporation method, for example. The electrode 552 is formed over the electron-injection layer 109. Note that the electrode 552 is in contact with the side surfaces (end portions) of the hole-injection/transport layers (104B, 104G, and 104R), the light-emitting layers (105R, 105G, and 105B), and the electron-transport layers (108B, 108G, and 108R) of the light-emitting devices and the first transport layer 104PS, the active layer 105PS, and the second transport layer 108PS of the light-receiving device with the electron-injection layer 109 and the insulating layers (107B, 107G, 107R, and 107PS) therebetween. This can prevent electrical short circuits between the electrode 552 and each of the following layers: the hole-injection/transport layers (104B, 104G, and 104R), the light-emitting layers (105R, 105G, and 105B), and the electron-transport layers (108B, 108G, and 108R) of the light-emitting devices and the first transport layer 104PS, the active layer 105PS, and the second transport layer 108PS of the light-receiving layer.

Through the above steps, the EL layer 103B, the EL layer 103G, the EL layer 103R, and the light-receiving layer 103PS in the light-emitting device 550B, the light-emitting device 550G, the light-emitting device 550R, and the light-receiving device 550PS can be processed to be separated from each other.

The EL layers (the EL layer 103B, the EL layer 103G, and the EL layer 103R) and the light-receiving layer 103PS are processed to be separated by patterning using a photolithography method; hence, a light-emitting and light-receiving apparatus (display panel) with a high resolution can be fabricated. End portions (side surfaces) of the EL layer and the light-receiving layer 103PS processed by patterning using a photolithography method have substantially the same surface (or are positioned on substantially the same plane).

Each of the hole-injection/transport layers (104B, 104G, and 104R) of the EL layers and the first transport layer 104PS of the light-receiving layer often has high conductivity, and thus might cause crosstalk when formed as a layer shared by adjacent light-emitting devices. Therefore, processing the EL layers to be separated by patterning using a photolithography method as described in this structure example can inhibit occurrence of crosstalk between adjacent light-emitting devices and light-receiving devices.

In this structure example, the hole-injection/transport layers (104B, 104G, and 104R), the light-emitting layers (105R, 105G, and 105B), and the electron-transport layers (108B, 108G, and 108R) of the EL layers (the EL layer 103B, the EL layer 103G, and the EL layer 103R) included in the light-emitting devices and the first transport layer 104PS, the active layer 105PS, and the second transport layer 108PS of the light-receiving layer 103PS included in the light-receiving device are processed to be separated by patterning using a photolithography method; thus, the end portions (side surfaces) of the processed EL layer and light-receiving layer have substantially the same surface (or are positioned on substantially the same plane).

In addition, the hole-injection/transport layers (104B, 104G, and 104R), the light-emitting layers (105R, 105G, and 105B), and the electron-transport layers (108B, 108G, and 108R) of the EL layers (the EL layer 103B, the EL layer 103G, and the EL layer 103R) included in the light-emitting devices and the first transport layer 104PS, the active layer 105PS, and the second transport layer 108PS of the light-receiving layer 103PS included in the light-receiving device are processed to be separated by patterning using a photolithography method. Thus, the space 580 is provided between the processed end portions (side surfaces) of adjacent devices. In FIG. 10C, when the space 580 is denoted by a distance SE, decreasing the distance d_{SE} increases the aperture ratio and the resolution. By contrast, as the distance d_{SE} is increased, the effect of the difference in the fabrication process between the adjacent devices becomes permissible, which leads to an increase in manufacturing yield. Since the light-emitting device and light-receiving device fabricated according to this specification are suitable for a miniaturization process, the distance d_{SE} between the EL layers or between the EL layer and the light-receiving layer of adjacent devices can be longer than or equal to 0.5 μm and shorter than or equal to 5 μm, preferably longer than or equal to 1 μm and shorter than or equal to 3 μm, further preferably longer than or equal to 1 μm and shorter than or equal to 2.5 μm, and still further preferably longer than or equal to 1 μm and shorter than or equal to 2 m. Typically, the distance d_{SE} is preferably longer than or equal to 1 μm and shorter than or equal to 2 μm (e.g., 1.5 μm or a neighborhood thereof).

In this specification and the like, a device formed using a metal mask or a fine metal mask (FMM) is sometimes referred to as a device having a metal mask (MM) structure. In this specification and the like, a device formed without using a metal mask or an FMM is sometimes referred to as a device having a metal maskless (MML) structure.

Note that the island-shaped EL layers of the light-emitting and light-receiving apparatus having the MML structure are formed by not patterning using a metal mask but processing after formation of an EL layer. Thus, a light-emitting and light-receiving apparatus with a higher resolution or a higher aperture ratio than a conventional one can be achieved. Moreover, EL layers can be formed separately for each color, which enables extremely clear images; thus, a light-emitting and light-receiving apparatus with a high contrast and high display quality can be achieved. Furthermore, provision of a sacrifice layer over an EL layer can reduce damage on the EL layer during the manufacturing process and increase the reliability of the light-emitting device.

In FIG. 5A and FIG. 10C, the widths of the EL layers (103B, 103G, and 103R) are substantially equal to those of the electrodes (551i, 551G, and 551R) in the light-emitting device 550B, the light-emitting device 550G, and the light-emitting device 550R, and the width of the light-receiving layer 103PS is substantially equal to that of the electrode 551PS in the light-receiving device 550PS; however, one embodiment of the present invention is not limited thereto.

In the light-emitting device 550B, the light-emitting device 550G, and the light-emitting device 550R, the widths of the EL layers (103B, 103G, and 103R) may be smaller than those of the electrodes (551i, 551G, and 551R). In the light-receiving device 550PS, the width of the light-receiving layer 103PS may be smaller than that of the electrode 551PS. FIG. 10D illustrates an example in which the widths of the EL layers (103B and 103G) are smaller than those of the electrodes (551B and 551G) in the light-emitting device 550B and the light-emitting device 550G.

In the light-emitting device 550B, the light-emitting device 550G, and the light-emitting device 550R, the widths of the EL layers (103B, 103G, and 103R) may be larger than those of the electrodes (551i, 551G, and 551R). In the light-receiving device 550PS, the width of the light-receiving layer 103PS may be larger than that of the electrode 551PS. FIG. 10E illustrates an example in which the width of the EL layer 103R is larger than that of the electrode 551R in the light-emitting device 550R.

In processing part of the EL layer into an island shape, the stacked-layer structure in which components up to the light-emitting layer are formed can be processed by a photolithography method. In that case, damage to the light-emitting layer (e.g., processing damage) might significantly degrade the reliability. In view of the above, in the manufacture of the display panel of one embodiment of the present invention, a mask layer or the like is preferably formed over a layer above the light-emitting layer (e.g., a carrier-transport layer or a carrier-injection layer, and specifically an electron-transport layer or an electron-injection layer), followed by the processing of the light-emitting layer into an island shape. Such a method provides a highly reliable display panel.

For example, an island-shaped light-emitting layer can be formed by a vacuum evaporation method using a metal mask. However, this method causes a deviation from the designed shape and position of an island-shaped light-emitting layer due to various influences such as the low accuracy of the metal mask position, the positional deviation between the metal mask and a substrate, a warp of the metal mask, and the vapor-scattering-induced expansion of outline of the formed film; accordingly, it is difficult to achieve high resolution and high aperture ratio of the display apparatus. In addition, the outline of a layer may blur during vapor deposition, whereby the thickness of its end portion may be small. That is, the thickness of an island-shaped light-emitting layer may vary from area to area. In the case of manufacturing a display apparatus with a large size, high definition, or high resolution, the manufacturing yield might be reduced because of low dimensional accuracy of the metal mask and deformation due to heat or the like.

In view of the above, in manufacture of the display apparatus of one embodiment of the present invention, a light-emitting layer is formed across a plurality of pixel electrodes that have been formed independently for respective subpixels. After that, the light-emitting layer is processed by a photolithography method for example, so that one island-shaped light-emitting layer is formed per pixel electrode. Thus, the light-emitting layer can be divided into island-shaped light-emitting layers for respective subpixels.

In a possible way of processing the light-emitting layer into an island shape, the light-emitting layer is processed directly by a photolithography method. In that case, damage to the light-emitting layer (e.g., processing damage) might significantly degrade the reliability. In view of the above, in the manufacture of the display apparatus of one embodiment of the present invention, a mask layer (also referred to as a sacrificial layer or a protective layer, for example), or the like is preferably formed over a layer above the light-emitting layer (e.g., a carrier-transport layer or a carrier-injection layer, and specifically an electron-transport layer or an electron-injection layer), followed by the processing of the light-emitting layer into an island shape. Such a method provides a highly reliable display apparatus.

In the above manner, in the method for manufacturing the display device of one embodiment of the present invention, the island-shaped light-emitting layer is formed by processing a light-emitting layer formed on the entire surface, not by using a fine metal mask. Specifically, the size of the island-shaped light-emitting layer is obtained by division and scale down of the light-emitting layer by a photolithography method or the like. Thus, its size can be made smaller than the size of the light-emitting layer formed using a fine metal mask. Accordingly, a high-resolution display apparatus or a display apparatus with a high aperture ratio, which has been difficult to be formed so far, can be achieved.

The small number of times of processing of the light-emitting layer with a photolithography method is preferable because a reduction in manufacturing cost and an improvement of manufacturing yield become possible.

A formation method using a fine metal mask, for example, does not easily reduce the distance between adjacent light-emitting devices to less than 10 m. However, the method using a photolithography method according to one embodiment of the present invention can shorten the distance between adjacent light-emitting devices to less than 10 μm, 5 μm or less, 3 μm or less, 2 μm or less, 1.5 μm or less, 1 μm or less, or even 0.5 μm or less, for example, in a process over a glass substrate. Using a light exposure apparatus for LSI can further shorten the distance between adjacent light-emitting devices to 500 nm or less, 200 nm or less, 100 nm or less, or even 50 nm or less, for example, in a process over a Si wafer. Accordingly, the area of a non-light-emitting region that may exist between two light-emitting devices can be significantly reduced, and the aperture ratio can be close to 100%. For example, the display apparatus of one embodiment of the present invention can achieve an aperture ratio higher than or equal to 40%, higher than or equal to 50%, higher than or equal to 60%, higher than or equal to 70%, higher than or equal to 80%, or higher than or equal to 90%; that is, an aperture ratio lower than 100%.

Increasing the aperture ratio of the display apparatus can improve the reliability of the display apparatus. Specifically, with reference to the lifetime of a display apparatus including an organic EL device and having an aperture ratio of 10%, a display apparatus having an aperture ratio of 20% (that is, two times the aperture ratio of the reference) has a lifetime that is 3.25 times as long as the that of the reference, and a display apparatus having an aperture ratio of 40% (that is, four times the aperture ratio of the reference) has a lifetime that is 10.6 times as long as that of the reference. Thus, the density of current flowing to the organic EL device can be reduced with increasing aperture ratio, and accordingly the lifetime of the display apparatus can be increased. The display device of one embodiment of the present invention can have a higher aperture ratio and thus can have higher display quality. Furthermore, the display apparatus of one embodiment of the present invention has excellent effect that the reliability (especially the lifetime) can be significantly improved with increasing aperture ratio.

The structures described in this embodiment can be used in combination with any of the structures described in the other embodiments as appropriate.

### Embodiment 5

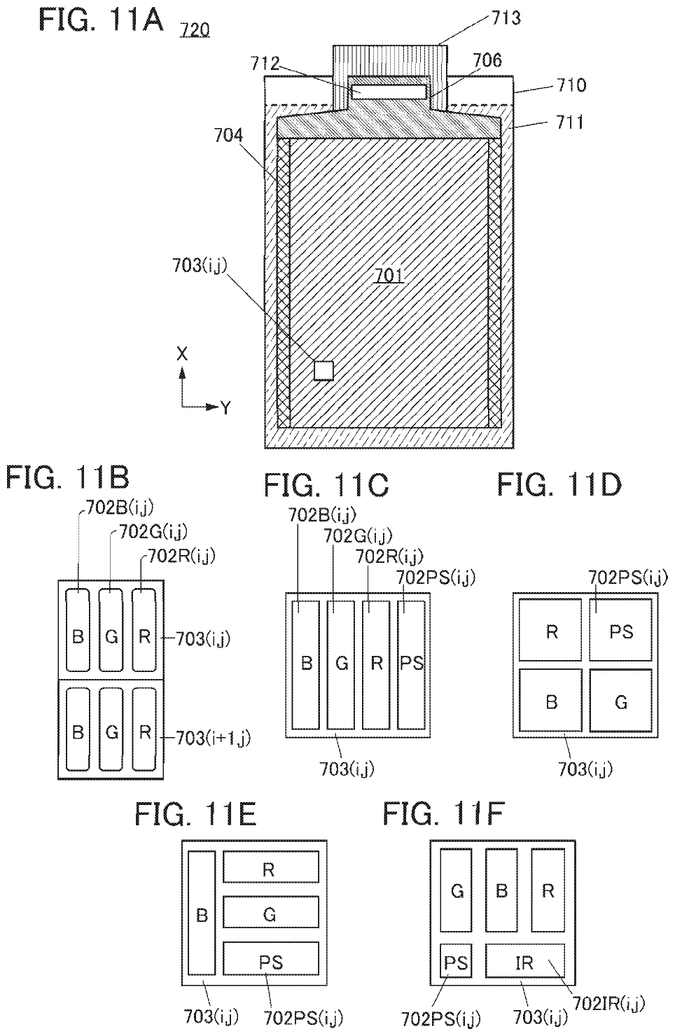

In this embodiment, a light-emitting and light-receiving apparatus 720 is described with reference to FIGS. 11A to 11F, FIGS. 12A to 12C, and FIG. 13. The light-emitting and light-receiving apparatus 720 illustrated in FIGS. 11A to 11F, FIGS. 12A to 12C, and FIG. 13 includes any of the light-receiving devices and the light-emitting devices described in Embodiments 2 and 3 and therefore is a light-emitting and light-receiving apparatus. Furthermore, the light-emitting and light-receiving apparatus 720 described in this embodiment can be used in a display portion of an electronic appliance or the like and therefore can also be referred to as a display panel or a display apparatus. Moreover, the light-emitting and light-receiving apparatus has a structure in which the light-emitting device is used as a light source and the light-receiving device receives light from the light-emitting device.

Furthermore, the light-emitting and light-receiving apparatus of this embodiment can have high definition or large size. Therefore, the light-emitting and light-receiving apparatus of this embodiment can be used, for example, in display portions of electronic appliances such as a digital camera, a digital video camera, a digital photo frame, a mobile phone, a portable game machine, a smart phone, a wristwatch terminal, a tablet terminal, a portable information terminal, and an audio reproducing apparatus, in addition to display portions of electronic appliances with a relatively large screen, such as a television apparatus, a desktop or laptop personal computer, a monitor of a computer or the like, digital signage, and a large game machine such as a pachinko machine.

FIG. 11A is a top view of the light-emitting and light-receiving apparatus 720.

In FIG. 11A, the light-emitting and light-receiving apparatus 720 has a structure in which a substrate 710 and a substrate 711 are attached to each other. In addition, the light-emitting and light-receiving apparatus 720 includes a display region 701, a circuit 704, a wiring 706, and the like. Note that the display region 701 includes a plurality of pixels. As illustrated in FIG. 11B, a pixel 703(i, j) illustrated in FIG. 11A and a pixel 703(i+1, j) are adjacent to each other.

Furthermore, in the example of the light-emitting and light-receiving apparatus 720 illustrated in FIG. 11A, the substrate 710 is provided with an integrated circuit (IC) 712 by a chip on glass (COG) method, a chip on film (COF) method, or the like. As the IC 712, an IC including a scan line driver circuit, a signal line driver circuit, or the like can be used, for example. In the example illustrated in FIG. 11A, an IC including a signal line driver circuit is used as the IC 712, and a scan line driver circuit is used as the circuit 704.

The wiring 706 has a function of supplying signals and power to the display region 701 and the circuit 704. The signals and power are input to the wiring 706 from the outside through a flexible printed circuit (FPC) 713 or to the wiring 706 from the IC 712. Note that the light-emitting and light-receiving apparatus 720 is not necessarily provided with the IC. The IC may be mounted on the FPC by a COF method or the like.

FIG. 11B illustrates the pixel 703(i, j) and the pixel 703(i+1, j) of the display region 701. A plurality of kinds of subpixels including light-emitting devices that emit different color light from each other can be included in the pixel 703(i,j). Alternatively, a plurality of subpixels including light-emitting devices that emit the same color light may be included in addition to those described above. For example, the pixel can include three kinds of subpixels. The three subpixels can be of three colors of red (R), green (G), and blue (B) or of three colors of yellow (Y), cyan (C), and magenta (M), for example. Alternatively, the pixel can include four kinds of subpixels. The four subpixels can be of four colors of R, G, B, and white (W) or of four colors of R, G, B, and Y, for example. Specifically, the pixel 703(i,j) can consist of a subpixel 702B(i,j) for blue display, a subpixel 702G(i, j) for green display, and a subpixel 702R(i, j) for red display.

Other than the subpixels including the light-emitting devices, a subpixel including a light-receiving device may also be provided.

FIGS. 11C to 11F illustrate various layout examples of the pixel 703(i, j) including a subpixel 702PS(i, j) including a light-receiving device. The pixel arrangement in FIG. 11C is stripe arrangement, and the pixel arrangement in FIG. 11D is matrix arrangement. The pixel arrangement in FIG. 11E has a structure where three subpixels (the subpixels R, G, and PS) are vertically arranged next to one subpixel (the subpixel B). In the pixel arrangement in FIG. 11F, the vertically oriented three subpixels G, B, and R are arranged laterally, and the subpixel PS and the horizontally oriented subpixel IR are arranged laterally below the three subpixels. Note that the wavelength of light detected by the subpixel 702PS(i, j) is not particularly limited; however, the light-receiving device included in the subpixel 702PS(i, j) preferably has sensitivity to light emitted by the light-emitting device included in the subpixel 702R(i, j), the subpixel 702G(i, j), the subpixel 702B(i, j), or the subpixel 702IR(i, j). For example, the light-receiving device preferably detects one or more kinds of light in blue, violet, bluish violet, green, yellowish green, yellow, orange, red, and infrared wavelength ranges, and the like.

Furthermore, as illustrated in FIG. 11F, a subpixel 702IR(i,j) that emits infrared rays may be added to any of the above-described sets of subpixels in the pixel 703(i, j). Specifically, the subpixel that emits light including light with a wavelength of higher than or equal to 650 nm and lower than or equal to 1000 nm may be used in the pixel 703(i,j).

Note that the arrangement of subpixels is not limited to the structures illustrated in FIGS. 11B to 11F and a variety of arrangement methods can be employed. The arrangement of subpixels may be stripe arrangement, S-stripe arrangement, matrix arrangement, delta arrangement, Bayer arrangement, or PenTile arrangement, for example.

Furthermore, top surfaces of the subpixels may have a triangular shape, a quadrangular shape (including a rectangular shape and a square shape), a polygonal shape such as a pentagonal shape, a polygonal shape with rounded corners, an elliptical shape, or a circular shape, for example. The top surface shape of a subpixel herein refers to a top surface shape of a light-emitting region of a light-emitting device.

Furthermore, in the case where not only a light-emitting device but also a light-receiving device is included in a pixel, the pixel has a light-receiving function and thus can detect a contact or approach of an object while displaying an image. For example, an image can be displayed by using all the subpixels included in a light-emitting apparatus; or light can be emitted by some of the subpixels as a light source and an image can be displayed by using the remaining subpixels.

Note that the light-receiving area of the subpixel 702PS(i, j) is preferably smaller than the light-emitting areas of the other subpixels. A smaller light-receiving area leads to a narrower image-capturing range, prevents a blur in a captured image, and improves the definition. Thus, by using the subpixel 702PS(i, j), high-resolution or high-definition image capturing is possible. For example, image capturing for personal authentication with the use of a fingerprint, a palm print, the iris, the shape of a blood vessel (including the shape of a vein and the shape of an artery), a face, or the like is possible by using the subpixel 702PS(i, j).

Moreover, the subpixel 702PS(i,j) can be used in a touch sensor (also referred to as a direct touch sensor), a near touch sensor (also referred to as a hover sensor, a hover touch sensor, a contactless sensor, or a touchless sensor), or the like. For example, the subpixel 702PS(i, j) preferably detects infrared light. Thus, touch sensing is possible even in a dark place.

Here, the touch sensor or the near touch sensor can detect an approach or contact of an object (e.g., a finger, a hand, or a pen). The touch sensor can detect the object when the light-emitting and light-receiving apparatus and the object come in direct contact with each other. Furthermore, the near touch sensor can detect the object even when the object is not in contact with the light-emitting and light-receiving apparatus. For example, the light-emitting and light-receiving apparatus can preferably detect the object when the distance between the light-emitting and light-receiving apparatus and the object is more than or equal to 0.1 mm and less than or equal to 300 mm, preferably more than or equal to 3 mm and less than or equal to 50 mm. With this structure, light-emitting and light-receiving apparatus can be controlled without the object directly contacting with the light-emitting and light-receiving apparatus. In other words, the light-emitting and light-receiving apparatus can be controlled in a contactless (touchless) manner. With the above-described structure, the light-emitting and light-receiving apparatus can be operated with a reduced risk of being dirty or damaged, or without direct contact between the object and a dirt (e.g., dust, bacteria, or a virus) attached to the light-emitting and light-receiving apparatus.

For high-resolution image capturing, the subpixel 702PS(i, j) is preferably provided in every pixel included in the light-emitting and light-receiving apparatus. Meanwhile, in the case where the subpixel 702PS(i, j) is used in a touch sensor, a near touch sensor, or the like, high accuracy is not required as compared to the case of capturing an image of a fingerprint or the like; accordingly, the subpixel 702PS(i, j) is provided in some subpixels in the light-emitting and light-receiving apparatus. When the number of subpixels 702PS(i, j) included in the light-emitting and light-receiving apparatus is smaller than the number of subpixels 702R(i, j) or the like, higher detection speed can be achieved.

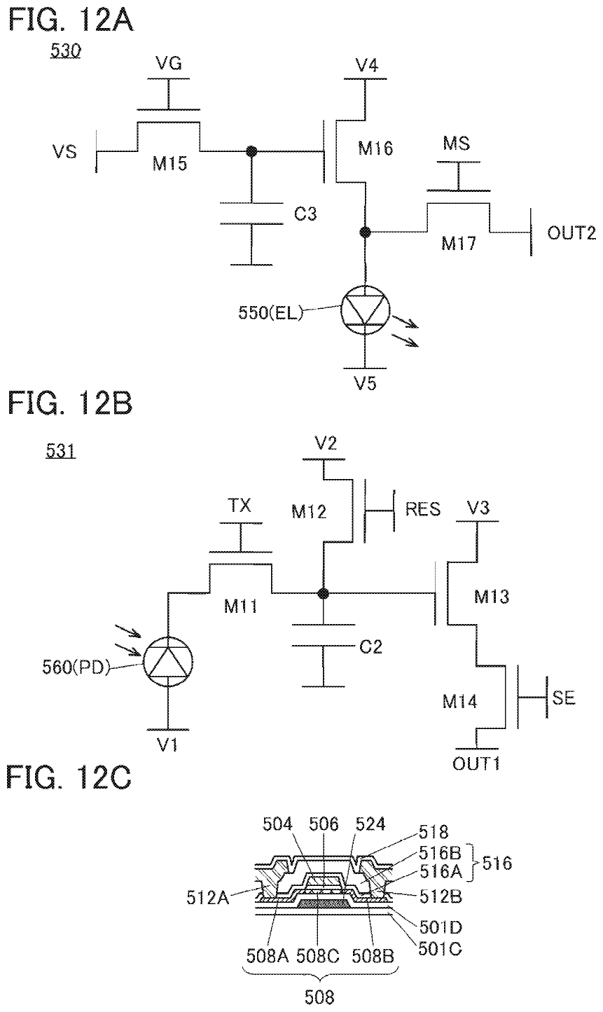

Next, an example of a pixel circuit of a subpixel including the light-emitting device is described with reference to FIG. 12A. A pixel circuit 530 illustrated in FIG. 12A includes a light-emitting device (EL) 550, a transistor M15, a transistor M16, a transistor M17, and a capacitor C3. Note that a light-emitting diode can be used as the light-emitting device 550. In particular, any of the light-emitting devices described in Embodiments 2 and 3 is preferably used as the light-emitting device 550.

In FIG. 12A, a gate of the transistor M15 is electrically connected to a wiring VG, one of a source and a drain of the transistor M15 is electrically connected to a wiring VS, and the other of the source and the drain of the transistor M15 is electrically connected to one electrode of the capacitor C3 and a gate of the transistor M16. One of a source and a drain of the transistor M16 is electrically connected to a wiring V4, and the other is electrically connected to an anode of the light-emitting device 550 and one of a source and a drain of the transistor M17. A gate of the transistor M17 is electrically connected to a wiring MS, and the other of the source and the drain of the transistor M17 is electrically connected to a wiring OUT2. A cathode of the light-emitting device 550 is electrically connected to a wiring V5.

A constant potential is supplied to the wiring V4 and the wiring V5. In the light-emitting device 550, the anode side can have a high potential and the cathode side can have a lower potential than the anode side. The transistor M15 is controlled by a signal supplied to the wiring VG and functions as a selection transistor for controlling a selection state of the pixel circuit 530. The transistor M16 functions as a driving transistor that controls a current flowing through the light-emitting device 550 in accordance with a potential supplied to the gate of the transistor M16. When the transistor M15 is on, a potential supplied to the wiring VS is supplied to the gate of the transistor M16, and the luminance of the light-emitting device 550 can be controlled in accordance with the potential. The transistor M17 is controlled by a signal supplied to the wiring MS and has a function of outputting a potential between the transistor M16 and the light-emitting device 550 to the outside through the wiring OUT2.

Here, a transistor in which a metal oxide (an oxide semiconductor) is used in a semiconductor layer where a channel is formed is preferably used as transistors M15, M16, and M17 included in the pixel circuit 530 in FIG. 12A and transistors M11, M12, M13, and M14 included in a pixel circuit 531 in FIG. 12B.

A transistor using a metal oxide having a wider band gap and a lower carrier density than silicon can achieve an extremely low off-state current. Such a low off-state current enables retention of charges accumulated in a capacitor that is connected in series with the transistor for a long time. Therefore, it is particularly preferable to use a transistor including an oxide semiconductor as the transistors M11, M12, and M15 each of which is connected in series with a capacitor C2 or the capacitor C3. When each of the other transistors also includes an oxide semiconductor, manufacturing cost can be reduced.

Alternatively, transistors using silicon as a semiconductor in which a channel is formed can be used as the transistors M11 to M17. It is particularly preferable to use silicon with high crystallinity such as single crystal silicon or polycrystalline silicon because high field-effect mobility can be achieved and higher-speed operation can be performed.

Alternatively, a transistor including an oxide semiconductor may be used as at least one of the transistors M11 to M17, and transistors including silicon may be used as the other transistors.

Next, an example of a pixel circuit of a subpixel including a light-receiving device is described with reference to FIG. 12B. The pixel circuit 531 illustrated in FIG. 12B includes a light-receiving device (PD) 560, the transistor M11, the transistor M12, the transistor M13, the transistor M14, and the capacitor C2. In the example illustrated here, a photodiode is used as the light-receiving device (PD) 560.

In FIG. 12B, an anode of the light-receiving device (PD) 560 is electrically connected to a wiring V1, and a cathode of the light-receiving device (PD) 560 is electrically connected to one of a source and a drain of the transistor M11. A gate of the transistor M11 is electrically connected to a wiring TX, and the other of the source and the drain of the transistor M11 is electrically connected to one electrode of the capacitor C2, one of a source and a drain of the transistor M12, and a gate of the transistor M13. A gate of the transistor M12 is electrically connected to a wiring RES, and the other of the source and the drain of the transistor M12 is electrically connected to a wiring V2. One of a source and a drain of the transistor M13 is electrically connected to a wiring V3, and the other of the source and the drain of the transistor M13 is electrically connected to one of a source and a drain of the transistor M14. A gate of the transistor M14 is electrically connected to a wiring SE, and the other of the source and the drain of the transistor M14 is electrically connected to a wiring OUT1.

A constant potential is supplied to the wiring V1, the wiring V2, and the wiring V3. When the light-receiving device (PD) 560 is driven with a reverse bias, the wiring V2 is supplied with a potential higher than the potential of the wiring V1. The transistor M12 is controlled by a signal supplied to the wiring RES and has a function of resetting the potential of a node connected to the gate of the transistor M13 to a potential supplied to the wiring V2. The transistor M11 is controlled by a signal supplied to the wiring TX and has a function of controlling the timing at which the potential of the node changes, in accordance with a current flowing through the light-receiving device (PD) 560. The transistor M13 functions as an amplifier transistor for outputting a signal corresponding to the potential of the node. The transistor M14 is controlled by a signal supplied to the wiring SE and functions as a selection transistor for reading an output corresponding to the potential of the node by an external circuit connected to the wiring OUT1.

Although n-channel transistors are illustrated in FIGS. 12A and 12B, p-channel transistors can alternatively be used.

The transistors included in the pixel circuit 530 and the transistors included in the pixel circuit 531 are preferably formed side by side over the same substrate. It is particularly preferable that the transistors included in the pixel circuit 530 and the transistors included in the pixel circuit 531 be periodically arranged in one region

One or more layers including the transistor and/or the capacitor are preferably provided to overlap with the light-receiving device (PD) 560 or the light-emitting device (EL) 550. Thus, the effective area of each pixel circuit can be reduced, and a high-resolution light-receiving portion or display portion can be achieved.

FIG. 12C illustrates an example of a specific structure of a transistor that can be used in the pixel circuit described with reference to FIGS. 12A and 12B. As the transistor, a bottom-gate transistor, a top-gate transistor, or the like can be used as appropriate.

The transistor illustrated in FIG. 12C includes a semiconductor film 508, a conductive film 504, an insulating film 506, a conductive film 512A, and a conductive film 512B. The transistor is formed over an insulating film 501C, for example. The transistor also includes an insulating film 516 (an insulating film 516A and an insulating film 516B) and an insulating film 518.

The semiconductor film 508 includes a region 508A electrically connected to the conductive film 512A and a region 508B electrically connected to the conductive film 512B. The semiconductor film 508 includes a region 508C between the region 508A and the region 508B.

The conductive film 504 includes a region overlapping with the region 508C and has a function of a gate electrode.

The insulating film 506 includes a region positioned between the semiconductor film 508 and the conductive film 504. The insulating film 506 has a function of a first gate insulating film.

The conductive film 512A has one of a function of a source electrode and a function of a drain electrode, and the conductive film 512B has the other.

A conductive film 524 can be used in the transistor. The semiconductor film 508 is positioned between the conductive film 504 and a region included in the conductive film 524. The conductive film 524 has a function of a second gate electrode. An insulating film 501D is positioned between the semiconductor film 508 and the conductive film 524 and has a function of a second gate insulating film.

The insulating film 516 functions as, for example, a protective film covering the semiconductor film 508. Specifically, a film including a silicon oxide film, a silicon oxynitride film, a silicon nitride oxide film, a silicon nitride film, an aluminum oxide film, a hafnium oxide film, an yttrium oxide film, a zirconium oxide film, a gallium oxide film, a tantalum oxide film, a magnesium oxide film, a lanthanum oxide film, a cerium oxide film, or a neodymium oxide film can be used as the insulating film 516, for example.

For the insulating film 518, a material that has a function of inhibiting diffusion of oxygen, hydrogen, water, an alkali metal, an alkaline earth metal, and the like is preferably used. Specifically, the insulating film 518 can be formed using silicon nitride, silicon oxynitride, aluminum nitride, or aluminum oxynitride, for example. In each of silicon oxynitride and aluminum oxynitride, the number of nitrogen atoms contained is preferably larger than the number of oxygen atoms contained.

Note that in a step of forming the semiconductor film used in the transistor of the pixel circuit, the semiconductor film used in the transistor of the driver circuit can be formed. A semiconductor film having the same composition as the semiconductor film used in the transistor of the pixel circuit can be used in the driver circuit, for example.

For the semiconductor film 508, a semiconductor containing a Group 14 element can be used. Specifically, a semiconductor containing silicon can be used for the semiconductor film 508.

Hydrogenated amorphous silicon can be used for the semiconductor film 508. Microcrystalline silicon or the like can also be used for the semiconductor film 508. In such cases, it is possible to provide an apparatus having less display unevenness than an apparatus (including a light-emitting apparatus, a display panel, a display apparatus, and a light-emitting and light-receiving apparatus) using polysilicon for the semiconductor film 508, for example. Moreover, it is easy to increase the size of the apparatus.

Polysilicon can be used for the semiconductor film 508. In this case, for example, the field-effect mobility of the transistor can be higher than that of a transistor using hydrogenated amorphous silicon for the semiconductor film 508. For another example, the driving capability can be higher than that of a transistor using hydrogenated amorphous silicon for the semiconductor film 508. For another example, the aperture ratio of the pixel can be higher than that in the case of employing a transistor using hydrogenated amorphous silicon for the semiconductor film 508.

For another example, the reliability of the transistor can be higher than that of a transistor using hydrogenated amorphous silicon for the semiconductor film 508.

The temperature required for fabricating the transistor can be lower than that required for a transistor using single crystal silicon, for example.

The semiconductor film used in the transistor of the driver circuit can be formed in the same step as the semiconductor film used in the transistor of the pixel circuit. The driver circuit can be formed over a substrate where the pixel circuit is formed. The number of components of an electronic appliance can be reduced.

Single crystal silicon can be used for the semiconductor film 508. In this case, for example, the resolution can be higher than that of a light-emitting apparatus (or a display panel) using hydrogenated amorphous silicon for the semiconductor film 508. For another example, it is possible to provide a light-emitting apparatus having less display unevenness than a light-emitting apparatus using polysilicon for the semiconductor film 508. For another example, smart glasses or ahead-mounted display can be provided.

A metal oxide can be used for the semiconductor film 508. In this case, the pixel circuit can hold an image signal for a longer time than a pixel circuit including a transistor that uses amorphous silicon for the semiconductor film. Specifically, a selection signal can be supplied at a frequency of lower than 30 Hz, preferably lower than 1 Hz, further preferably less than once per minute while flickering is suppressed. Consequently, fatigue of a user of an electronic device can be reduced. Furthermore, power consumption for driving can be reduced.

An oxide semiconductor can be used for the semiconductor film 508. Specifically, an oxide semiconductor containing indium, an oxide semiconductor containing indium, gallium, and zinc, or an oxide semiconductor containing indium, gallium, zinc, and tin can be used for the semiconductor film 508.

The use of an oxide semiconductor for the semiconductor film achieves a transistor having lower leakage current in the off state than a transistor using amorphous silicon for the semiconductor film. Thus, a transistor using an oxide semiconductor for the semiconductor film is preferably used as a switch or the like. Note that a circuit in which a transistor using an oxide semiconductor for the semiconductor film is used as a switch is capable of retaining the potential of a floating node for a longer time than a circuit in which a transistor using amorphous silicon for the semiconductor film is used as a switch.

In the case of using an oxide semiconductor in a semiconductor film, the light-emitting and light-receiving apparatus 720 includes a light-emitting element including an oxide semiconductor in its semiconductor film and having a metal maskless (MML) structure. With this structure, the leakage current that might flow through the transistor and the leakage current that might flow between adjacent light-emitting devices (also referred to as a lateral leakage current, a side leakage current, or the like) can become extremely low. With the structure, a viewer can notice any one or more of the image crispness, the image sharpness, a high chroma, and a high contrast ratio in an image displayed on the display apparatus. When the leakage current that might flow through the transistor and the lateral leakage current that might flow between light-emitting devices are extremely low, display with little leakage of light at the time of black display (so-called black floating) (such display is also referred to as deep black display) can be achieved.

In particular, in the case where a light-emitting device having an MML structure employs the above-described SBS structure, a layer provided between light-emitting devices (for example, also referred to as an organic layer or a common layer which is commonly used between the light-emitting elements) is disconnected; accordingly, display with no or extremely small lateral leakage can be achieved.

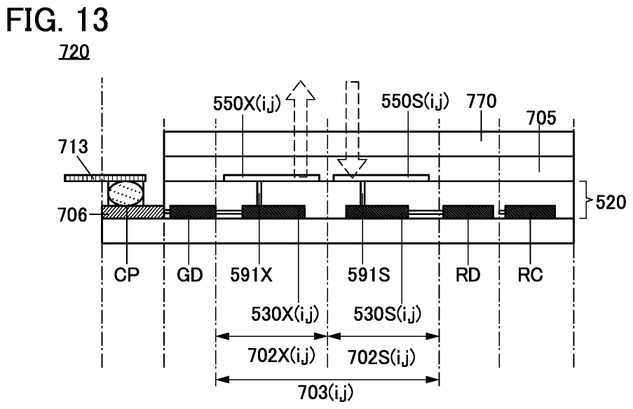

Next, a cross-sectional view of a light-emitting and light-receiving apparatus is shown. FIG. 13 is a cross-sectional view of the light-emitting and light-receiving apparatus illustrated in FIG. 11A.

FIG. 13 is a cross-sectional view of part of the display region 701 including the pixel 703(i,j) and part of a region including the FPC 713 and the wiring 706.

In FIG. 13, the light-emitting and light-receiving apparatus 720 includes the functional layer 520 between the first substrate 510 and the second substrate 770. The functional layer 520 includes, as well as the above-described transistors (M11, M12, M13, M14, M15, M16, and M17), the capacitor (C2 and C3), and the like described with reference to FIGS. 12A to 12C, wirings (VS, VG, V1, V2, V3, V4, and V5) electrically connected to these components, for example. Although the functional layer 520 includes a pixel circuit 530X(i, j), a pixel circuit 530S(i, j), the circuit GD, a circuit RD, a circuit RC, and a conductor CP, in FIG. 13, one embodiment of the present invention is not limited thereto.

Furthermore, each pixel circuit (e.g., the pixel circuit 530X(i, j) and the pixel circuit 530S(i, j) in FIG. 13) included in the functional layer 520 is electrically connected to a light-emitting device and a light-receiving device (e.g., a light-emitting device 550X(i, j) and a light-receiving device 550S(i,j) in FIG. 13) formed over the functional layer 520. Specifically, the light-emitting device 550X(i, j) is electrically connected to the pixel circuit 530X(i, j) through a wiring 591X, and the light-receiving device 550S(i, j) is electrically connected to the pixel circuit 530S(i, j) through a wiring 591S. The insulating layer 705 is provided over the functional layer 520, the light-emitting devices, and the light-receiving device, and has a function of attaching the second substrate 770 and the functional layer 520.

As the second substrate 770, a substrate where touch sensors are arranged in a matrix can be used. For example, a substrate provided with capacitive touch sensors or optical touch sensors can be used as the second substrate 770. Thus, the light-emitting and light-receiving apparatus of one embodiment of the present invention can be used as a touch panel.

The structures described in this embodiment can be used in appropriate combination with any of the structures described in the other embodiments.

### Embodiment 6

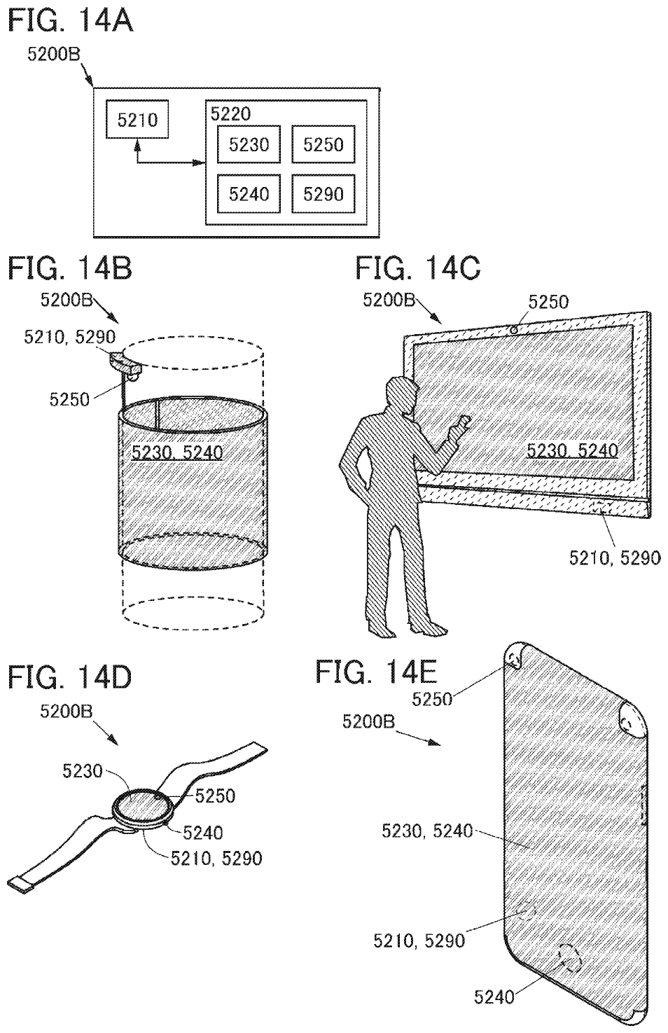

In this embodiment, structures of electronic devices of embodiments of the present invention will be described with reference to FIGS. 14A to 14E, FIGS. 15A to 15E, and FIGS. 16A and 16B. Note that the electronic devices described in this embodiment can each include a light-emitting and light-receiving apparatus of one embodiment of the present invention.

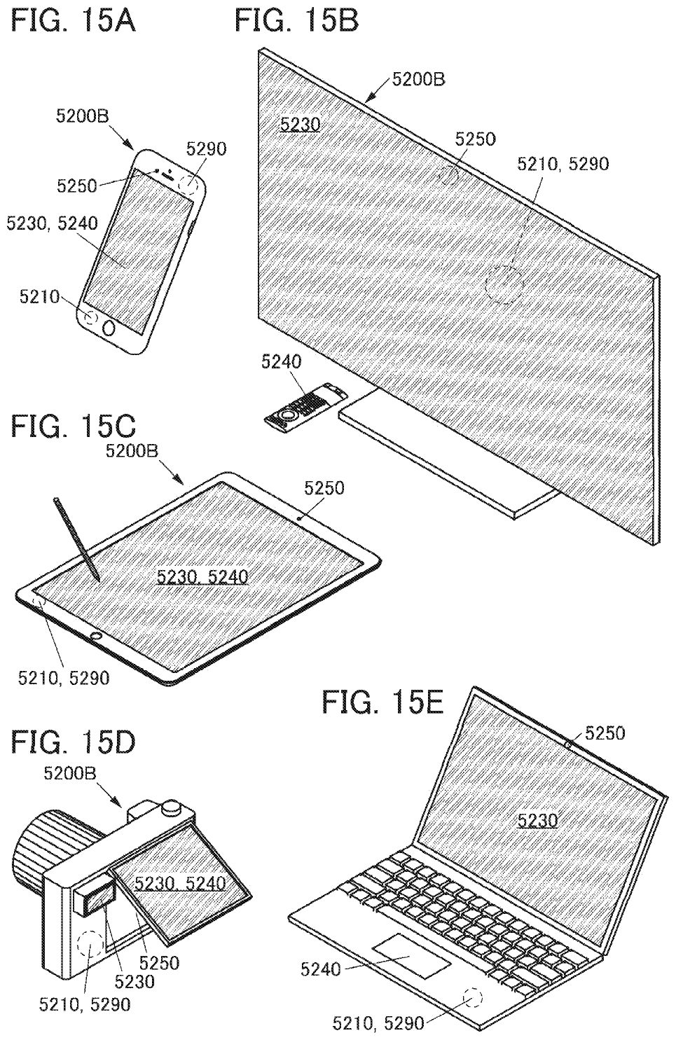

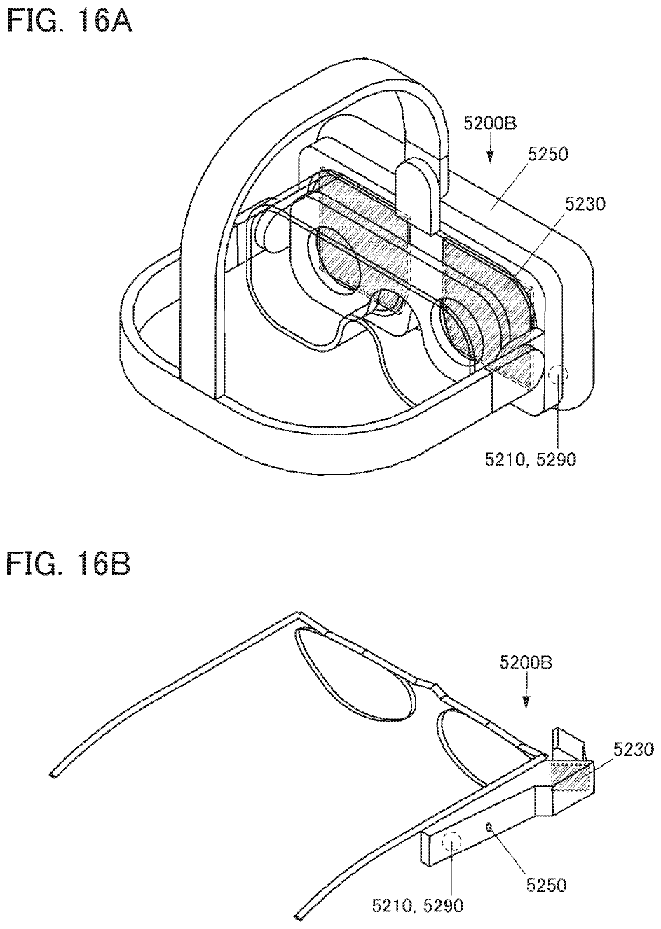

FIGS. 14A to 14E, FIGS. 15A to 15E, and FIGS. 16A and 16B each illustrate a structure of the electronic device of one embodiment of the present invention. FIG. 14A is a block diagram of the electronic device and FIGS. 14B to 14E are perspective views illustrating structures of the electronic device. FIGS. 15A to 15E are perspective views illustrating structures of the electronic device. FIGS. 16A and 16B are perspective views illustrating structures of the electronic device.

An electronic device 5200B described in this embodiment includes an arithmetic device 5210 and an input/output device 5220 (see FIG. 14A).

The arithmetic device 5210 has a function of receiving handling data and a function of supplying image data on the basis of the handling data.

The input/output device 5220 includes a display unit 5230, an input unit 5240, a sensor unit 5250, and a communication unit 5290, and has a function of supplying handling data and a function of receiving image data. The input/output device 5220 also has a function of supplying sensing data, a function of supplying communication data, and a function of receiving communication data.

The input unit 5240 has a function of supplying handling data. For example, the input unit 5240 supplies handling data on the basis of handling by a user of the electronic device 5200B.

Specifically, a keyboard, a hardware button, a pointing device, a touch sensor, an illuminance sensor, an imaging device, an audio input device, an eye-gaze input device, an attitude sensing device, or the like can be used as the input unit 5240.

The display unit 5230 includes a display panel and has a function of displaying image data. For example, the display panel described in Embodiment 4 can be used for the display unit 5230.

The sensor unit 5250 has a function of supplying sensing data. For example, the sensor unit 5250 has a function of sensing a surrounding environment where the electronic device is used and supplying the sensing data.

Specifically, an illuminance sensor, an imaging device, an attitude sensing device, a pressure sensor, a human motion sensor, or the like can be used as the sensor unit 5250.

The communication unit 5290 has a function of receiving and supplying communication data. For example, the communication unit 5290 has a function of being connected to another electronic device or a communication network by wireless communication or wired communication. Specifically, the communication unit 5290 has a function of wireless local area network communication, telephone communication, near field communication, or the like.

FIG. 14B illustrates an electronic device having an outer shape along a cylindrical column or the like. An example of such an electronic device is digital signage. The display panel of one embodiment of the present invention can be used for the display unit 5230. The electronic device may have a function of changing its display method in accordance with the illuminance of a usage environment. The electronic device has a function of changing the displayed content when sensing the existence of a person. Thus, for example, the electronic device can be provided on a column of a building. The electronic device can display advertising, guidance, or the like. The electronic device can be used for digital signage or the like.

FIG. 14C illustrates an electronic device having a function of generating image data on the basis of the path of a pointer used by the user. Examples of such an electronic device include an electronic blackboard, an electronic bulletin board, and digital signage. Specifically, a display panel with a diagonal size of 20 inches or longer, preferably 40 inches or longer, further preferably 55 inches or longer can be used. A plurality of display panels can be arranged and used as one display region. Alternatively, a plurality of display panels can be arranged and used as a multiscreen.

FIG. 14D illustrates an electronic device that is capable of receiving data from another device and displaying the data on the display unit 5230. An example of such an electronic device is a wearable electronic device. Specifically, the electronic device can display several options, and the user can choose some from the options and send a reply to the data transmitter. As another example, the electronic device has a function of changing its display method in accordance with the illuminance of a usage environment. Thus, for example, power consumption of the wearable electronic device can be reduced. As another example, the wearable electronic device can display an image so as to be suitably used even in an environment under strong external light, e.g., outdoors in fine weather.

FIG. 14E illustrates an electronic device including the display unit 5230 having a surface gently curved along a side surface of a housing. An example of such an electronic device is a mobile phone. The display unit 5230 includes a display panel that has a function of displaying images on the front surface, the side surfaces, the top surface, and the rear surface, for example. Thus, a mobile phone can display data on not only its front surface but also its side surfaces, top surface, and rear surface, for example.

FIG. 15A illustrates an electronic device that is capable of receiving data via the Internet and displaying the data on the display unit 5230. An example of such an electronic device is a smartphone. For example, the user can check a created message on the display unit 5230 and send the created message to another device. As another example, the electronic device has a function of changing its display method in accordance with the illuminance of a usage environment. Thus, power consumption of the smartphone can be reduced. As another example, it is possible to obtain a smartphone which can display an image such that the smartphone can be suitably used in an environment under strong external light, e.g., outdoors in fine weather.

FIG. 15B illustrates an electronic device that can use a remote controller as the input unit 5240. An example of such an electronic device is a television system. For example, data received from a broadcast station or via the Internet can be displayed on the display unit 5230. The electronic device can take an image of the user with the sensor unit 5250 and transmit the image of the user. The electronic device can acquire a viewing history of the user and provide it to a cloud service. The electronic device can acquire recommendation data from a cloud service and display the data on the display unit 5230. A program or a moving image can be displayed on the basis of the recommendation data. As another example, the electronic device has a function of changing its display method in accordance with the illuminance of a usage environment. Accordingly, it is possible to obtain a television system which can display an image such that the television system can be suitably used even when irradiated with strong external light that enters the room from the outside in fine weather.

FIG. 15C illustrates an electronic device that is capable of receiving educational materials via the Internet and displaying them on the display unit 5230. An example of such an electronic device is a tablet computer. The user can input an assignment with the input unit 5240 and send it via the Internet. The user can obtain a corrected assignment or the evaluation from a cloud service and have it displayed on the display unit 5230. The user can select suitable educational materials on the basis of the evaluation and have them displayed.

For example, an image signal can be received from another electronic device and displayed on the display unit 5230. When the electronic device is placed on a stand or the like, the display unit 5230 can be used as a sub-display. Thus, for example, it is possible to obtain a tablet computer which can display an image such that the tablet computer is suitably used even in an environment under strong external light, e.g., outdoors in fine weather.

FIG. 15D illustrates an electronic device including a plurality of display units 5230. An example of such an electronic device is a digital camera. For example, the display unit 5230 can display an image that the sensor unit 5250 is capturing. A captured image can be displayed on the sensor unit. A captured image can be decorated using the input unit 5240. A message can be attached to a captured image. A captured image can be transmitted via the Internet. The electronic device has a function of changing shooting conditions in accordance with the illuminance of a usage environment. Accordingly, for example, it is possible to obtain a digital camera that can display a subject such that an image is suitably viewed even in an environment under strong external light, e.g., outdoors in fine weather.

FIG. 15E illustrates an electronic device in which the electronic device of this embodiment is used as a master to control another electronic device used as a slave. An example of such an electronic device is a portable personal computer. For example, part of image data can be displayed on the display unit 5230 and another part of the image data can be displayed on a display unit of another electronic device. Image signals can be supplied. Data written from an input unit of another electronic device can be obtained with the communication unit 5290. Thus, a large display region can be utilized in the case of using a portable personal computer, for example.

FIG. 16A illustrates an electronic device including the sensor unit 5250 that senses an acceleration or a direction. An example of such an electronic device is a goggles-type electronic device. The sensor unit 5250 can supply data on the position of the user or the direction in which the user faces. The electronic device can generate image data for the right eye and image data for the left eye in accordance with the position of the user or the direction in which the user faces. The display unit 5230 includes a display region for the right eye and a display region for the left eye. Thus, a virtual reality image that gives the user a sense of immersion can be displayed on the goggles-type electronic device, for example.

FIG. 16B illustrates an electronic device including an imaging device and the sensor unit 5250 that senses an acceleration or a direction. An example of such an electronic device is a glasses-type electronic device. The sensor unit 5250 can supply data on the position of the user or the direction in which the user faces. The electronic device can generate image data in accordance with the position of the user or the direction in which the user faces. Accordingly, the data can be shown together with a real-world scene, for example. Alternatively, an augmented reality image can be displayed on the glasses-type electronic device.

Note that this embodiment can be combined with any of the other embodiments in this specification as appropriate.

### Example 1

### Synthesis Example 1

In Example 1, a method for synthesizing EtHex-FT2TDMN, which is the organic compound represented by Structural Formula (100) in Embodiment 1, is described. The structural formula of EtHex-FT2TDMN is shown below.

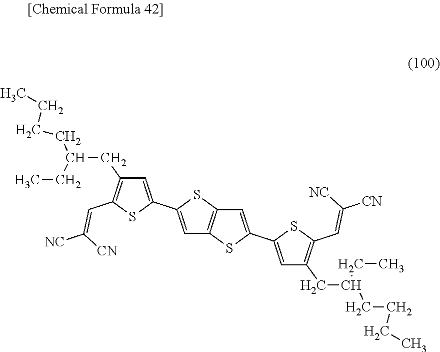 [SMILES: CCCCC(CC)Cc1cc(-c2cc3sc(-c4cc(CC(CC)CCCC)c(C=C(C#N)C#N)s4)cc3s2)sc1C=C(C#N)C#N]

### Step 1: Synthesis of 3-(2-ethylhexyl)-2-thiophenecarbaldehyde

In a 300-mL three-neck flask were put 5.6 g (20 mmol) of 2-bromo-3-(2-ethylhexyl)thiophene and 120 mL of tetrahydrofuran, and the air in the flask was replaced with nitrogen. After this solution was cooled down to −78° C., 14 mL (22 mmol) of a 1.6M hexane solution of n-butyllithium was dropped, which took 10 minutes. Then, stirring was performed at −78° C. for 2 hours. Into this mixture, 1.7 mL of dehydrated N,N-dimethylformamide (dry DMF) was dropped, which took 5 minutes. Then, stirring was performed for 19 hours while bringing the mixture to room temperature.

To the obtained mixture, 100 mL of 1 mol/L hydrochloric acid was added. The obtained mixture was separated into an organic layer and an aqueous layer, and the aqueous layer was subjected to extraction with a mixed solvent of hexane and ethyl acetate (volume ratio of 4:1). The extracted solution and the organic layer were mixed, and the mixed solution was washed twice with water and then washed with a saturated aqueous solution of sodium chloride. The obtained organic layer was dried with magnesium sulfate. The obtained mixture was gravity-filtered to remove the magnesium sulfate. The obtained filtrate was concentrated to give 4.6 g of an orange-colored oily substance. The obtained orange-colored oily substance was purified by silica gel column chromatography (developing solvent with hexane: ethyl acetate=20:1) to give 3.6 g of the target yellowish-orange-colored oily substance in a yield of 80%. Synthesis scheme (a-1) of Step 1 is shown below.

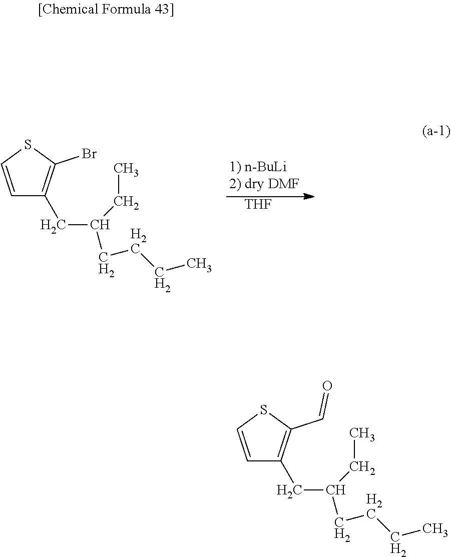

### Step 2: Synthesis of 5-bromo-3-(2-ethylhexyl)-2-thiophenecarbaldehyde

In a 200-mL recovery flask were put 2.5 g (11 mmol) of 3-(2-ethylhexyl)-2-thiophenecarbaldehyde obtained in Step 1, 1.0 g (12 mmol) of sodium hydrogen carbonate, and 45 mL of chloroform, and the mixture was cooled down to 0° C. while being stirred. To this mixture, a solution obtained by mixing 0.57 mL (12 mmol) of bromine and 10 mL of chloroform was dropped, which took 10 minutes. After the dropping, the obtained mixture was stirred for 21 hours while being brought to room temperature.

An aqueous solution of sodium thiosulfate was added to the resulting mixture. The obtained mixture was separated into an organic layer and an aqueous layer, and the organic layer was washed twice with water. Then, the organic layer was washed with sodium hydrogen carbonate, an aqueous solution of sodium thiosulfate, and a saturated aqueous solution of sodium chloride. The obtained organic layer was dried with magnesium sulfate. The obtained mixture was gravity-filtered to remove the magnesium sulfate. The obtained filtrate was concentrated to give 3.2 g of an orange-colored liquid.

It was found from ¹H NMR analysis that the obtained orange-colored liquid was 5-bromo-3-(2-ethylhexyl)-2-thiophenecarbaldehyde, which is the target liquid. Moreover, the NMR spectrum shows that the liquid contained the source material, 3-(2-ethylhexyl)-2-thiophenecarbaldehyde (source material: target=7:100 (molar ratio)). The obtained target liquid was used in the next reaction (Step 3). Synthesis Scheme (a-2) of Step 2 is shown below.

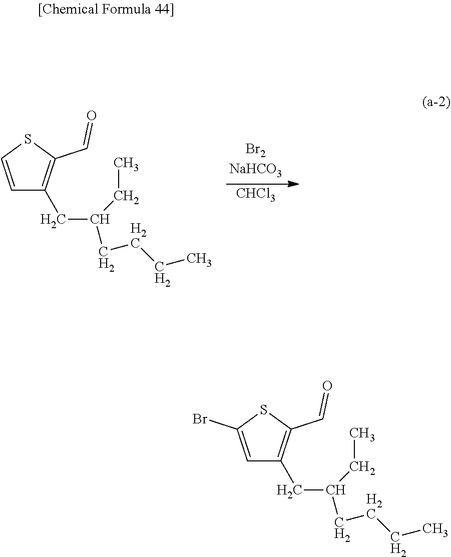 [SMILES: CCCCC(CC)Cc1ccsc1C=O ; "CHCl3" ; CCCCC(CC)Cc1cc(Br)sc1C=O]

### Step 3: Synthesis of 2-[[5-bromo-3-(2-ethylhexyl)-2-thienyl]methylene]propanedinitrile

In a 200-mL recovery flask were put 3.2 g of the orange-colored liquid obtained in Step 2, 0.86 g (13 mmol) of malononitrile, 50 mL of ethanol, and 0.50 mL of triethylamine, and the mixture was heated and refluxed at 80° C. for 2 hours.

The obtained mixture was cooled down to room temperature and then concentrated to give 4.4 g of a mixture including a purple solid and a colorless transparent oily substance. The obtained mixture was purified by silica gel column chromatography (hexane, toluene, and ethyl acetate were used as the developing solvent. The mixing ratio of the solvent was hexane:toluene=1:1 and then changed to hexane:ethyl acetate=1:1.) to give 1.3 g of the target yellowish-orange-colored solid in a yield of 38%. Synthesis scheme (a-3) of Step 3 is shown below.

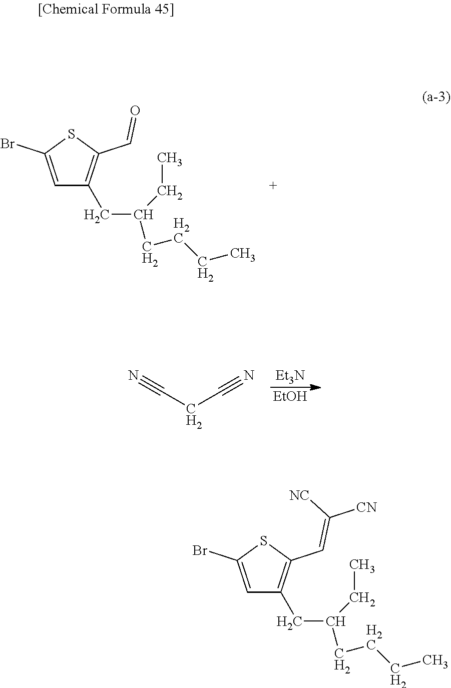 [SMILES: "Et3N" ; "EtOH" ; CCCCC(CC)Cc1cc(Br)sc1C=O ; N#CCC#N ; CCCCC(CC)Cc1cc(Br)sc1C=C(C#N)C#N]

### Step 4: Synthesis of 2,2′-[5,5′-(thieno[3,2-b]thiophene-2,5-diyl)bis(3-(2-ethylhexyl)thiophene-5,2-diyl)]bis(methan-1-yl-1-ylidene)dimalononitrile (EtHex-FT2TDMN)

In a 100-mL three-neck flask were put 0.46 g (1.0 mmol) of 2,5-bis(trimethylstannyl)-thieno[3,2-b]thiophene, 0.98 g (2.7 mmol) of 2-[[5-bromo-3-(2-ethylhexyl)-2-thienyl]methylene]propanedinitrile obtained in Step 3, 48 mg (42 μmol) of tetrakis(triphenylphosphine)palladium(0), and 25 mL of toluene, and the air in the flask was replaced with nitrogen. This mixture was heated and refluxed at 120° C. for 10 hours.

The obtained mixture was cooled down to room temperature, and then the toluene solution was concentrated under reduced pressure. In a 500-mL recovery flask were put the obtained solid, 30 mL of chloroform, and 0.20 L of a saturated aqueous solution of potassium fluoride, and stirring was vigorously performed at room temperature for 16 hours. The obtained mixture was separated into an organic layer and an aqueous layer, and the obtained aqueous layer was subjected to extraction with chloroform. The extracted solution and the organic layer were mixed, and the mixed solution was washed twice with water and then washed with a saturated aqueous solution of sodium chloride. The obtained organic layer was dried with magnesium sulfate. The obtained mixture was gravity-filtered to remove the magnesium sulfate. The obtained filtrate was concentrated to give 1.2 g of a dark purple solid. The obtained solid was washed with acetone and dried to give 0.60 g of a dark purple solid. The obtained mixture was purified by silica gel column chromatography (hexane, ethyl acetate, and chloroform were used as the developing solvent. The mixing ratio of the solvent was ethyl acetate:hexane=1:5 and then changed to ethyl acetate:hexane=1:2, and then chloroform was used.) to give 0.50 g of the target dark purple solid in a yield of 73%. Synthesis scheme (a-4) of Step 4 is shown below.

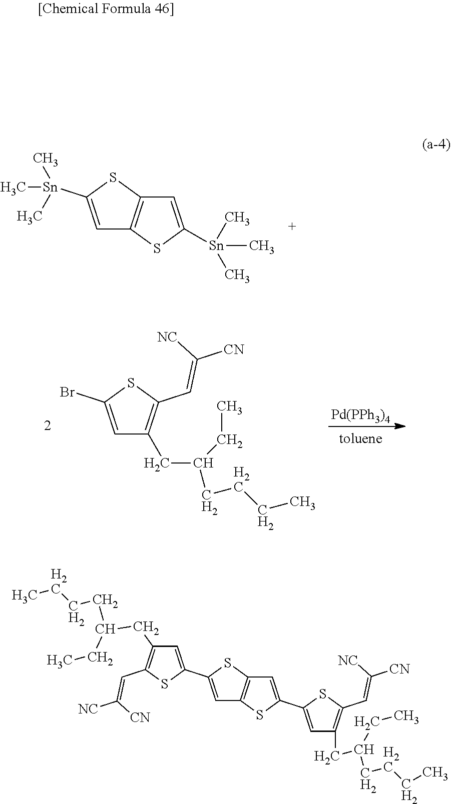

Results of ¹H NMR measurement of the dark purple solid obtained in Step 4 are shown below. The results prove that EtHex-FT2TDMN was obtained in this synthesis example.

¹H NMR (dichloromethane-d₂, 500 MHz): δ =7.87 (s, 2H), 7.64 (s, 2H), 7.21 (s, 2H), 2.69 (d, J=6.9 Hz, 4H), 1.65-1.60 (m, 2H), 1.37-1.28 (m, 16H), 0.92-0.88 (m, 12H).

### Measurement of Physical Properties

Next, ultraviolet-visible absorption spectra (hereinafter simply referred to as “absorption spectra”) and PL emission spectra (hereinafter referred to as “emission spectra”) of EtHex-FT2TDMN in a chloroform solution and a solid thin film of EtHex-FT2TDMN were measured.

The absorption spectrum was measured using an ultraviolet-visible spectrophotometer (V-770DS, manufactured by JASCO Corporation). To calculate the absorption spectrum of EtHex-FT2TDMN in a chloroform solution, the absorption spectrum of chloroform put in a quartz cell was measured and then subtracted from the absorption spectrum of the chloroform solution of EtHex-FT2TDMN put in a quartz cell. The emission spectrum was measured with a fluorescence spectrophotometer (FP-8600DS, manufactured by JASCO Corporation).

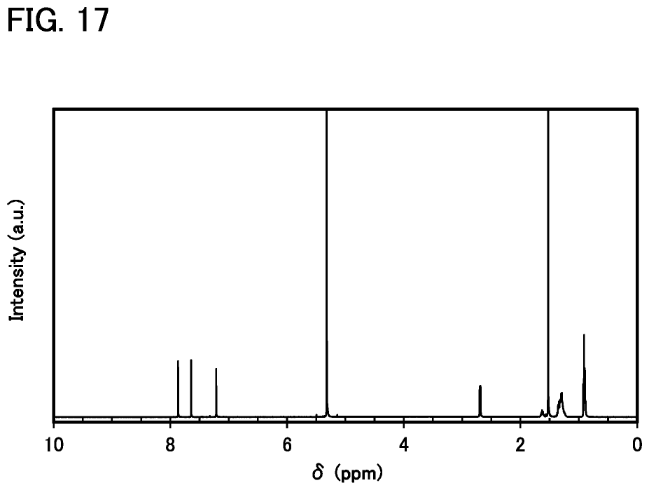

FIG. 17 shows measurement results of the absorption spectrum and the emission spectrum of EtHex-FT2TDMN in the chloroform solution. The horizontal axis represents wavelength, and the vertical axis represents absorption intensity and emission intensity.

As shown in FIG. 17, EtHex-FT2TDMN in the chloroform solution had absorption peaks at around 552 nm and 519 nm and emission wavelength peaks at around 592 nm and 639 nm (excitation wavelength: 519 nm).

The thermogravimetry-differential thermal analysis (TG-DTA) of EtHex-FT2TDMN was performed. The measurement was conducted using a high vacuum differential type differential thermal balance (TG-DTA 2410SA, manufactured by Bruker AXS K.K.). The measurement was performed under two conditions. The first measurement was performed at atmospheric pressure at a temperature rising rate of 10° C./min under a nitrogen stream (flow rate: 200 mL/min). The second measurement was performed at 10 Pa at a temperature rising rate of 10° C./min under a nitrogen stream (flow rate: 2 mL/min).

In the TG-DTA of EtHex-FT2TDMN, the temperature (decomposition temperature) at which the weight obtained by thermogravimetry was reduced by 5% of the weight at the beginning of the measurement was 325° C. at atmospheric pressure and 244° C. at 10 Pa.

Differential scanning calorimetry (DSC) measurement of EtHex-FT2TDMN was performed with DSC8500 manufactured by PerkinElmer, Inc. The DSC measurement was performed in the following manner: the temperature was raised from −10° C. to 300° C. at a temperature rising rate of 40° C./min and held for 3 minutes, and then the temperature was decreased to −10° C. at a temperature decreasing rate of 100° C./min. This operation was performed three times in succession.

The DSC measurement results of the second cycle show that the melting point of EtHex-FT2TDMN is 290° C.

### Example 2

### Synthesis Example 2

In Example 2, a method for synthesizing the organic compound represented by Structural Formula (200) in Embodiment 1 (abbreviation: EtHex-BisDCVTTt) is described. The structural formula of EtHex-BisDCVTTt is shown below.

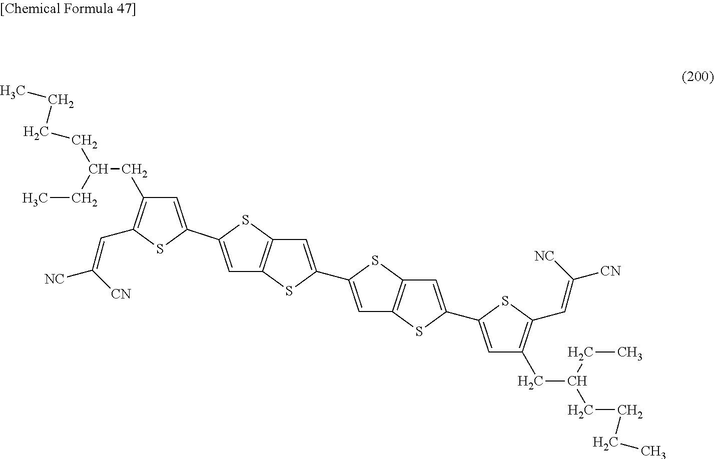 [SMILES: CCCCC(CC)Cc1cc(-c2cc3sc(-c4cc5sc(-c6cc(CC(CC)CCCC)c(C=C(C#N)C#N)s6)cc5s4)cc3s2)sc1C=C(C#N)C#N]

### Step 1: Synthesis of 1,1′-[2,2′-bithieno[3,2-b]thiophene]-5,5′-diylbis[1,1,1-trimethylstannane]

In a 500-mL three-neck flask were put 4.4 g (15 mmol) of 2,2′-bithieno[3,2-b]thiophene and 100 mL of tetrahydrofuran, and the air in the flask was replaced with nitrogen. After this solution was cooled down to −78° C., 22 mL (24 mmol) of a 1.6M hexane solution of n-butyllithium was dropped at the same temperature, which took 10 minutes. After the dropping, the temperature was raised to 0° C. and stirring was performed at the same temperature for 1 hour. Into this mixture kept at 0° C., a solution obtained by dissolving 6.5 g (33 mmol) of trimethyltin chloride in 50 mL of a tetrahydrofuran solution was dropped, which took 15 minutes. After the dropping, the mixture was stirred for 15 hours while being brought to room temperature. The resulting mixture was put into ice water, and the obtained mixture was subjected to extraction with chloroform, so that an organic layer was obtained. The obtained organic layer was washed with a saturated aqueous solution of potassium fluoride and a saturated aqueous solution of sodium chloride in this order. After the washing, the organic layer was dried with magnesium sulfate, and the obtained mixture was suction-filtrated to remove the magnesium sulfate. The obtained filtrate was concentrated to give 9.2 g of a yellow-green solid. This yellow-green solid was recrystallized with 60 mL of toluene and 400 mL of hexane to give 6.0 g of the target yellow-green solid in a yield of 67%. Synthesis scheme (b-1) of Step 1 is shown below.

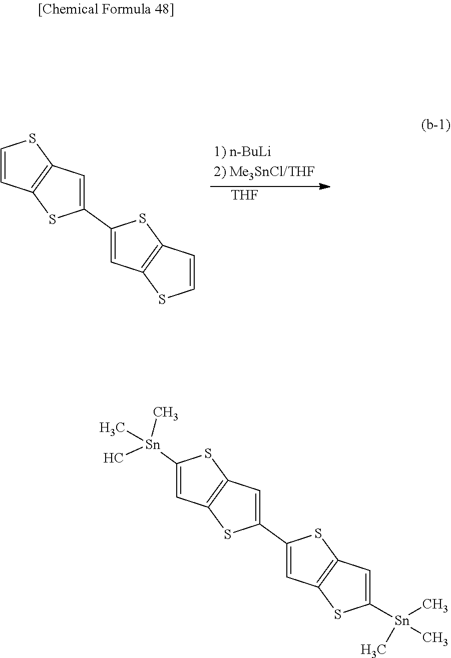

### Step 2: Synthesis of EtHex-BisDCVTTt

In a 50-mL Schlenk flask were put 0.95 g (1.6 mmol) of 1,1′-[2,2′-bithieno[3,2-b]thiophene]-5,5′-diylbis[1,1,1-trimethylstannane] obtained in Step 1, 1.1 g (3.1 mmol) of 2-[[5-bromo-3-(2-ethylhexyl)-2-thienyl]methylene]propanedinitrile synthesized through Step 3 of <<Synthesis example 1>> in Example 1, 0.1 mL of a 10 wt % tri-t-butylphosphine hexane solution, 24 mg (0.11 mmol) of palladium acetate, and 16 mL of toluene, and the air in the flask was replaced with nitrogen. This mixture was heated and refluxed at 120° C. for 7 hours. The obtained mixture was cooled down to room temperature, a saturated aqueous solution of potassium fluoride was added thereto, and stirring was performed for 2 hours. This mixture was suction-filtered to give a black solid. This solid was washed with hot toluene and hot xylene to give 0.44 g of the target black solid in a yield of 34%. Synthesis scheme (b-2) of Step 2 is shown below.

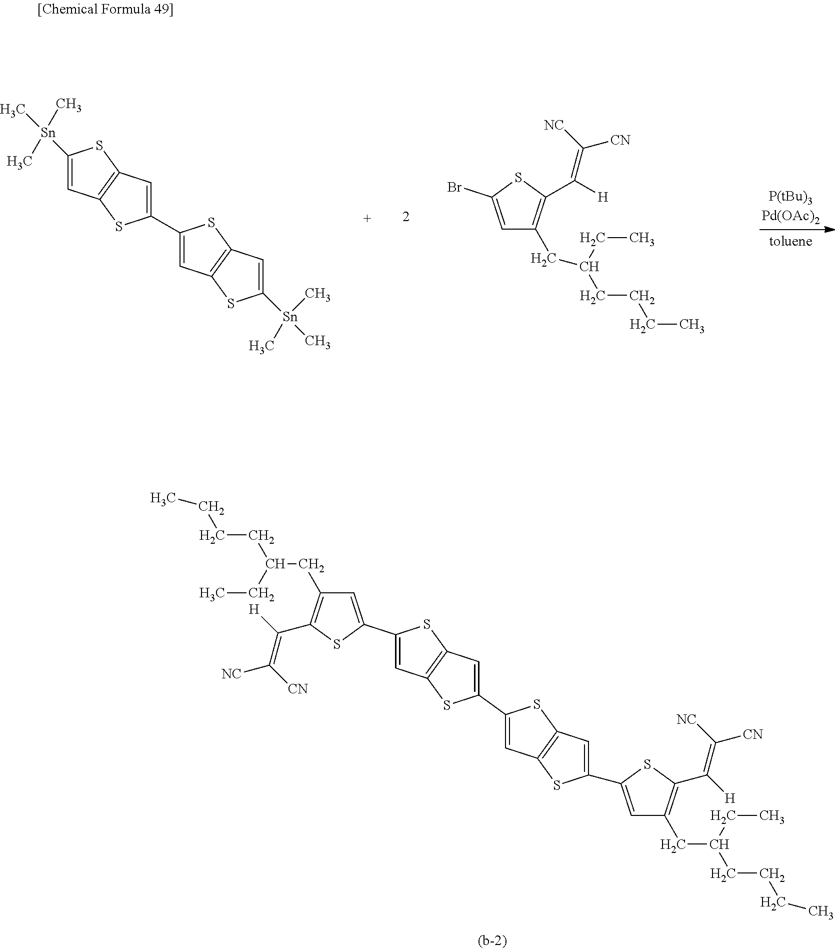

Results of ¹H NMR measurement of the black solid obtained in Step 2 are shown below. The results prove that EtHex-BisDCVTTt was obtained in this synthesis example.

¹H NMR (chloroform-d, 500 MHz): δ =7.80 (s, 2H), 7.60 (s, 2H), 7.47 (s, 2H), 7.12 (s, 2H), 2.67 (d, J=7.5 Hz, 4H), 1.64-1.61 (m, 2H), 1.35-1.23 (m, 16H), 0.94-0.90 (m, 12H).

### Measurement of Physical Properties

Next, ultraviolet-visible absorption spectra (hereinafter simply referred to as “absorption spectra”) and emission spectra of EtHex-BisDCVTTt in a chloroform solution and a solid thin film of EtHex-BisDCVTTt were measured.

Note that the absorption spectrum was measured using an ultraviolet-visible spectrophotometer (V-770DS, manufactured by JASCO Corporation). To calculate the absorption spectrum of EtHex-BisDCVTTt in a chloroform solution, the absorption spectrum of chloroform put in a quartz cell was measured and then subtracted from the absorption spectrum of the chloroform solution of EtHex-BisDCVTTt put in a quartz cell. The emission spectrum was measured with a fluorescence spectrophotometer (FP-8600DS, manufactured by JASCO Corporation).

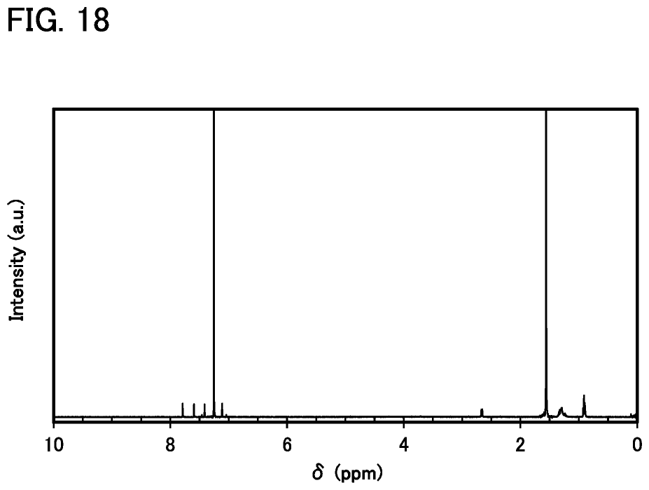

FIG. 18 shows measurement results of the absorption spectrum and the emission spectrum of EtHex-BisDCVTTt in the chloroform solution. The horizontal axis represents wavelength, and the vertical axis represents absorption intensity and emission intensity.

As shown in FIG. 18, EtHex-BisDCVTTt in the chloroform solution had an absorption peak at around 543 nm and emission wavelength peaks at around 638 nm and 689 nm (excitation wavelength: 543 nm).

The thermogravimetry-differential thermal analysis (TG-DTA) of EtHex-BisDCVTTt was performed. The measurement was conducted using a high vacuum differential type differential thermal balance (TG-DTA 2410SA, manufactured by Bruker AXS K.K.). The measurement was performed under two conditions. The first measurement was performed under atmospheric pressure at a temperature rising rate of 10° C./min under a nitrogen stream (flow rate: 200 mL/min). The second measurement was performed at 10 Pa at a temperature rising rate of 10° C./min under a nitrogen stream (flow rate: 2 mL/min).

In the TG-DTA of EtHex-BisDCVTTt, the temperature (decomposition temperature) at which the weight obtained by thermogravimetry was reduced by 5% of the weight at the beginning of the measurement was 362° C. at atmospheric pressure and 295° C. at 10 Pa.

Differential scanning calorimetry (DSC) measurement of EtHex-BisDCVTTt was performed with DSC8500 manufactured by PerkinElmer, Inc. The DSC measurement was performed in the following manner: the temperature was raised from −10° C. to 300° C. at a temperature rising rate of 40° C./min and held for 3 minutes, and then the temperature was decreased to −10° C. at a temperature decreasing rate of 100° C./min. This operation was performed three times in succession.

The DSC measurement results of the first cycle show that the melting point of EtHex-BisDCVTTt is 337° C.

### Example 3

In Example 3, the solubilities of materials that can be used for a photoelectric conversion device of one embodiment of the present invention are described. The solubilities of the materials were measured using a liquid chromatography mass spectrometer.

In Example 3, the solubilities of the organic compound (abbreviation: EtHex-FT2TDMN) represented by Structural Formula (100) in Embodiment 1 and the organic compound (abbreviation: EtHex-BisDCVTTt) represented by Structural Formula (200) in Embodiment 1 were evaluated. As a comparative example, FT2TDMN, which is a material having no branched alkyl, was evaluated. Furthermore, DPAPhA, which is a material with high solubility, was also evaluated as a reference sample.

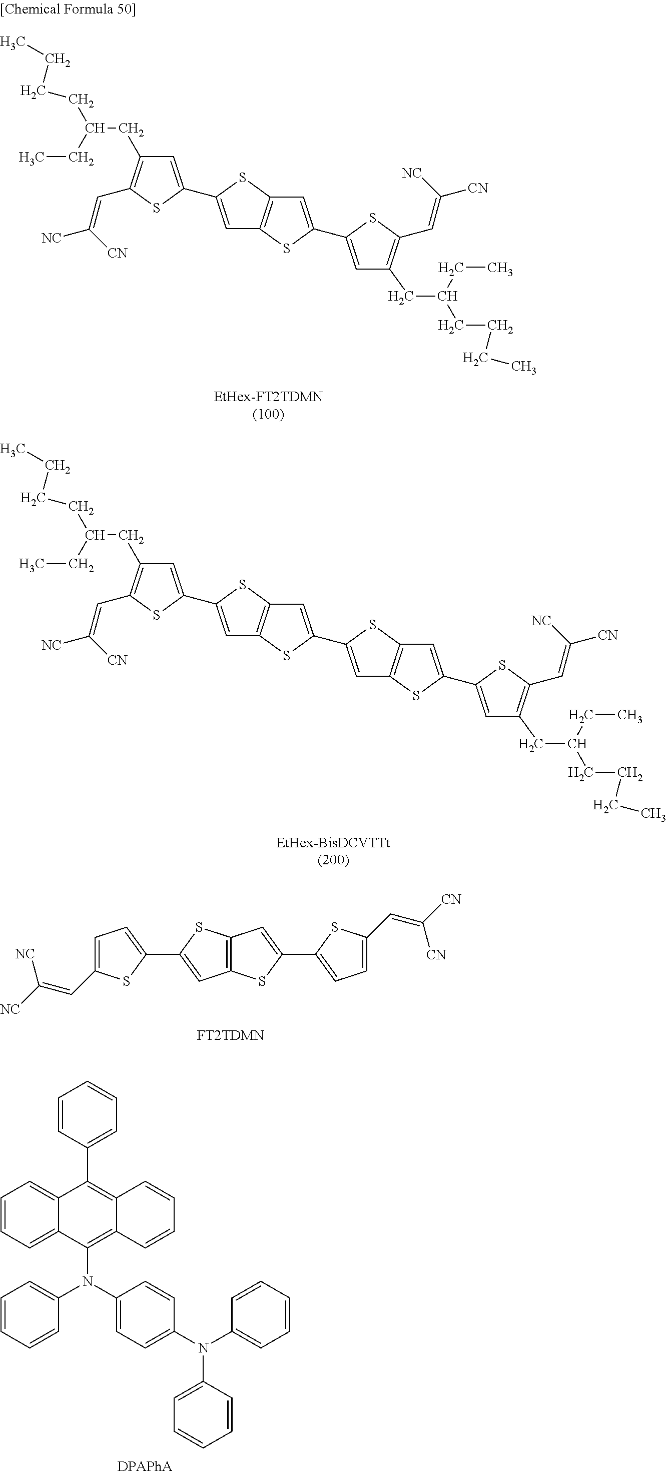

### Solubility Evaluation Method

The solubilities were measured using a liquid chromatography mass spectrometer. Specifically, chromatogram peak areas of the solvent and the material in the sample were obtained, the proportion of the peak area of the material to the peak area of the solvent was calculated, and the calculated value was considered as the solubility.

Note that the solubility of the case where the sample is completely dissolved is 100%. In Example 3, DPAPhA is a reference sample that is completely dissolved. In the case where a chromatogram peak is not detected and an MS spectrum is detected from a sample, the mass of the sample is regarded as being lower than the detection limit and the solubility is 0%.

The liquid chromatography mass spectrometer is constructed of ACQUITY H-Class (produced by Waters Corporation) and Xevo™ G2 Q-TOF MS (produced by Waters Corporation), and ACQUITY UPLC (registered trademark) BEH C8 Column (1.7 m, 2.1×100 mm) (produced by Waters Corporation, hereinafter referred to as Column) was used as a column.

### Formation of Each Sample

Samples of EtHex-FT2TDMN, EtHex-BisDCVTTt, and FT2TDMN used for solubility evaluation were formed. Specifically, in a reagent bottle made of glass were put 0.5 mg of the material and 0.25 mL of chloroform, and ultrasonic treatment was performed for 1 minute. To the obtained mixture, 2.5 mL of acetonitrile was added, and the resulting mixture was left still. After 18 hours, the resulting mixture was filtrated. With the use of the obtained filtrate as a sample, a solubility test for evaluating the solubility was performed using the liquid chromatography mass spectrometer.

Next, the DPAPhA sample used for solubility evaluation was formed. In a reagent bottle made of glass were put 1 mg of the material and 1 mL of toluene, and ultrasonic treatment was performed for 1 minute. Acetonitrile was added to the obtained solution to make a 5-fold dilution. The dilute solution was used as the sample and took the solubility test with the liquid chromatography mass spectrometer.

### Measurement of Solubility of Each Sample

A measurement method for the solubility test for evaluating the solubility using the liquid chromatography mass spectrometer is described. As a mobile phase A and a mobile phase B, acetonitrile and a formic acid aqueous solution (0.1%) were used, respectively; the velocity of flow of a solvent was 0.5 mL/min; and the sample injection amount was 5 μL.

### Measurement Conditions for EtHex-FT2TDMN

For 1 minute from the beginning of the measurement, the ratio of a mobile phase A to a mobile phase B was set to be 75:25. From 1 minute to 9 minutes, measurement was performed with a linear gradient to the ratio of 95:5. After a lapse of 10 minutes, measurement was performed for 5 minutes with the ratio of 95:5 held.

### Measurement Conditions for EtHex-BisDCVTTt

For 1 minute from the beginning of the measurement, the ratio of a mobile phase A to a mobile phase B was set to be 85:15. From 1 minute to 9 minutes, measurement was performed with a linear gradient to the ratio of 95:5. After a lapse of 10 minutes, measurement was performed for 5 minutes with the ratio of 95:5 held.

### Measurement Conditions for FT2TDMN

For 1 minute from the beginning of the measurement, the ratio of a mobile phase A to a mobile phase B was set to be 40:60. From 1 minute to 9 minutes, measurement was performed with a linear gradient to the ratio of 95:5. After a lapse of 10 minutes, measurement was performed for 5 minutes with the ratio of 95:5 held.

### Measurement Conditions for DPAPhA

For 1 minute from the beginning of the measurement, the ratio of a mobile phase A to a mobile phase B was set to be 75:25. From 1 minute to 9 minutes, measurement was performed with a linear gradient to the ratio of 95:5. After a lapse of 10 minutes, measurement was performed for 5 minutes with the ratio of 95:5 held.

### Solubility Measurement Results of Each Sample

The solubilities of the materials are shown in the table below. In the measurement of FT2TDMN, no chromatogram peak was detected and only an MS spectrum was detected.

TABLE 1

| | Ratio of chromatogram peak areas |
|---|---|
| | (Peak area of material/Peak area of |
| Sample name | solvent × 100) [%] |
| FT2TDMN | 0 |
| EtHex-FT2TDMN | 18 |
| EtHex-BisDCVTTt | 3.5 |
| DPAPhA | 100 |

Compared with FT2TDMN, EtHex-FT2TDMN and EtHex-BisDCVTTt showed favorable solubilities. In particular, it was found that the solubility of EtHex-FT2TDMN, where branched alkyl groups are bonded to FT2TDMN, was improved from that of FT2TDMN. This confirms that substitution of branched alkyl groups has an effect of improving the solubility of the FT2TDMN derivative.

This application is based on Japanese Patent Application Serial No. 2022-037934 filed with Japan Patent Office on Mar. 11, 2022, the entire contents of which are hereby incorporated by reference.

What is claimed is:

1. An organic compound represented by General Formula (G1): 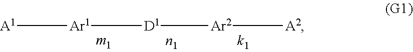
wherein D¹ represents a substituted or unsubstituted thiophene-diyl group, a substituted or unsubstituted furan-diyl group, a substituted or unsubstituted thiophene-containing heteroarylene group having 4 to 30 carbon atoms, or a substituted or unsubstituted furan-containing heteroarylene group having 4 to 30 carbon atoms,
wherein Ar¹ and Ar² each independently represent a heteroarylene group having 2 to 30 carbon atoms and having 1 or more substituents or an arylene group having 6 to 30 carbon atoms and having 1 or more substituents,
wherein A¹ and A² each independently represent hydrogen, deuterium, a nitro group, a substituted or unsubstituted alkyl group having 1 to 6 carbon atoms, a halogen, a substituted or unsubstituted alkyl halide group having 1 to 6 carbon atoms, a cyano group, a substituted or unsubstituted alkoxy group having 1 to 6 carbon atoms, a vinyl group having 1 to 3 substituents, or a formyl group,
wherein n₁ represents an integer of 1 or more,
wherein m₁ and k₁ each independently represent an integer of 0 to 3,
wherein at least one of the substituents that D¹, Ar¹, and Ar² have has a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms, and
wherein in the case where m₁ and k₁ are 0, at least one of the substituents that D¹ has a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms.

2. The organic compound according to claim 1,
wherein D¹ is represented by any one of General Formulas (g1-1-1) to (g1-1-5): 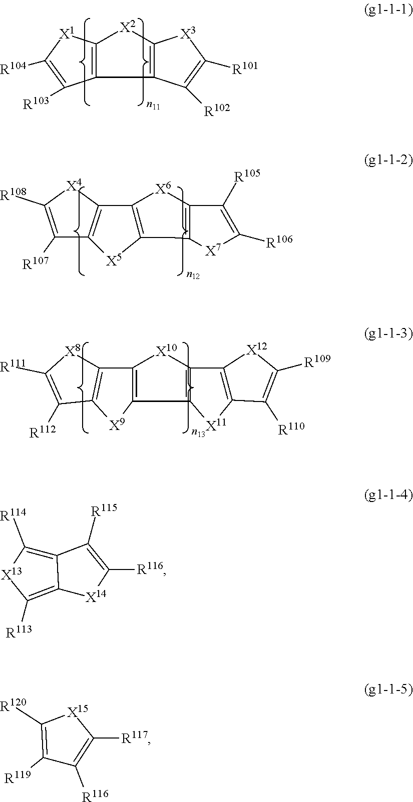
wherein n₁₁ represents an integer of 0 to 10,
wherein n₁₂ and n₁₅ each independently represent an integer of 0 to 4,
wherein X¹ to X¹⁵ each independently represent oxygen or sulfur,
wherein one of R¹⁰¹ and R¹⁰², one of R¹⁰⁵ and R¹⁰⁶, or one of R¹⁰⁹ and R¹¹⁰ is bonded to one of Ar¹ or A¹ and Ar² or A²,
wherein one of R¹⁰³ and R¹⁰⁴, one of R¹⁰⁷ and R¹⁰⁸, or one of R¹¹¹ and R¹¹² is bonded to the other of Ar¹ or A¹ and Ar² or A²,
wherein any one of R¹¹³ to R¹¹⁶ is bonded to Ar¹ or A¹,
wherein another one of R¹¹³ to R¹¹⁶ is bonded to Ar² or A²,
wherein any one of R¹¹⁷ to R¹²⁰ is bonded to Ar¹ or A¹,
wherein another one of R¹¹⁷ to R¹²⁰ is bonded to Ar² or A², and
wherein groups bonded to none of Ar¹, A¹, Ar², and A² among R¹⁰¹ to R¹²⁰ each independently represent hydrogen, deuterium, a branched alkyl group having 3 to 20 carbon atoms, a straight-chain alkyl group having 7 or more carbon atoms, a cycloalkyl group having 3 to 10 carbon atoms, a branched alkoxy group having 3 to 20 carbon atoms, a straight-chain alkoxy group having 7 or more carbon atoms, a substituted or unsubstituted aryl group having 6 to 30 carbon atoms, a substituted or unsubstituted heteroaryl group having 2 to 30 carbon atoms, a branched alkyl halide group having 3 to 20 carbon atoms, a straight-chain alkyl halide group having 7 or more carbon atoms, or a halogen.

3. The organic compound according to claim 1,
wherein each of A¹ and A² is represented by General Formula (g1-2): 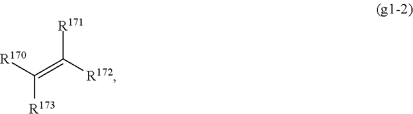
wherein R¹⁷⁰ to R¹⁷² each independently represent hydrogen, deuterium, a cyano group, fluorine, chlorine, a nitro group, a substituted or unsubstituted alkyl halide group having 1 to 6 carbon atoms, or a substituted or unsubstituted alkoxy group having 1 to 6 carbon atoms, and
wherein R¹⁷³ is bonded to one of Ar¹ and Ar² or D¹.

4. The organic compound according to claim 1,
wherein Ar¹ and Ar² each independently represent a substituted or unsubstituted thiophene-diyl group, a substituted or unsubstituted furan-diyl group, a substituted or unsubstituted phenylene group, or a substituted or unsubstituted naphthalene-diyl group.

5. An organic compound represented by any one of General Formulas (G1-1) to (G1-3): 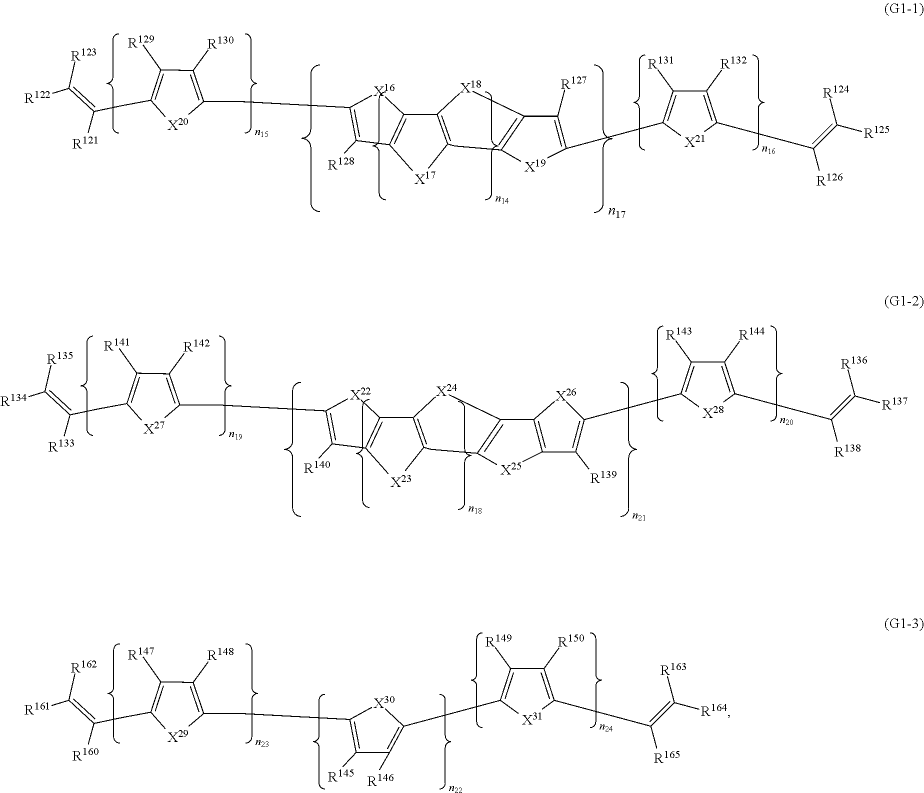
wherein X¹⁶ to X³¹ each independently represent oxygen or sulfur,
wherein n₁₄, n₁₈, and n₂₂ each independently represent an integer of 0 to 4,
wherein n₁₅, n₁₆, n₁₉, n₂₀, n₂₃, and n₂₄ each independently represent an integer of 0 to 3,
wherein n₁₇, n₂₁, and n₂₂ represent an integer of 1 to 3,
wherein R¹²⁷ to R¹³², R¹³⁹ to R¹⁴⁴, and R¹⁴⁵ to R¹⁵⁰ each independently represent hydrogen, deuterium, a branched alkyl group having 3 to 20 carbon atoms, a straight-chain alkyl group having 7 or more carbon atoms, a cycloalkyl group having 3 to 10 carbon atoms, a branched alkoxy group having 3 to 20 carbon atoms, a straight-chain alkoxy group having 7 or more carbon atoms, a substituted or unsubstituted aryl group having 6 to 30 carbon atoms, a substituted or unsubstituted heteroaryl group having 2 to 30 carbon atoms, a branched alkyl halide group having 3 to 20 carbon atoms, a straight-chain alkyl halide group having 7 or more carbon atoms, or a halogen,
wherein at least one of R^{12′} to R¹³², at least one of R¹³⁹ to R¹⁴⁴, and at least one of R¹⁴⁵ to R¹⁵⁰ each independently represent a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms,
wherein R¹²¹ to R¹²⁶, R¹³³ to R¹³⁸, and R¹⁶⁰ to R¹⁶⁵ each independently represent hydrogen, deuterium, a cyano group, fluorine, chlorine, a substituted or unsubstituted alkyl halide group having 1 to 6 carbon atoms, or a substituted or unsubstituted alkoxy group having 1 to 6 carbon atoms, and
wherein at least one of R¹²¹ to R¹²⁶, at least one of R¹³³ to R¹³⁸, and at least one of R¹⁶⁰ to R¹⁶⁵ represent a cyano group, fluorine, chlorine, a nitro group, a substituted or unsubstituted alkyl halide group having 1 to 6 carbon atoms, or a substituted or unsubstituted alkoxy group having 1 to 6 carbon atoms.

6. An organic compound represented by Structural Formula (100) or Structural Formula (200):

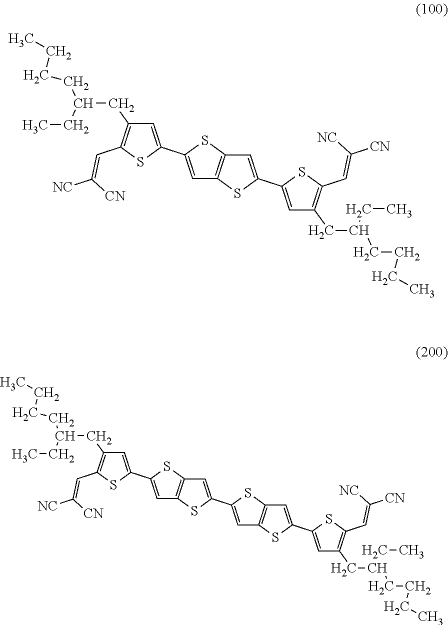

7. A light-receiving device comprising:
a first electrode;
a second electrode; and
an organic compound layer,
wherein the organic compound layer is positioned between the first electrode and the second electrode,
wherein the organic compound layer comprises an organic compound,
wherein the organic compound is represented by General Formula (G1): 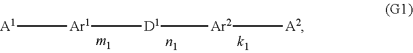
wherein D¹ represents a substituted or unsubstituted thiophene-diyl group, a substituted or unsubstituted furan-diyl group, a substituted or unsubstituted thiophene-containing heteroarylene group having 4 to 30 carbon atoms, or a substituted or unsubstituted furan-containing heteroarylene group having 4 to 30 carbon atoms,
wherein Ar¹ and Ar² each independently represent a heteroarylene group having 2 to 30 carbon atoms and having 1 or more substituents or an arylene group having 6 to 30 carbon atoms and having 1 or more substituents,
wherein A¹ and A² each independently represent hydrogen, deuterium, a nitro group, a substituted or unsubstituted alkyl group having 1 to 6 carbon atoms, a halogen, a substituted or unsubstituted alkyl halide group having 1 to 6 carbon atoms, a cyano group, a substituted or unsubstituted alkoxy group having 1 to 6 carbon atoms, or a vinyl group having 1 to 3 substituents,
wherein n₁ represents an integer of 1 or more,
wherein m₁ and k₁ each independently represent an integer of 0 to 3,
wherein at least one of the substituents that D¹, Ar¹, and Ar² have has a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms, and
wherein in the case where m₁ and k₁ are 0, at least one of the substituents that D¹ has a branched alkyl group having 3 to 20 carbon atoms, a straight-chain alkyl group having 7 or more carbon atoms, a branched alkoxy group having 3 to 20 carbon atoms, a straight-chain alkoxy group having 7 or more carbon atoms, a branched alkyl halide group having 3 to 20 carbon atoms, or a straight-chain alkyl halide group having 7 or more carbon atoms.

8. The light-receiving device according to claim 7,
wherein the organic compound layer is an active layer.

9. The light-receiving device according to claim 7,
wherein the organic compound layer is an electron-transport layer.

10. The light-receiving device according to claim 7, further comprising a light-emitting layer.

11. A light-emitting and light-receiving apparatus comprising:
the light-receiving device according to claim 8; and
a light-emitting device.

12. The light-emitting and light-receiving apparatus according to claim 11, further comprising at least one of a transistor and a substrate.

13. An electronic device comprising:
the light-emitting and light-receiving apparatus according to claim 12; and
at least one of a microphone, a camera, an operation button, a connection terminal, and a speaker.

14. The organic compound according to claim 2,
wherein each of A¹ and A² is represented by General Formula (g1-2): 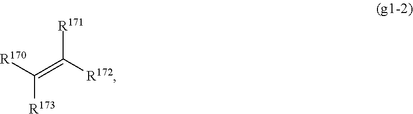
wherein R¹⁷⁰ to R¹⁷² each independently represent hydrogen, deuterium, a cyano group, fluorine, chlorine, a nitro group, a substituted or unsubstituted alkyl halide group having 1 to 6 carbon atoms, or a substituted or unsubstituted alkoxy group having 1 to 6 carbon atoms, and
wherein R¹⁷³ is bonded to one of Ar¹ and Ar² or D¹.

15. The organic compound according to claim 2,
wherein Ar¹ and Ar² each independently represent a substituted or unsubstituted thiophene-diyl group, a substituted or unsubstituted furan-diyl group, a substituted or unsubstituted phenylene group, or a substituted or unsubstituted naphthalene-diyl group.

16. A light-emitting and light-receiving apparatus comprising:
the light-receiving device according to claim 9; and
a light-emitting device.

17. A light-emitting and light-receiving apparatus comprising:
the light-receiving device according to claim 10; and
a light-emitting device.
